(19) United States
(12) Patent Application Publication
(10) Pub. No.: US 2023/0346911 A1
(43) Pub. Date: Nov. 2, 2023

(54) BROADLY PROTECTIVE INFLUENZA B VIRUS VACCINES

(71) Applicant: Wisconsin Alumni Research Foundation, Madison, WI (US)

(72) Inventors: Yoshihiro Kawaoka, Middleton, WI (US); Gabriele Neumann, Madison, WI (US)

(21) Appl. No.: 18/173,535

(22) Filed: Feb. 23, 2023

Related U.S. Application Data
(60) Provisional application No. 63/313,073, filed on Feb. 23, 2022.

(51) Int. Cl. A61K 39/145; A61P 37/04; C12N 15/10

(52) U.S. Cl. CPC A61K 39/145; A61P 37/04; C12N 15/1082

(57) ABSTRACT

Methods of generating influenza B viruses that include a plurality of residues in HA that are specific for the Yamagata or Victoria lineage of influenza B viruses, and recombinant viruses having a plurality of residues in HA that are specific for the Yamagata lineage and for the Victoria lineage of influenza B viruses.

### CROSS-REFERENCE TO RELATED APPLICATIONS

This application claims the benefit of the filing date of U.S. application No. 63/313,073, filed on Feb. 23, 2022, the disclosure of which is incorporated by reference herein.

### STATEMENT REGARDING FEDERALLY SPONSORED RESEARCH OR DEVELOPMENT

This invention was made with government support under AI159937 awarded by the National Institutes of Health and under HHSO100201500033C awarded by the Biomedical Advanced Research and Development Authority (BARDA). The government has certain rights in the invention.

### INCORPORATION BY REFERENCE OF SEQUENCE LISTING

A Sequence Listing is provided herewith as an xml file, “2332497.xml” created on May 8, 2023, and having a size of 32,817 bytes. The content of the xml file is incorporated by reference herein in its entirety.

### BACKGROUND

Influenza viruses (belonging to the family Orthomyxoviridae) frequently cause respiratory infections in humans. Four genera (types A-D) are currently recognized, but only influenza A viruses (IAV) and influenza B viruses (IBV) are of medical relevance to humans. Based on the antigenic properties of the two viral surface glycoproteins, hemagglutinin (HA) and neuraminidase (NA), IAVs are divided into 18 HA (H1-18) and 11 NA (N1-N11) subtypes. Viruses of subtypes H1N1 and H3N2 currently circulate among humans causing annual epidemics. IBVs are not divided into subtypes, but two genetically and antigenically distinct lineages circulate in humans and cause frequent epidemics. The Centers for Disease Control and Prevention estimate that influenza virus infections account for 9-45 million illnesses, 140,000-810,000 hospitalizations, and 12,000-61,000 deaths annually in the US alone (https://www.cdc.gov/flu/about/burden/index.html), resulting in substantial economic costs.

Influenza B virus (IBV) was first isolated in 1940, seven years after the identification of IAV in 1933, and found to cause epidemics every 2-4 years. IBVs separated into two genetically and antigenically distinct lineages in the early 1980's; these lineages are named ‘Victoria’ and ‘Yamagata’ after the B/Victoria/2/87 and B/Yamagata/16/88 strains, respectively. Until the early 2000's, viruses of the Victoria lineage were primarily restricted to Asia, whereas Yamagata-lineage viruses circulated worldwide. In the early 2000's, viruses of the Victoria lineage started to spread worldwide, and IBVs of both lineages have been co-circulating globally. Despite this global co-circulation, there are local and temporal differences in the prevalence of the two lineages.

IAV and IBV infections are clinically indistinguishable and there is now ample evidence that IBV causes substantial morbidity and mortality. As an added factor, IBV epidemics tend to occur late in the winter season, when immune responses to vaccines may start to wane. Overall, the IBV disease burden is estimated to be lower than that of H3N2 IAV, but higher than that of H1N1 IAV. Children under the age of 10 have a higher IBV disease burden than any other age group as underscored by the finding that 22%-44% of influenza deaths in children in the US from 2004-2011 (excluding the 2009-2010 pandemic season) were caused by IBVs.

Human influenza vaccines present the HA of the selected vaccine strain as an inactivated vaccine (the most commonly used influenza vaccines), live-attenuated vaccine, or recombinant HA protein. Traditionally, influenza vaccines have included three components, representing H1N1 and H3N2 IAVs, and an IBV. However, vaccines directed against one of the IBV lineages induce only low levels of cross-reactive antibodies against the other IBV lineage. Between the 2001-2002 and 2010-2011 seasons, the IBV lineage selected for the human trivalent vaccine matched the dominant circulating lineage in only five out of ten seasons, resulting in low vaccine efficacy. Therefore, since 2012, the WHO has recommended the inclusion of both IBV lineages in quadrivalent vaccine formulations.

The efficacy of influenza vaccines typically does not exceed about 60/6-70% and, in fact, is considerably lower in most years. One reason for this is an antigenic mismatch between the selected vaccine virus and the strain circulating at the peak of the influenza season. The high mutation rate of IAVs causes frequent mutations in the five major antigenic epitopes of IAV HAs (all located in the head region of HA), resulting in antigenic escape variants that are not neutralized by the antibody repertoire in human populations. If antigenic escape variants emerge after the selection of the vaccine strains, the vaccine efficacy will be low.

In contrast to IAVs, relatively little is known about the antigen epitopes and antigenic evolution of IBVs. IBVs have a lower evolutionary rate than IAVs resulting in less antigenic drift and less frequent updates of the vaccine strains. No comprehensive studies have been done to map the antigenic epitopes of IBV, but crystallographic structures of IBV HAs in complex with broadly reactive human mAbs revealed mAb binding sites in an area overlapping the receptor-binding site, in the vestigial esterase domain (located underneath the HA head but above the so-called HA stem region), and in the highly conserved HA stem.

The influenza B viruses circulating in humans fall into two genetically and antigenically distinct lineages, i.e., the Victoria- and Yamagata-lineages. Vaccines that protect against viruses from one lineage do not provide efficient protection against viruses from the other lineages. As a consequence, many influenza vaccines are now quadrivalent, i.e., they are composed of four different vaccine strains representing influenza A/H3N2 and A/H1N1 viruses, and both influenza B virus lineages. Efforts are therefore underway to develop broadly protective vaccines that protect against influenza B viruses of both lineages.

### SUMMARY

As described herein, a series of mutant influenza B viruses was developed that incorporated mutations in the HA1 protein that cause the virus to react with sera generated via exposure to the opposite lineage of influenza B viruses, in addition to reacting to sera generated via exposure to the parental lineage of influenza B viruses. As the Yamagata and Victoria lineages are antigenically distinct, these mutant viruses are antigenically ‘in between’ the two lineages.

For example, 37 amino acid differences were identified between the antigenically distinct HA1 proteins from one example of each of the Victoria and Yamagata lineages, and a library was designed in which the amino acid residues that occupy each of those locations were mutated to represent all combinations of the parental amino acids at that position which included the two choices found at that residue, e.g., one from each lineage, and rescued viruses were those with a functional HA. One library was generated in a Yamagata lineage background and another library was generated in a Victoria lineage background. In this way, mutants with any number of substitutions (up to 37) were created, and each large library was subjected to plaque assays. Individual viral plaques were picked, followed by sequence analysis of the HA genes. Mutant viruses were then screened for cross-reactivity with both Yamagata-specific and Victoria-specific sera. A few dozen mutants were identified with substantially higher cross-reactivity than seen with the parent virus. In one embodiment, the mutants have from 2 to 40, 5 to 15, 12 to 25, or 15 to 30 residues at those positions that are representative of a Yamagata lineage HA or a Victoria lineage HA. For example, in a Yamagata lineage, at the recited positions there may be up to 3, 4, 5, 6, 7, 9, 10, 11, 12, 13, 14, 15, 20, 25 or more residues that are representative of residues from the Victoria lineage found at the corresponding positions, and in a Victoria lineage, at the recited positions there may be up to 3, 4, 5, 6, 7, 9, 10, 11, 12, 13, 14, 15, 20, 25 or more residues that are representative of residues from the Yamagata lineage found at the corresponding positions. Some of he IBV HA mutants that have acquired reactivity with serum raised against a virus of the other lineage, while maintaining reactivity with serum raised against the homologous virus, were then used to induce an immune reaction in ferrets to generate anti-sera for each of the mutants, which sera tested for reactivity against circulating Victoria- and Yamagata-viruses, to determine whether the mutant ‘in between’ viruses induce antibodies that react with viruses from both lineages. The use of such mutants as vaccine viruses may thus elicit antibodies that provide protection against influenza B viruses from both lineages, and thereby obviate the need for flu vaccines to include two separate B viruses.

In one embodiment, the disclosure provides for methods of making ‘in between’ influenza B viruses, a composition with at least one of the ‘in between’ viruses, e.g., useful as a vaccine, such as in a multivalent influenza vaccine, and methods of immunizing mammals such as humans with the composition, for example, a trivalent cocktail of two influenza A viruses and an ‘in between’ influenza B virus. The HA containing or expressing composition may include non-native display vehicles for the ‘in between’ influenza B HAs, e.g., a recombinant virus such as a recombinant influenza virus, which may be live or an inactivated virus, nanoparticles, such as inorganic nanoparticles or organic nanoparticles, which may be formed from polymers and optionally are 100 nm or less in diameter, including for example liposomes, including lipid or protein based nanoparticles, self-assembling protein nanoparticles, mRNA encoding the ‘in between’ HA, or virus-like particles (VLPs), or may include isolated HA protein from an ‘in between’ virus. For example, nanoparticles may present the antigen at high concentrations, may protect the native structure of the antigen, and may improve antigen delivery to antigen-presenting cells. Nanoparticles may also stimulate cellular immune response pathways. The ‘in between’ influenza B viruses may have, for example, a four-fold or greater, increase in HI titers to a serum raised against a virus from the other lineage (relative to the parental virus), and no more than a four-fold decrease in HI titers to the homologous serum. In one embodiment, the ‘in between’ influenza B virus has an antibody titer of 80 or more against viruses from both lineages.

The disclosure provides a method to prepare a broadly reactive influenza B virus hemagglutinin (HA), comprising: introducing mutations at a plurality of nucleotides in an isolated parental influenza virus nucleic acid molecule encoding an influenza B virus hemagglutinin (HA), thereby providing a library of influenza virus nucleic acid molecules encoding a mutant influenza B virus hemagglutinin, wherein the plurality of mutations in the influenza B virus hemagglutinin results in a plurality of amino acid substitutions or a deletion of a codon, introducing the library into cells so as to provide a library of cells that express the mutant hemagglutinins; and identifying a mutant hemagglutinin encoded by the library that is recognized by anti-Yamagata lineage specific sera and anti-Victoria lineage specific sera as a result of one or more substitutions and/or deletions at the one or more of the positions.

In one embodiment, the disclosure provides a method to prepare a broadly reactive influenza B virus hemagglutinin (HA), comprising: introducing random mutations at a plurality of nucleotides in an isolated parental influenza virus nucleic acid molecule encoding an influenza B virus hemagglutinin (HA), thereby providing a library of influenza virus nucleic acid molecules encoding a mutant influenza B virus hemagglutinin, wherein the plurality of mutations in the influenza B virus hemagglutinin results in a plurality of amino acid substitutions or a deletion of a codon, at an amino acid position including positions that in one embodiment include position 40, 48, 56, 71, 73, 75, 76, 80, 81, 116, 117, 122, 126, 129, 133, 136, 137, 146, 148, 149, 150, 157, 162, 163, 164, 165, 167, 174, 197, 201, 202, 206, 208, 229, 232, 251, 254, 261, 266, 298, or 312, or any combination thereof (Yamagata mature HA numbering); introducing the library into cells so as to provide a library of cells that express the mutant hemagglutinins; and identifying a mutant hemagglutinin encoded by the library that is recognized by anti-Yamagata lineage specific sera and anti-Victoria lineage specific sera as a result of one or more substitutions and/or deletions at the one or more of the positions. As shown in FIG. 16, there are other positions than those listed above that vary between the lineages, e.g., positions 29, 34, 38, 40, 48, 56, 58, 71, 73, 75, 76, 80, 81, 89, 108, 116, 117, 121, 122, 125, 126, 129, 133, 136, 137, 139, 146, 148, 149, 150, 154, 162, 163, 164, 165, 166, 167, 168, 170, 172, 173, 175, 177, 179, 180, 182, 183, 184, 197, 198, 199, 201, 202, 203, 208, 209, 216, 220, 222, 229, 230, 232, 233, 235, 238, 251, 253, 254, 255, 256, 261, 262, 266, 267, 286, 298, 299, 312, 313, 368, 384, 415, 422, 479, 498, 505, 551, 555, 559, or any combination thereof. Thus, for any pairing of Yamagata and Victoria lineage parental HA sequences, the positions that vary may be employed to prepare ‘in between’ viruses. In one embodiment, the parental influenza virus nucleic acid molecule encodes a Yamagata lineage hemagglutinin. In one embodiment, the parental influenza virus nucleic acid molecule encodes a Victoria lineage hemagglutinin. In one embodiment, the mutant hemagglutinin has at up to 5, or 15 substitutions relative to the parent hemagglutinin. In one embodiment, the mutant hemagglutinin has at up to 15, 20 or 25 substitutions relative to the parent hemagglutinin. In one embodiment, the mutant hemagglutinin has at least one amino acid deletion relative to the parent hemagglutinin. In one embodiment, at least one deletion is at position 162 or 163. In one embodiment, the substituted amino acid is one of Y, H, R, E, D, K, M, V, T, I, A, Q, N, G, L, S, or P. In one embodiment, the mutant hemagglutinin has an amino acid at position 40 that is Y or H; position 48 that is R or E; position 56 that is D or K; position 71 that is M or K; position 73 that is V or T; position 75 that is T or K; position 76 that is T or I; position 80 that is K or R; position 81 that is A or V; position 116 that is K or H; position 117 that is I or V; position 122 that is Q or H; position 126 that is D or N; position 129 that is K or D; position 133 that is G or R; position 136 that is R or E; position 137 that is L or I; position 146 that is A or I; position 148 that is S or N; position 149 that is K or G; position 150 that is I or N; position 157 that is A or T; position 162 that is K or a deletion; position 163 that is D or a deletion; position 164 that is N or K; position 165 that is Y or N; position 167 that is N or T; position 174 that is V or I; position 197 that is K or E; position 201 that is K or A; position 202 that is S or K; position 206 that is D or Y; position 208 that is N or K; position 229 that is D or G; position 232 that is D or N; position 251 that is M or V; position 254 that is P or S; position 261 that is V or T; position 266 that is V or I; position 298 that is E or K; or position 312 that is K or E. In one embodiment, a majority of the substitutions in the mutant hemagglutinin are position 40 is Y; position 48 is R; position 56 is D; position 71 is M; position 73 is V; position 75 is T; position 76 is T; position 80 is K; position 81 is A; position 116 is K; position 117 is I; position 122 is Q; position 126 is D; position 129 is K; position 133 is G; position 136 is R; position 137 is L; position 146 is A; position 148 is S; position 149 is K; position 150 is I; position 162 is K; position 163 is D; position 164 is N; position 165 is; position 167 is N; position 174 is V; position 197 is K; position 201 is K; position 202 is S; position 208 is N; position 229 is D; position 232 is D; position 251 is M; position 254 is P; position 261 is V; position 266 is V; position 298 is E; or position 312 is K. In one embodiment, the mutant hemagglutinin has 16 to 24 substitutions selected from position 40 is Y; position 48 is R; position 56 is D; position 71 is M; position 73 is V; position 75 is T; position 76 is T; position 80 is K; position 81 is A; position 116 is K; position 117 is I; position 122 is Q; position 126 is D; position 129 is K; position 133 is G; position 136 is R; position 137 is L; position 146 is A; position 148 is S; position 149 is K; position 150 is I; position 162 is K; position 163 is D; position 164 is N; position 165 is; position 167 is N; position 174 is V; position 197 is K; position 201 is K; position 202 is S; position 208 is N; position 229 is D; position 232 is D; position 251 is M; position 254 is P; position 261 is V; position 266 is V; position 298 is E; or position 312 is K. In one embodiment, a majority of the substitutions in the mutant hemagglutinin are position 40 is H; position 48 is E; position 56 is K; position 71 is K; position 73 is T; position 75 is K; position 76 is I; position 80 is R; position 81 is V; position 116 is H; position 117 is V; position 122 is H; position 126 is N; position 129 is D; position 133 is R; position 136 is E; position 137 is I; position 146 is I; position 148 is N; position 149 is G; position 150 is N; position 162 is a deletion; position 163 is a deletion; position 164 is K; position 165 is N; position 167 is T; position 174 is I; position 197 is E; position 201 is A; position 202 is K; position 208 is K; position 229 is G; position 232 is N; position 251 is V; position 254 is S; position 261 is T; position 266 is I; position 298 is K; or position 312 is E. In one embodiment, the mutant hemagglutinin has 13 to 22 substitutions or deletions selected from of position 40 is H; position 48 is E; position 56 is K; position 71 is K; position 73 is T; position 75 is K; position 76 is I; position 80 is R; position 81 is V; position 116 is H; position 117 is V; position 122 is H; position 126 is N; position 129 is D; position 133 is R; position 136 is E; position 137 is I; position 146 is I; position 148 is N; position 149 is G; position 150 is N; position 162 is a deletion; position 163 is a deletion; position 164 is K; position 165 is N; position 167 is T; position 174 is I; position 197 is E; position 201 is A; position 202 is K; position 208 is K; position 229 is G; position 232 is N; position 251 is V; position 254 is S; position 261 is T; position 266 is I, position 298 is K; or position 312 is E. In one embodiment, the substitutions are at position 40, 48, 116, 126, 136, 137, 164, 197, 202, 208, 232, 251 or 261, or any combination thereof. In one embodiment, the substitutions are at position 56, 71, 73, 75, 76, 81, 146, 174, 201, 266, 298, 312, or the deletion is at position 162 or 163, or any combination thereof.

In one embodiment, the mutant hemagglutinin has an amino acid at position that is H; position 48 that is E; position 56 that is D; position 71 that is K; position 73 that is V; position 75 that is K; position 76 that is T; position 80 that is K or R; position 81 that is V; position 116 that is H; position 117 that is I or V; position 122 that is Q or H; position 126 that is D; position 129 that is K; position 133 that is G; position 136 that is R or E; position 137 that is I; position 146 that is A; position 149 that is K or G; position 148 that is S or N; position 150 that is I; position 162 that is K; position 163 that is D or as deletion; position 164 that is K; position 165 that is N; position 167 that is N; position 174 that is V or I; position 197 that is K or E; position 201 that is K or A; position 202 that is K; position 208 that is K; position 229 that is D; position 232 that is D or N; position 251 that is M or V; position 254 that is P; position 261 that is T; position 266 that is V or I; position 298 that is E; or position 312 that is K or E, or a combination thereof.

In one embodiment, the mutant hemagglutinin has an amino acid at position that is H; position 48 that is E; position 71 that is K; position 75 that is K; position 76 that is T; position 80 that is K or R; position 81 that is V; position 116 that is H; position 117 that is I or V; position 122 that is Q or H; position 129 that is K; position 133 that is G; position 136 that is R or E; position 137 that is I; position 149 that is K or G; position 148 that is S or N; position 162 that is K; position 163 that is D; position 164 that is K; position 165 that is N; position 167 that is N; position 174 that is V or I; position 201 that is K or A; position 202 that is K; position 208 that is K; position 229 that is D; position 251 that is M or V; position 254 that is P; position 261 that is T; position 298 that is E, or a combination thereof.

Also provided is a method to prepare an influenza B virus encoding a mutant hemagglutinin that is recognized by anti-Yamagata lineage specific sera and anti-Victoria lineage specific sera relative to a parental influenza B virus hemagglutinin, comprising: introducing a mutation in a parental influenza B virus HA nucleic acid molecule at two or more codons for an amino acid at position 29, 34, 38, 40, 48, 56, 58, 71, 73, 75, 76, 80, 81, 89, 108, 116, 117, 121, 122, 125, 126, 129, 133, 136, 137, 139, 146, 148, 149, 150, 154, 162, 163, 164, 165, 166, 167, 168, 170, 172, 173, 175, 177, 179, 180, 182, 183, 184, 197, 198, 199, 201, 202, 203, 208, 209, 216, 220, 222, 229, 230, 232, 233, 235, 238, 251, 253, 254, 255, 256, 261, 262, 266, 267, 286, 298, 299, 312, 313, 368, 384, 415, 422, 479, 498, 505, 551, 555, 559, or any combination thereof, e.g., 40, 48, 56, 71, 73, 75, 76, 80, 81, 116, 117, 122, 126, 129, 133, 136, 137, 146, 148, 149, 150, 162, 163, 164, 165, 167, 174, 197, 201, 202, 208, 229, 232, 251, 254, 261, 266, 298, or 312, or any combination thereof, thereby providing a mutated HA nucleic acid molecule; and preparing one or more influenza B viruses or isolated HA with the mutated HA nucleic acid molecule. In one embodiment, the parent hemagglutinin is a Yamagata lineage hemagglutinin. In one embodiment, the parent hemagglutinin is a Victoria lineage hemagglutinin. In one embodiment, the mutant hemagglutinin has at up to 5, 10 or 15 substitutions relative to the parent hemagglutinin. In one embodiment, the mutant hemagglutinin has at up to 15, 20 or substitutions relative to the parent hemagglutinin. In one embodiment, the mutant hemagglutinin has at least one amino acid deletion relative to the parent hemagglutinin. In one embodiment, at least one deletion is at position 162 or 163. In one embodiment, the mutant hemagglutinin has an amino acid at position 40 that is Y or H; position 48 that is R or E; position 56 that is D or K; position 71 that is M or K; position 73 that is V or T; position 75 that is T or K; position 76 that is T or I; position 80 that is K or R; position 81 that is A or V; position 116 that is K or H; position 117 that is I or V; position 122 that is Q or H; position 126 that is D or N; position 129 that is K or D; position 133 that is G or R; position 136 that is R or E; position 137 that is L or I; position 146 that is A or I; position 148 that is S or N; position 149 that is K or G; position 150 that is I or N; position 162 that is K or a deletion; position 163 that is D or a deletion; position 164 that is N or K; position 165 that is Y or N; position 167 that is N or T; position 174 that is V or I; position 197 that is K or E; position 201 that is K or A; position 202 that is S or K; position 208 that is N or K; position 229 that is D or G; position 232 that is D or N; position 251 that is M or V; position 254 that is P or S; position 261 that is V or T; position 266 that is V or I; position 298 that is E or K; or position 312 that is K or E. In one embodiment, a majority of the substitutions in the mutant hemagglutinin are position 40 is Y; position 48 is R; position 56 is D; position 71 is M; position 73 is V; position 75 is T; position 76 is T; position 80 is K; position 81 is A; position 116 is K; position 117 is I; position 122 is Q; position 126 is D; position 129 is K; position 133 is G; position 136 is R; position 137 is L; position 146 is A; position 148 is S; position 149 is K; position 150 is I; position 162 is K; position 163 is D; position 164 is N; position 165 is; position 167 is N; position 174 is V; position 197 is K; position 201 is K; position 202 is S; position 208 is N; position 229 is D; position 232 is D; position 251 is M; position 254 is P; position 261 is V; position 266 is V; position 298 is E; or position 312 is K. In one embodiment, the mutant hemagglutinin has 16 to 24 substitutions selected from position 40 is Y; position 48 is R; position 56 is D; position 71 is M; position 73 is V; position 75 is T; position 76 is T; position 80 is K; position 81 is A; position 116 is K; position 117 is I; position 122 is Q; position 126 is D; position 129 is K; position 133 is G; position 136 is R; position 137 is L; position 146 is A; position 148 is S; position 149 is K; position 150 is I; position 162 is K; position 163 is D; position 164 is N; position 165 is; position 167 is N; position 174 is V; position 197 is K; position 201 is K; position 202 is S; position 208 is N; position 229 is D; position 232 is D; position 251 is M; position 254 is P; position 261 is V; position 266 is V; position 298 is E; or position 312 is K. In one embodiment, a majority of the substitutions in the mutant hemagglutinin are position 40 is H; position 48 is E; position 56 is K; position 71 is K; position 73 is T; position 75 is K; position 76 is I; position 80 is R; position 81 is V; position 116 is H; position 117 is V; position 122 is H; position 126 is N; position 129 is D; position 133 is R; position 136 is E; position 137 is I; position 146 is I; position 148 is N; position 149 is G; position 150 is N; position 162 is a deletion; position 163 is a deletion; position 164 is K; position 165 is N; position 167 is T; position 174 is I; position 197 is E; position 201 is A; position 202 is K; position 208 is K; position 229 is G; position 232 is N; position 251 is V; position 254 is S; position 261 is T; position 266 is I; position 298 is K; or position 312 is E. In one embodiment, the mutant hemagglutinin has 13 to 22 substitutions or deletions selected from of position 40 is H; position 48 is E; position 56 is K; position 71 is K; position 73 is T; position 75 is K; position 76 is 1; position 80 is R; position 81 is V; position 116 is H; position 117 is V; position 122 is H; position 126 is N; position 129 is D; position 133 is R; position 136 is E; position 137 is 1; position 146 is I; position 148 is N; position 149 is G; position 150 is N; position 162 is a deletion; position 163 is a deletion; position 164 is K; position 165 is N; position 167 is T; position 174 is I; position 197 is E; position 201 is A; position 202 is K; position 208 is K; position 229 is G; position 232 is N; position 251 is V; position 254 is S; position 261 is T; position 266 is I; position 298 is K; or position 312 is E. In one embodiment, the substitutions are at position 40, 48, 116, 126, 136, 137, 164, 197, 202, 208, 232, 251 or 261, or any combination thereof. In one embodiment, the substitutions are at position 56, 71, 73, 75, 76, 81, 146, 174, 201, 266, 298, 312, or the deletion is at position 162 or 163, or any combination thereof.

Further provided is a composition comprising a recombinant influenza B virus encoding a hemagglutinin comprising a plurality of mutations relative to a parent virus hemagglutinin, wherein the recombinant influenza B virus HA comprises one or more substitutions at position 29, 34, 38, 40, 48, 56, 58, 71, 73, 75, 76, 80, 81, 89, 108, 116, 117, 121, 122, 125, 126, 129, 133, 136, 137, 139, 146, 148, 149, 150, 154, 162, 163, 164, 165, 166, 167, 168, 170, 172, 173, 175, 177, 179, 180, 182, 183, 184, 197, 198, 199, 201, 202, 203, 208, 209, 216, 220, 222, 229, 230, 232, 233, 235, 238, 251, 253, 254, 255, 256, 261, 262, 266, 267, 286, 298, 299, 312, 313, 368, 384, 415, 422, 479, 498, 505, 551, 555, 559, or any combination thereof, e.g., position 40, 48, 56, 71, 73, 75, 76, 80, 81, 116, 117, 122, 126, 129, 133, 136, 137, 146, 148, 149, 150, 162, 165, 167, 174, 197, 201, 202, 208, 229, 232, 251, 254, 261, 266, 298, or 312, or comprises one or more deletions at position 163 or 164, or any combination thereof, relative to a Yamagata lineage hemagglutinin or a Victoria lineage hemagglutinin. In one embodiment, the parent hemagglutinin is a Yamagata lineage hemagglutinin. In one embodiment, the parent hemagglutinin is a Victoria lineage hemagglutinin. In one embodiment, the mutant hemagglutinin has at up to 5, 10 or 15 substitutions relative to the parent hemagglutinin. In one embodiment, the mutant hemagglutinin has at up to 15, 20 or 25 substitutions relative to the parent hemagglutinin. In one embodiment, the mutant hemagglutinin has at least one amino acid deletion relative to the parent hemagglutinin. In one embodiment, the at least one deletion is at position 162 or 163. In one embodiment, the mutant hemagglutinin has an amino acid at position 40 that is Y or H; position 48 that is R or E; position 56 that is D or K; position 71 that is M or K; position 73 that is V or T; position 75 that is T or K; position 76 that is T or I; position 80 that is K or R; position 81 that is A or V; position 116 that is K or H; position 117 that is I or V; position 122 that is Q or H; position 126 that is D or N; position 129 that is K or D; position 133 that is G or R; position 136 that is R or E; position 137 that is L or I; position 146 that is A or I; position 148 that is S or N; position 149 that is K or G; position 150 that is I or N; position 162 that is K or a deletion; position 163 that is D or a deletion; position 164 that is N or K; position 165 that is Y or N; position 167 that is N or T; position 174 that is V or I; position 197 that is K or E; position 201 that is K or A; position 202 that is S or K; position 208 that is N or K; position 229 that is D or G; position 232 that is D or N; position 251 that is M or V; position 254 that is P or S; position 261 that is V or T; position 266 that is V or I; position 298 that is E or K; or position 312 that is K or E. In one embodiment, a majority of the substitutions in the mutant virus are position 40 is Y; position 48 is R; position 56 is D; position 71 is M; position 73 is V; position 75 is T; position 76 is T; position 80 is K; position 81 is A; position 116 is K; position 117 is I; position 122 is Q; position 126 is D; position 129 is K; position 133 is G; position 136 is R; position 137 is L; position 146 is A; position 148 is S; position 149 is K; position 150 is I; position 162 is K; position 163 is D; position 164 is N; position 165 is; position 167 is N; position 174 is V; position 197 is K; position 201 is K; position 202 is S; position 208 is N; position 229 is D; position 232 is D; position 251 is M; position 254 is P; position 261 is V; position 266 is V; position 298 is E; or position 312 is K. In one embodiment, the mutant virus has 16 to 24 substitutions selected from position 40 is Y; position 48 is R; position 56 is D; position 71 is M; position 73 is V; position 75 is T; position 76 is T; position 80 is K; position 81 is A; position 116 is K; position 117 is I; position 122 is Q; position 126 is D; position 129 is K; position 133 is G; position 136 is R; position 137 is L; position 146 is A; position 148 is S; position 149 is K; position 150 is I; position 162 is K; position 163 is D; position 164 is N; position 165 is; position 167 is N; position 174 is V; position 197 is K; position 201 is K; position 202 is S; position 208 is N; position 229 is D; position 232 is D; position 251 is M; position 254 is P; position 261 is V; position 266 is V; position 298 is E; or position 312 is K. In one embodiment, a majority of the substitutions in the mutant virus are position 40 is H; position 48 is E; position 56 is K; position 71 is K; position 73 is T; position 75 is K; position 76 is I; position 80 is R; position 81 is V; position 116 is H; position 117 is V; position 122 is H; position 126 is N; position 129 is D; position 133 is R; position 136 is E; position 137 is I; position 146 is I; position 148 is N; position 149 is G; position 150 is N; position 162 is a deletion; position 163 is a deletion; position 164 is K; position 165 is N; position 167 is T; position 174 is I; position 197 is E; position 201 is A; position 202 is K; position 208 is K; position 229 is G; position 232 is N; position 251 is V; position 254 is S; position 261 is T; position 266 is I; position 298 is K; or position 312 is E. In one embodiment, the mutant virus has 13 to 22 substitutions or deletions selected from of position 40 is H; position 48 is E; position 56 is K; position 71 is K; position 73 is T; position 75 is K; position 76 is I; position 80 is R; position 81 is V; position 116 is H; position 117 is V; position 122 is H; position 126 is N; position 129 is D; position 133 is R; position 136 is E; position 137 is I; position 146 is 1; position 148 is N; position 149 is G; position 150 is N; position 162 is a deletion; position 163 is a deletion; position 164 is K; position 165 is N; position 167 is T; position 174 is 1; position 197 is E; position 201 is A; position 202 is K; position 208 is K; position 229 is G; position 232 is N; position 251 is V; position 254 is S; position 261 is T; position 266 is 1; position 298 is K; or position 312 is E. In one embodiment, the substitutions are at position 40, 48, 116, 126, 136, 137, 164, 197, 202, 208, 232, 251 or 261, or any combination thereof. In one embodiment, the substitutions are at position 56, 71, 73, 75, 76, 81, 146, 174, 201, 266, 298, 312, or the deletion is at position 162 or 163, or any combination thereof. In one embodiment, the composition further comprises one or more other influenza viruses or one or more antigens thereof. In one embodiment, the one or more other influenza viruses comprise one or more influenza A viruses. In one embodiment, at least one of the positions include when position 40 is H; position 48 is E; position 56 is K; position 71 is K; position 73 is T; or position 75 is K. In one embodiment, at least two of the positions include when position 76 is I; 80 is R; 81 is V; 116 is H; 117 is V; 122 is H; or 126 is N. In one embodiment, at least four of the positions include when position 129 is D; 133 is R; 136 is E; 137 is I; 146 is I; 148 is N; 149 is G; 150 is N; 162 is a deletion; or 163 is deletion. In one embodiment, at least five of the positions include when position 164 is K; 165 is N; 167 is T; 174 is I; 197 is E; 201 is A, 202 is K; 208 is K; 229 is G; 232 is N; 251 is V; 254 is S; 261 is T; 266 is I; 298 is K; or 312 is E. In one embodiment, the virus does not bind to sera specific for one of SEQ ID Nos. 1-3. In one embodiment, the virus binds to sera specific for SEQ ID NO:4 and SEQ ID NO:5.

In one embodiment, the composition comprises or encodes a mutant hemagglutinin that has an amino acid at position 40 that is H; position 48 that is E; position 56 that is D; position 71 that is K; position 73 that is V; position 75 that is K; position 76 that is T; position 80 that is K or R; position 81 that is V; position 116 that is H; position 117 that is I or V; position 122 that is Q or H; position 126 that is D; position 129 that is K; position 133 that is G; position 136 that is R or E; position 137 that is I; position 146 that is A; position 149 that is K or G; position 148 that is S or N; position 150 that is I; position 162 that is K; position 163 that is D or as deletion; position 164 that is K; position 165 that is N; position 167 that is N; position 174 that is V or I; position 197 that is K or E; position 201 that is K or A; position 202 that is K; position 208 that is K; position 229 that is D; position 232 that is D or N; position 251 that is M or V; position 254 that is P; position 261 that is T; position 266 that is V or I; position 298 that is E; or position 312 that is K or E, or a combination thereof. In one embodiment, the composition comprises liposomes, e.g., lipid nanoparticles comprising nucleic acid encoding the mutant hemagglutinin.

In one embodiment, the composition comprises or encodes a mutant hemagglutinin that has an amino acid at position 40 that is H; position 48 that is E; position 71 that is K; position 75 that is K; position 76 that is T; position 80 that is K or R; position 81 that is V; position 116 that is H; position 117 that is I or V; position 122 that is Q or H; position 129 that is K; position 133 that is G; position 136 that is R or E; position 137 that is I; position 149 that is K or G; position 148 that is S or N; position 162 that is K; position 163 that is D; position 164 that is K; position 165 that is N; position 167 that is N; position 174 that is V or I; position 201 that is K or A; position 202 that is K; position 208 that is K; position 229 that is D; position 251 that is M or V; position 254 that is P; position 261 that is T; position 298 that is E, or a combination thereof. In one embodiment, the composition comprises liposomes, e.g., lipid nanoparticles comprising nucleic acid encoding the mutant hemagglutinin.

In one embodiment, an isolated virus having a plurality of the substitutions and/or deletion(s) is provided. In one embodiment, a pharmaceutical composition comprising an effective amount of the ‘in between’ virus is provided, and optionally further comprising an adjuvant and/or a pharmaceutically acceptable carrier. In one embodiment, the composition is suitable for intranasal or subcutaneous administration.

Further provided is a method to immunize an animal, comprising: administering an effective amount of a composition comprising an ‘in between’ influenza B virus to an animal.

In one embodiment, an isolated nucleic acid molecule is provided comprising an open reading frame encoding an influenza B virus HA having a first amino acid and a second amino acid or at least one deletion, at position 40 that is Y or H; 48 that is R or E; 56 that is D or K; 71 that is M or K; 73 that is V or T; 75 that is T or K; 76 that is T or I; 80 that is K or R; 81 that is A or V; 116 that is K or H; 117 that is I or V; 122 that is Q or H; 126 that is D or N; 129 that is K or D; 133 that is G or R; 136 that is R or E; 137 that is L or I; 146 that is A or I; 148 that is S or N; 149 that is K or G; 150 that is I or N; 162 that is K or deletion; 163 that is D or deletion; 164 that is N or K; 165 that is Y or N; 167 that is N or T; 174 that is V or I; 197 that is K or E; 201 that is K or A; 202 that is S or K; 208 that is N or K; 229 that is D or G; 232 that is D or N; 251 that is M or V; 254 that is P or S; 261 that is V or T; 266 that is V or I; 298 that is E or K; or 312 that is K or E, or any combination thereof.

### BRIEF DESCRIPTION OF THE FIGURES

FIG. 1. Hypothetical antigenic map of IBVs and IBV mutants. Viruses of the Yamagata- and Victoria-lineages are shown in dark blue and brown, respectively. Hypothetical mutants based on a Victoria lineage or a Yamagata lineage virus are shown in light blue or gold, respectively. Mutants located in-between the two lineages are attractive vaccine candidates.

FIG. 2. Current antigenic map of influenza B viruses. Ancestral viruses are shown in red. Reference viruses of the Yamagata- and Victoria-lineages are shown in purple and dark green, respectively. Compared with the parental viruses, some of the B/Phuket/3073/2013 (Yamagata lineage)-derived mutants (light blue) moved towards the Victoria-lineage viruses, while many of the B/Washington/02/2019 (Victoria lineage)-derived mutants (light green) moved towards the Yamagata lineage.

FIG. 3. Three-dimensional structure of IBV HA (PDB #4FQM). The three monomers are shown in gray, light blue, and green. Amino acids that differ between the B/Florida/78/2015 (Victoria lineage) and B/Phuket/3073/2013 (Yamagata lineage) HA1 subunits are shown in red. The receptor-binding pocket is shown in yellow.

FIG. 4. Significance of immunogenicity. Small, solid circles indicate antigens; small, solid squares indicate sera; large, transparent circles indicate the ‘range’ of reactivity of the serum. The Yamagata- and Victoria-lineages are shown in dark blue and brown, respectively; the IBV vaccine candidate is shown in gold. If our IBV HA vaccine candidate is poorly immunogenic (left panel), the low antibody titers of the serum will not allow reactivity with antigens located at a greater distance (such as the Yamagata- and Victoria-lineage viruses shown in dark blue and brown, respectively). However, an IBV HA vaccine candidate with high immunogenicity (right panel) will elicit high antibody titers in the serum, allowing reactivity with IBVs of both lineages. Each grid unit in an antigenic map equals a two-fold difference in HI titers.

FIG. 5. Comparison of three different adjuvants. Ferrets were mock-vaccinated (No vac) or sequentially vaccinated with recombinant HAs representing a human H3N2 virus from the FU02- or a PE09-antigenic cluster, respectively. Three weeks later, ferret sera were tested for neutralizing antibody titers against human H3N2 viruses from the WU95-, FU02-, PE09-, and PE09 antigenic clusters. The numbers on the X-axis indicate individual ferrets.

FIG. 6. Schematic diagram of the IBV HA phylogenetic tree. Shown are the ancestral viruses, and the Victoria- and Yamagata-lineages with the viruses after which the lineages were named.

FIG. 7. Exemplary ancestral IBV sequence (B/Johannesburg/1958) (SEQ ID NO:1).

FIG. 8. Exemplary ancestral IBV sequence (B/Czechoslovakia/1/1949) (SEQ ID NO:2).

FIG. 9. Exemplary ancestral IBV sequence B/Massachusetts/3/1966) (SEQ ID NO:3).

FIG. 10. Flu B antigenic map. Preliminary antigenic map of influenza B viruses. Ancestral viruses are shown in red. Reference viruses of the Yamagata- and Victoria-lineages are shown in purple and dark green, respectively. Compared with the parental viruses, some of the B/Phuket/3073/2013 (Yamagata lineage)-derived mutants (light blue) moved towards the Victoria-lineage viruses, while many of the B/Washington/02/2019 (Victoria lineage)-derived mutants (light green) moved towards the Yamagata lineage. Red arrows indicate the mutants selected to generate ferret sera.

FIG. 11. Amino acid alignment of exemplary IBV HA from the Yamagata lineage and the Victoria lineage (“Phuket” and “Washington” disclosed herein).

FIG. 12. Exemplary ‘in between’ IBV HAs.

FIG. 13. Sequences for the HA of B/Phuket/3073/2013 (SEQ ID NO:4), B/Florida/78/2015 (SEQ ID NO:5), and B/Washington/02/2019 (SEQ ID NO:6).

FIG. 14. Exemplary ferret sera including ‘in between’ sera that react with viruses of both lineages. Shown is the reactivity to parental virus, ancestral virus, Yamagata lineage virus and Victoria lineage virus. Four sera show substantial reactivity with viruses of both lineages (titers of 160 or higher are highlighted in yellow; note that a titer of 40 is considered protective in humans).

FIG. 15. Amino acid differences in three parental strains.

FIGS. 16 and 16A-16V. Amino acid comparison of selected ancestral, Yamagata-lineage, and Victoria-lineage influenza B virus HA proteins (SEQ ID NOs: 11-22).

FIG. 17. Exemplary ‘in between’ IBV HAs.

FIG. 18. Challenge experiments to determine if vaccination with mutants protect better than vaccination with heterologous virus. PPW76 has Phuket HA1, Phuket HA2 and Washington NA; WWP-29 has Washington HA1, Washington HA2 and Phuket NA. Animals were vaccinated and challenged with the indicated recomb. HA with Alum. Animals were then challenged with Washington/02 or Phuket/3073 virus. Shown are the virus titers in nasal swabs.

### DETAILED DESCRIPTION

Current influenza B vaccines are lineage-specific, i.e., they do not provide efficient protection against influenza B viruses from the other lineages. The present viruses may result in vaccines that provide protection against influenza B viruses from both lineages. In particular, the influenza B viruses circulating in humans fall into two genetically and antigenically distinct lineages, i.e., the Victoria- and Yamagata-lineages. Vaccines that protect against viruses from one lineage do not provide efficient protection against viruses from the other lineages. As a consequence, many influenza vaccines are now quadrivalent, i.e., they are composed of four different vaccine strains representing influenza A/H3N2 and A/H1N1 viruses, and both influenza B virus lineages. Efforts are therefore underway to develop broadly protective vaccines that protect against influenza B viruses of both lineages. Using a novel approach, we have developed mutant influenza B viruses that react with sera specific to the Yamagata- or Victoria-lineage, respective; thus, these mutants are antigenically located ‘between’ the two lineages. The use of such mutants as vaccine viruses may elicit antibodies that provide protection against influenza B viruses from both lineages.

### Definitions

As used herein, the term “isolated” refers to in vitro preparation and/or isolation of a nucleic acid molecule, e.g., vector or plasmid, peptide or polypeptide (protein), or virus, so that it is not associated with in vivo substances, or is substantially purified from in vitro substances. An isolated virus preparation is generally obtained by in vitro culture and propagation, and/or via passage in eggs, and is substantially free from other infectious agents.

As used herein, “substantially purified” means the object species is the predominant species, e.g., on a molar basis it is more abundant than any other individual species in a composition, and preferably is at least about 80% of the species present, and optionally 90% or greater, e.g., 95%, 98%, 99% or more, of the species present in the composition.

As used herein, “substantially free” means below the level of detection for a particular infectious agent using standard detection methods for that agent.

A “recombinant” virus is one which has been manipulated in vitro, e.g., using recombinant DNA techniques, to introduce changes to the viral genome. Reassortant viruses can be prepared by recombinant or nonrecombinant techniques.

As used herein, the term “recombinant nucleic acid” or “recombinant DNA sequence or segment” refers to a nucleic acid, e.g., to DNA, that has been derived or isolated from a source, that may be subsequently chemically altered in vitro, so that its sequence is not naturally occurring, or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, by the methodology of genetic engineering.

As used herein, a “heterologous” influenza virus gene or viral segment is from an influenza virus source that is different than a majority of the other influenza viral genes or viral segments in a recombinant, e.g., reassortant, influenza virus.

The terms “isolated polypeptide”, “isolated peptide” or “isolated protein” include a polypeptide, peptide or protein encoded by cDNA or recombinant RNA including one of synthetic origin, or some combination thereof.

The term “recombinant protein” or “recombinant polypeptide” as used herein refers to a protein molecule expressed from a recombinant DNA molecule. In contrast, the term “native protein” is used herein to indicate a protein isolated from a naturally occurring (i.e., a nonrecombinant) source. Molecular biological techniques may be used to produce a recombinant form of a protein with identical properties as compared to the native form of the protein.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Alignments using these programs can be performed using the default parameters. Software for performing BLAST analyses is publicly available through the National Center for Biotechnology Information (http://www.ncbi.nlm.nih.gov/). The algorithm may involve first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm may also perform a statistical analysis of the similarity between two sequences. One measure of similarity provided by the BLAST algorithm may be the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

The BLASTN program (for nucleotide sequences) may use as defaults a wordlength (W) of 11, an expectation (E) of 10, a cutoff of 100, M=5, N=−4, and a comparison of both strands. For amino acid sequences, the BLASTP program may use as defaults a wordlength (W) of 3, an expectation (E) of 10, and the BLOSUM62 scoring matrix. See http://www.ncbi.nlm.nih.gov. Alignment may also be performed manually by inspection.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

### Influenza Vaccines

A vaccine includes at least one of the isolated recombinant influenza viruses having the desired property, e.g., induces sera that recognize influenza B viruses from both Yamagata and Victoria lineages, as well as maintaining the structural and functional integrity of HA, and optionally one or more other isolated viruses including other isolated influenza viruses having a desired property, one or more immunogenic proteins or glycoproteins of one or more isolated influenza viruses or one or more other pathogens, e.g., an immunogenic protein from one or more bacteria, non-influenza viruses, yeast or fungi, or isolated nucleic acid encoding one or more viral proteins (e.g., DNA vaccines) including one or more immunogenic proteins of the isolated influenza virus. In one embodiment, the influenza viruses may be vaccine vectors for influenza virus or other pathogens.

A complete virion vaccine may be concentrated by ultrafiltration and then purified by zonal centrifugation or by chromatography. Viruses other than the virus of the disclosure, such as those included in a multivalent vaccine, may be inactivated before or after purification using formalin or beta-propiolactone, for instance.

A subunit vaccine comprises purified glycoproteins. Such a vaccine may be prepared as follows: using viral suspensions fragmented by treatment with detergent, the surface antigens are purified, by ultracentrifugation for example. The subunit vaccines thus contain mainly HA protein, and also NA. The detergent used may be cationic detergent for example, such as hexadecyl trimethyl ammonium bromide (Bachmeyer, 1975), an anionic detergent such as ammonium deoxycholate (Laver & Webster, 1976); or a nonionic detergent such as that commercialized under the name TRITON X100. The hemagglutinin may also be isolated after treatment of the virions with a protease such as bromelin, and then purified. The subunit vaccine may be combined with an attenuated virus of the disclosure in a multivalent vaccine.

A split vaccine comprises virions which have been subjected to treatment with agents that dissolve lipids. A split vaccine can be prepared as follows: an aqueous suspension of the purified virus obtained as above, inactivated or not, is treated, under stirring, by lipid solvents such as ethyl ether or chloroform, associated with detergents. The dissolution of the viral envelope lipids results in fragmentation of the viral particles. The aqueous phase is recuperated containing the split vaccine, constituted mainly of hemagglutinin and neuraminidase with their original lipid environment removed, and the core or its degradation products. Then the residual infectious particles are inactivated if this has not already been done. The split vaccine may be combined with an attenuated virus of the disclosure in a multivalent vaccine.

Inactivated Vaccines. Inactivated influenza virus vaccines are provided by inactivating replicated virus using known methods, such as, but not limited to, formalin or β-propiolactone treatment. Inactivated vaccine types that can be used in the disclosure can include whole-virus (WV) vaccines or subvirion (SV) (split) vaccines. The WV vaccine contains intact, inactivated virus, while the SV vaccine contains purified virus disrupted with detergents that solubilize the lipid-containing viral envelope, followed by chemical inactivation of residual virus.

In addition, vaccines that can be used include those containing the isolated HA and NA surface proteins, which are referred to as surface antigen or subunit vaccines.

Live Attenuated Virus Vaccines. Live, attenuated influenza virus vaccines, such as those including a recombinant virus of the disclosure can be used for preventing or treating influenza virus infection. Attenuation may be achieved in a single step by transfer of attenuated genes from an attenuated donor virus to a replicated isolate or reassorted virus according to known methods. Since resistance to influenza virus is mediated primarily by the development of an immune response to the HA and/or NA glycoproteins, the genes coding for these surface antigens come from the reassorted viruses or clinical isolates. The attenuated genes are derived from an attenuated parent. In this approach, genes that confer attenuation generally do not code for the HA and NA glycoproteins.

Viruses (donor influenza viruses) are available that are capable of reproducibly attenuating influenza viruses, e.g., a cold adapted (ca) donor virus can be used for attenuated vaccine production. Live, attenuated reassortant virus vaccines can be generated by mating the ca donor virus with a virulent replicated virus. Reassortant progeny are then selected at 25° C. (restrictive for replication of virulent virus), in the presence of an appropriate antiserum, which inhibits replication of the viruses bearing the surface antigens of the attenuated ca donor virus. Useful reassortants are: (a) infectious, (b) attenuated for seronegative non-adult mammals and immunologically primed adult mammals, (c) immunogenic and (d) genetically stable. The immunogenicity of the ca reassortants parallels their level of replication. Thus, the acquisition of the six transferable genes of the ca donor virus by new wild-type viruses has reproducibly attenuated these viruses for use in vaccinating susceptible mammals both adults and non-adult.

Other attenuating mutations can be introduced into influenza virus genes by site-directed mutagenesis to rescue infectious viruses bearing these mutant genes. Attenuating mutations can be introduced into non-coding regions of the genome, as well as into coding regions. Such attenuating mutations can also be introduced into genes other than the HA or NA, e.g., the PB2 polymerase gene. Thus, new donor viruses can also be generated bearing attenuating mutations introduced by site-directed mutagenesis, and such new donor viruses can be used in the production of live attenuated reassortants vaccine candidates in a manner analogous to that described above for the ca donor virus. Similarly, other known and suitable attenuated donor strains can be reassorted with influenza virus to obtain attenuated vaccines suitable for use in the vaccination of mammals.

In one embodiment, such attenuated viruses maintain the genes from the virus that encode antigenic determinants substantially similar to those of the original clinical isolates. This is because the purpose of the attenuated vaccine is to provide substantially the same antigenicity as the original clinical isolate of the virus, while at the same time lacking pathogenicity to the degree that the vaccine causes minimal chance of inducing a serious disease condition in the vaccinated mammal.

The viruses in a multivalent vaccine can thus be attenuated or inactivated, formulated and administered, according to known methods, as a vaccine to induce an immune response in an animal, e.g., a mammal. Methods are well-known in the art for determining whether such attenuated or inactivated vaccines have maintained similar antigenicity to that of the clinical isolate or high growth strain derived therefrom. Such known methods include the use of antisera or antibodies to eliminate viruses expressing antigenic determinants of the donor virus; chemical selection (e.g., amantadine or rimantidine); HA and NA activity and inhibition; and nucleic acid screening (such as probe hybridization or PCR) to confirm that donor genes encoding the antigenic determinants (e.g., HA or NA genes) are not present in the attenuated viruses.

### Pharmaceutical Compositions

Pharmaceutical compositions, suitable for inoculation, e.g., nasal, parenteral or oral administration, comprise one or more influenza virus isolates, e.g., one or more attenuated or inactivated influenza viruses, a subunit thereof, isolated protein(s) thereof, and/or isolated nucleic acid encoding one or more proteins thereof, optionally further comprising sterile aqueous or non-aqueous solutions, suspensions, and emulsions. The compositions can further comprise auxiliary agents or excipients, as known in the art. The composition of the disclosure is generally presented in the form of individual doses (unit doses).

Conventional vaccines generally contain about 0.1 to 200 μg, e.g., 30 to 100 μg, of HA from each of the strains entering into their composition. The vaccine forming the main constituent of the vaccine composition of the disclosure may comprise a single influenza virus, or a combination of influenza viruses, for example, at least two or three influenza viruses, including one or more reassortant(s).

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

When a composition is used for administration to an individual, it can further comprise salts, buffers, adjuvants, or other substances which are desirable for improving the efficacy of the composition. For vaccines, adjuvants, substances which can augment a specific immune response, can be used. Normally, the adjuvant and the composition are mixed prior to presentation to the immune system, or presented separately, but into the same site of the organism being immunized.

Heterogeneity in a vaccine may be provided by mixing replicated influenza viruses for at least two influenza virus strains, such as 2-20 strains or any range or value therein. Vaccines can be provided for variations in a single strain of an influenza virus, using techniques known in the art.

A pharmaceutical composition according to the present disclosure may further or additionally comprise at least one chemotherapeutic compound, for example, for gene therapy, immunosuppressants, anti-inflammatory agents or immune enhancers, and for vaccines, chemotherapeutics including, but not limited to, gamma globulin, amantadine, guanidine, hydroxybenzimidazole, interferon-α, interferon-β, interferon-γ, tumor necrosis factor-alpha, thiosemicarbarzones, methisazone, rifampin, ribavirin, a pyrimidine analog, a purine analog, foscamet, phosphonoacetic acid, acyclovir, dideoxynucleosides, a protease inhibitor, or ganciclovir.

The composition can also contain variable but small quantities of endotoxin-free formaldehyde, and preservatives, which have been found safe and not contributing to undesirable effects in the organism to which the composition is administered.

### Pharmaceutical Purposes

The administration of the composition (or the antisera that it elicits) may be for either a “prophylactic” or “therapeutic” purpose. When provided prophylactically, the compositions of the disclosure which are vaccines are provided before any symptom or clinical sign of a pathogen infection becomes manifest. The prophylactic administration of the composition serves to prevent or attenuate any subsequent infection. When provided prophylactically, the gene therapy compositions of the disclosure, are provided before any symptom or clinical sign of a disease becomes manifest. The prophylactic administration of the composition serves to prevent or attenuate one or more symptoms or clinical signs associated with the disease.

When provided therapeutically, a viral vaccine is provided upon the detection of a symptom or clinical sign of actual infection. The therapeutic administration of the compound(s) serves to attenuate any actual infection. When provided therapeutically, a gene therapy composition is provided upon the detection of a symptom or clinical sign of the disease. The therapeutic administration of the compound(s) serves to attenuate a symptom or clinical sign of that disease.

Thus, a vaccine composition may be provided either before the onset of infection (so as to prevent or attenuate an anticipated infection) or after the initiation of an actual infection. Similarly, for gene therapy, the composition may be provided before any symptom or clinical sign of a disorder or disease is manifested or after one or more symptoms are detected.

A composition is said to be “pharmacologically acceptable” if its administration can be tolerated by a recipient mammal. Such an agent is said to be administered in a “therapeutically effective amount” if the amount administered is physiologically significant. A composition of the present disclosure is physiologically significant if its presence results in a detectable change in the physiology of a recipient patient, e.g., enhances at least one primary or secondary humoral or cellular immune response against at least one strain of an infectious influenza virus.

The “protection” provided need not be absolute, i.e., the influenza infection need not be totally prevented or eradicated, if there is a statistically significant improvement compared with a control population or set of mammals. Protection may be limited to mitigating the severity or rapidity of onset of symptoms or clinical signs of the influenza virus infection.

### Pharmaceutical Administration

A composition having one of more influenza viruses with the desired properties may confer resistance to one or more pathogens, e.g., one or more influenza virus strains, by either passive immunization or active immunization. In active immunization, an attenuated live vaccine composition is administered prophylactically to a host (e.g., a mammal), and the host's immune response to the administration protects against infection and/or disease. For passive immunization, the elicited antisera can be recovered and administered to a recipient suspected of having an infection caused by at least one influenza virus strain.

In one embodiment, the vaccine is provided to a mammalian female (at or prior to pregnancy or parturition), under conditions of time and amount sufficient to cause the production of an immune response which serves to protect both the female and the fetus or newborn (via passive incorporation of the antibodies across the placenta or in the mother's milk).

The present disclosure thus includes methods for preventing or attenuating a disorder or disease, e.g., an infection by at least one strain of pathogen. As used herein, a vaccine is said to prevent or attenuate a disease if its administration results either in the total or partial attenuation (i.e., suppression) of a clinical sign or condition of the disease, or in the total or partial immunity of the individual to the disease. As used herein, a gene therapy composition is said to prevent or attenuate a disease if its administration results either in the total or partial attenuation (i.e., suppression) of a clinical sign or condition of the disease, or in the total or partial immunity of the individual to the disease.

A composition having at least one influenza virus of the present disclosure, including one or more other isolated viruses, one or more isolated viral proteins thereof, one or more isolated nucleic acid molecules encoding one or more viral proteins thereof, or a combination thereof, may be administered by any means that achieve the intended purposes.

For example, administration of such a composition may be by various parenteral routes such as subcutaneous, intravenous, intradermal, intramuscular, intraperitoneal, intranasal, oral or transdermal routes. Parenteral administration can be accomplished by bolus injection or by gradual perfusion over time.

A typical regimen for preventing, suppressing, or treating an influenza virus related pathology, comprises administration of an effective amount of a vaccine composition as described herein, administered as a single treatment, or repeated as enhancing or booster dosages, over a period up to and including between one week and about 24 months, or any range or value therein.

According to the present disclosure, an “effective amount” of a composition is one that is sufficient to achieve a desired effect. It is understood that the effective dosage may be dependent upon the species, age, sex, health, and weight of the recipient, kind of concurrent treatment, if any, frequency of treatment, and the nature of the effect wanted. The ranges of effective doses provided below are not intended to limit the disclosure and represent dose ranges.

The dosage of a live, attenuated or killed virus vaccine for an animal such as a mammalian adult organism may be from about 10²-10²⁰, e.g., 10³-10¹², 10²-10¹⁰, 10⁵-10¹¹, 10⁶-10¹⁵, 10²-10¹⁰, or 10¹-10²⁰ plaque forming units (PFU)/kg, or any range or value therein. The dose of one viral isolate vaccine, e.g., in an inactivated vaccine, may range from about 0.1 to 1000, e.g., 0.1 to 10 μg, 1 to 20 μg, 30 to 100 μg, 10 to 50 μg, 50 to 200 μg, or 150 to 300 μg, of HA protein. However, the dosage should be a safe and effective amount as determined by conventional methods, using existing vaccines as a starting point.

The dosage of immunoreactive HA in each dose of replicated virus vaccine may be standardized to contain a suitable amount, e.g., 30 to 100 μg or any range or value therein, or the amount recommended by government agencies or recognized professional organizations. The quantity of NA can also be standardized, however, this glycoprotein may be labile during purification and storage.

The dosage of immunoreactive HA in each dose of replicated virus vaccine can be standardized to contain a suitable amount, e.g., 1-50 μg or any range or value therein, or the amount recommended by the U.S. Public Heath Service (PHS), which is usually 15 μg, per component for older children >3 years of age, and 7.5 μg per component for children <3 years of age. The quantity of NA can also be standardized, however, this glycoprotein can be labile during the processor purification and storage (Kendal et al., 1980. Kerr et al., 1975). Each 0.5-ml dose of vaccine may contain approximately 0.1 to 0.5 billion viral particles, 0.5 to 2 billion viral particles, 1 to 50 billion virus particles, 1 to 10 billion viral particles, 20 to 40 billion viral particles, 1 to 5 billion viral particles, or 40 to 80 billion viral particles.

The invention will be further described by the following non-limiting example.

### EXAMPLE

Broadly protective vaccines. Efforts are underway to develop broadly protective vaccines against IAVs and IBVs. Antibodies recognizing the highly conserved stem region of HA are broadly reactive against several IAV subtypes, or even against IAVs and IBVs, but the stem epitopes are immunosubdominant. For IAVs, several strategies are being tested to focus immune responses towards these immunosubdominant epitopes in the HA stem, including sequential immunizations with recombinant viruses possessing HA heads from different subtypes (to avoid immune-focusing on previously encountered HA head sequences). This strategy has also been tested for IBVs. To focus immune responses towards the IBV HA stem region, mice were sequentially immunized with viruses encoding the IBV HA stem region, but the H5, H7, and H8 IAV head regions. Vaccinated mice were protected against lethal challenge with several IBVs, but it is not known whether similar immune responses elicited by the immunosubdominant stem region would be protective in humans. Moreover, the need for sequential vaccinations would be a logistical challenge and increase costs. In a different approach, a peptide spanning the highly conserved IBV HA cleavage site was used to vaccine mice, resulting in protection against IBV challenges; since this epitope is immunosubdominant, it is unclear whether sufficient levels of protective antibodies can be elicited in humans.

### Methods to Prepare Broadly Protective Influenza B Viruses

The strategy to prepare broadly protective influenza B viruses included parental (representative) Victoria- and Yamagata-lineage viruses, e.g., see sequences below, introducing mutations into HA of the parental viruses at amino acid positions at which the two viruses differ and selecting mutants that are antigenically in-between the two lineages so that broadly reactive antibodies elicited by these antigens provide protection against viruses of both lineages. Due to their increased range of protection, the present vaccines will also provide benefits when there is an antigenic mismatch between the selected vaccine virus and the circulating strains.

Phuket mature HA sequence
(SEQ ID NO: 7)
DRICTGITSSNSPHVVKTATQGEVNVTGVIPLTTT
PTKSYFANLKGTRTRGKLCPDCLNCTDLDVALGRP
MCVGTTPSAKASILHEVRPVTSGCFPIMHDRTKIR
QLPNLLRGYEKIRLSTQNVIDAEKAPGGPYRLGTS
GSCPNATSKIGFFATMAWAVPKDNYKNATNPLTVE
VPYICTEGEDQITVWGFHSDNKTQMKSLYGDSNPQ
KFTSSANGVTTHYVSQIGDFPDQTEDGGLPQSGRI
VVDYMMQKPGKTGTIVYQRGVLLPQKVWCASGRSK
VIKGSLPLIGEADCLHEEYGGLNKSKPYYTGKHAK
AIGNCPIWVKTPLKLANGTKYRPPAKLLKERGFFG
AIAGFLEGGWEGMIAGWHGYTSHGAHGVAVAADLK
STQEAINKITKNLNSLSELEVKNLQRLSGAMDELH
NEILELDEKVDDLRADTISSQIELAVLLSNEGUIN
SEDEHLLALERKLKKMLGPSAVDIGNGCFETKHKC
NQTCLDRIAAGTFNAGEFSLPTFDSLNITAASLND
DGLDNHTILLYYSTAASSLAVTLMLAIFIVYMVSR
DNVSCSICL
Washington mature HA sequence
(SEQ ID NO: 8)
DRICTGITSSNSPHVVKTATQGEVNVTGVIPLTTT
PTKSHFANLKGTETRGKLCPKCLNCTDLDVALGRP
KCTGKIPSARVSILHEVRPVTSGCFPIMHDRTKIR
QLPNLLRGYEHVRLSTHNVINAEDAPGRPYEIGTS
GSCPNITNGNGFFATMAWAVPKNKTATNPLTIEVP
YICTEGEDQITVWGFHSDNETQMAKLYGDSKPQKF
TSSANGVTTHYVSQIGGFPNQTEDGGLPQSGRIVV
DYMVQKSGKTGTITYQRGILLPQKVWCASGRSKVI
KGSLPLIGEADCLHEKYGGLNKSKPYYTGEHAKAI
GNCPIWVKTPLKLANGTKYRPPAKLLKERGFFGAI
AGFLEGGWEGMIAGWHGYTSHGAHGVAVAADLKST
QEAINKITKNLNSLSELEVKNLQRLSGAMDELHNE
ILELDEKVDDLRADTISSQIELAVLLSNEGIINSE
DEHLLALERKLKKMLGPSAVEIGNGCFETKHKCNQ
TCLDRIAAGTFDAGEFSLPTFDSLNITAASLNDDG
LDNHTILLYYSTAASSLAVTLMIAIFVVYMVSRDN
VSCSICL

Alignment of Phuket (upper; SEQ ID NO:7) and Washington (lower; * indicates deletion relative to Phuket; SEQ ID NO:8) sequences. Exemplary residues to alter are in red font.

DRICTGITSSNSPHVVKTATQGEVNVTGVIPLTTTPTKSYFANLK
DRICTGITSSNSPHVVKTATQGEVNVTGVIPLTTTPTKSHFANLK
GTRTRGKLCPDCLNCTDLDVALGRPMCVGTTPSAKASILHEVRP
GTETRGKLCPKCLNCTDLDVALGRPKCTGKTPSARVSILHEVRP
VTSGCFPIMHDRTKIRQLPNLLRGYEKIRLSTQNVIDAEKAPGGP
VTSGCFPIMHDRTKIRQLPNLLRGYEKIRLSTQNVIDAEKAPGGP
YRLGTSGSCPNATSKIGFFATMAWAVPKDNYKNATNPLTVEVP
YEIGTSGSCPNATNGNGFFATMAWAVP**KNKTATNPLTIEVP
YICTEGEDQITVWGFHSDNKTQMKSLYGDSNPQKFTSSANGVT
YICTEGEDQITVWGFHSDNETQMAKLYGDSKPQKFTSSANGVT
THYVSQIGDFPDQTEDGGLPQSGRIVVDYMMQKPGKTGTIVYQR
THYVSQIGGFPNQTEDGGLPQSGRIVVDYMVQKSGKTGTITYQR
GVLLPQKVWCASGRSKVIKGSLPLIGEADCLHEEYGGLNKSKPY
GILLPQKVWCASGRSKVIKGSLPLIGEADCLHEKYGGLNKSKPY
(SEQ ID NO: 9)
YTGKHAKAIGNC
(SEQ ID NO: 10)
YTGEHAKAIGNC

Adjuvants. Aluminum adjuvants have been used widely in human and veterinary vaccines due to their effectiveness, excellent safety profile, low reactogenicity, and low costs. Two major types of aluminum adjuvants (i.e., aluminum hydroxide and aluminum phosphate have been used in several human vaccines, including Anthrax, hepatitis A, hepatitis B, and human papillomavirus vaccines. Aluminum adjuvants adsorb the antigen and present it in a particulate form, resulting in increased uptake by antigen-presenting cells. The use of aluminum adjuvants increases Th2-mediated immune responses, but has little effect on Th1-mediated immune responses. Ferret and hamster studies showed that ‘Alhydrogel Adjuvant 2%’ (Alhydrogel, an aluminum hydroxide (Al(OH)₃) suspension) is superior to other adjuvants (e.g., Addavax and Quil-A) in stimulating antibody responses to IAV HA protein in ferrets and Syrian hamsters.

As disclosed herein, mutating amino acids that differ between representative Victoria- and Yamagata-lineage viruses, generates vaccine viruses that are antigenically in-between the two lineages, which may provide protection against both Victoria- and Yamagata-lineage viruses, e.g., based on testing their immunogenicity and protective efficacy against homologous and heterologous IBVs in ferrets, the preferred animal model for influenza vaccination and challenge studies. One might argue that ancestral IBVs (circulating before the two lineages separated) could be used as vaccines that provide protection against both IBV lineages. However, the present candidates are based on recently circulating viruses and bear amino acids in their potential antigenic epitopes that are encoded by recent IBV vaccine viruses.

### Data

High-yield backbone. Efficient vaccine virus replication in embryonated chicken eggs or cells certified for human vaccine production is vital for the large-scale production of inactivated or live attenuated influenza vaccines. Vaccine viruses often require several passages in their growth substrate (i.e., embryonated chicken eggs or cultured cells) to acquire adaptive mutations that increase replication rates. A high-yield influenza B virus backbone suitable for both IBV lineages was developed, e.g., HY (Yam) NP-P40S, M1-R77K, NS1-K176Q, NS-a39g, PA-a2272t and HY (Vic) NP-P40S/M204T, M1-M86T, NS-(38+1)g, PA-a2272t. The former was used to prepare recombinant viruses in the experiments disclosed herein.

Briefly, virus libraries possessing random mutations in the six “internal” influenza B viral RNA segments (i.e., those not encoding the major viral antigens, HA and NA) were screened for mutants that confer efficient replication. Candidate viruses that supported high yield in cell culture were tested with the HA and NA genes of eight different viruses of both IBV lineages. Combinations of mutations were identified that increased the titers of candidate vaccine viruses in mammalian cells used for human influenza vaccine virus propagation and in embryonated chicken eggs, the most common propagation system for influenza viruses. IBV HA mutants that elicit broadly protective immune responses may be presented from inactivated vaccines based on our high-yield backbone.

Antigenic cartography. Antigenic cartography (developed by D. Smith, Cambridge University, and Ron Fouchier, Erasmus Medical Center, Rotterdam) allows the visualization and interpretation of antigenic relationships of viruses and sera, based on data from hemagglutination inhibition (HI) assays or focus reduction assays (FRA). It is similar to geographic maps in that individual distance measurements (from points A to B, A to C, B to C, etc.) are converted into two-dimensional maps or three-dimensional representations that show the distances among the data points. In essence, the closer the antigenic similarity of two viruses or sera, the closer they will be in the map. The Smith and Fouchier groups showed that human H3N2 viruses form clusters of antigenically similar (although not identical) viruses, until mutations in antigenic epitopes lead to substantial changes in antigenic properties that create a new antigenic cluster and result in an update of the vaccine strains. Antigenic cartography has profoundly improved the interpretation of the antigenic relationships of IAVs of the H1N1, H3N2, and H5 subtypes, and is now employed by the WHO Strain Selection Committee when selecting new vaccine viruses. Antigenic cartography to analyze and predict the antigenic evolution of human H1N1, H3N2, and pandemic H5 IAVs. In contrast to IAV, comprehensive maps that span several decades of IBV antigenic evolution and include both IBV lineages are lacking (see, e.g., Rosu et al., Proc Natl Acad Sci USA. 2022 Oct. 18; 119(42):e2211616119, the disclosure of which is incorporated by reference herein). Such maps are useful to assess the antigenic positions of the present vaccinate candidates relative to both lineages.

A strategy to develop a broadly protective IBV vaccine. A goal is to develop a vaccine that protects against viruses of both IBV lineages. The antigenic differences between the two IBV lineages likely result from the amino acid differences between them and therefore we compared the amino acid sequences of the HA1 subunits (amino acids 40-313; numbering is based on a mature Victoria-lineage HA) of two recent IBVs, namely B/Florida/78/2015 (‘Florida/Vic’, a recent Victoria-lineage vaccine virus) and B/Phuket/3073/2013 (‘Phuket/Yam’, the current Yamagata-lineage vaccine virus). These two viruses differ by 38 amino acids in the region analyzed). Next, two synthetic gene libraries were designed based on the Florida/Vic and Phuket/Yam HA sequences that encode both of the parental amino acids at the respective amino acid position. For example, Florida/Vic encodes N at position 126, whereas Phuket/Yam encodes D; thus, the synthetic gene libraries encode both N and D at position 126. Because of the genetic code, it is not always possible to design codons encoding only the two amino acids of the parental viruses. For example, at position 129, Florida/Vic and Phuket/Yam encode D and K, respectively; however, there is no codon encoding only D and K. Therefore, the synthetic library encodes these two amino acids, but also N and E. As a result, two-to-four different amino acids are encoded at each of the 38 positions at which the HA proteins differ, yielding synthetic gene libraries that theoretically encode 2-4³⁸ amino acid combinations. The synthetic gene libraries were cloned into vectors encoding the remainders of the Florida/Vic or Phuket/Yam HAs, respectively.

A reverse genetics system was used to create virus libraries composed of the six internal genes of high-yield B/Yamagata/173, the NA vRNAs of Florida/Vic or Phuket/Yam, respectively, and the Florida/Vic or Phuket/Yam HA libraries, respectively. Forty-eight hours later, the virus libraries were collected from plasmid-transfected human embryonic kidney (293T) cells and inoculated onto Madin-Darby canine kidney (MDCK) cells for plaque assays. Mutations at up to 38 amino acid positions of HA may be expected to result in many non-functional mutants; however, at most positions, we started with only two amino acids that are known to be functional in the context of Florida/Vic or Phuket/Yam, respectively. Moreover, the strategy used here-reverse genetics followed by viral plaque assays-eliminates any non-viable mutants, which would not form virus plaques. Thus, while starting from a large number of theoretically possible (potentially non-viable) mutants, only viruses with a functional HA will be isolated from plaque assays.

After reverse genetics and viral plaque assays, >100 individual virus plaques based on the Florida/Vic or Phuket/Yam HAs, respectively, were isolated and sequenced. Numerous sequences were detected at the 38 mutated positions, with no particular sequence becoming dominant in the virus libraries. Next, the mutant viruses were tested in HI assays, in which two-fold serial dilutions of serum are mixed with a defined amount of virus and with turkey red blood cells. If the serum antibodies bind to the head region of HA, the virus can no longer agglutinate red blood cells (e.g., hemagglutination is inhibited). The HI titer is the reciprocal of the highest serum dilution at which hemagglutination occurs (higher HI titers indicate a stronger interaction between an antigen and an antibody). The mutant viruses were tested with ferret antisera to the parental Florida/Vic and Phuket/Yam virus. 19 mutants were identified for which the HI titers to the heterologous serum increased at least 4-fold, while the HI titers to the homologous serum were not reduced by more than 4-fold. An example is Florida/Vic Mutant #4, whose HI titer to Florida/Vic ferret antiserum remained high (HI titer of 5120), while this mutant gained reactivity to Phuket/Yam ferret antiserum: Wild-type Florida/Vic virus has a low HI titer of 20 against ferret serum to Phuket/Yam, a low HI titer of 20 against ferret serum to Phuket/Yam, whereas Florida/Vic Mutant #4 has an HI titer of 320 against ferret serum to Phuket/Yam. Sequence analysis of the 19 mutants did not reveal any obvious correlation between the amino acid sequences and the HI titers.

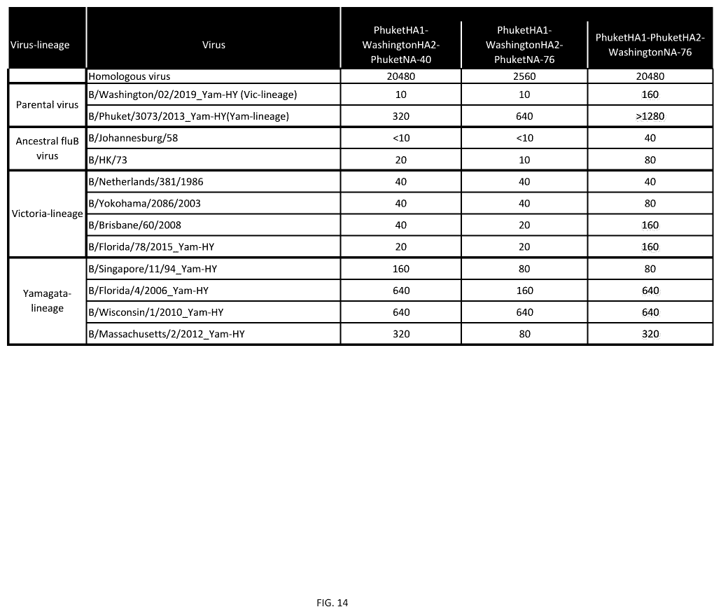

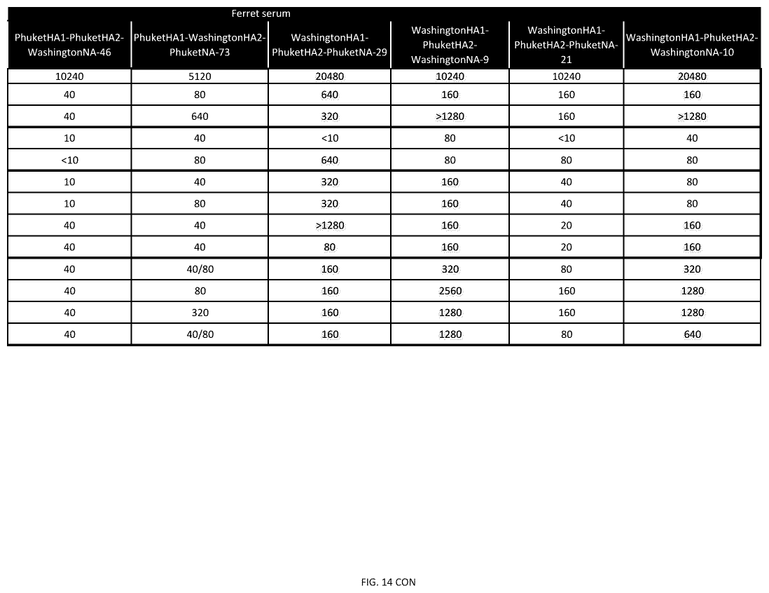

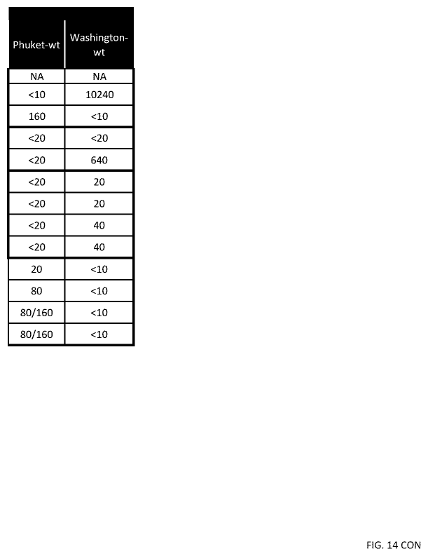

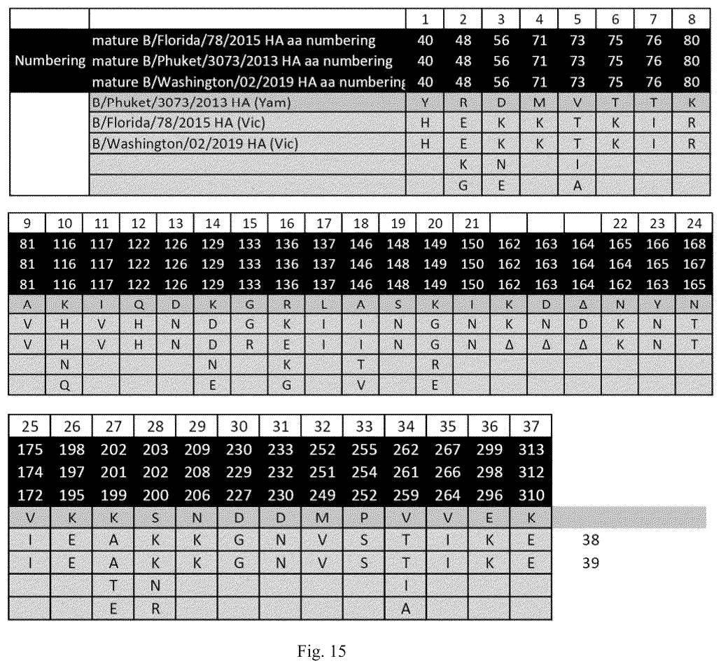

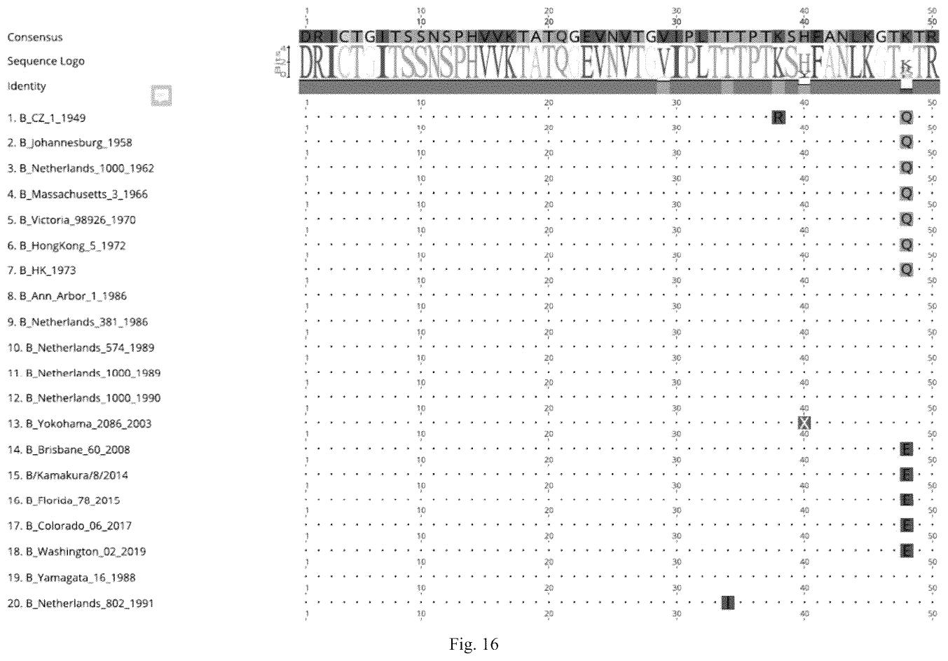

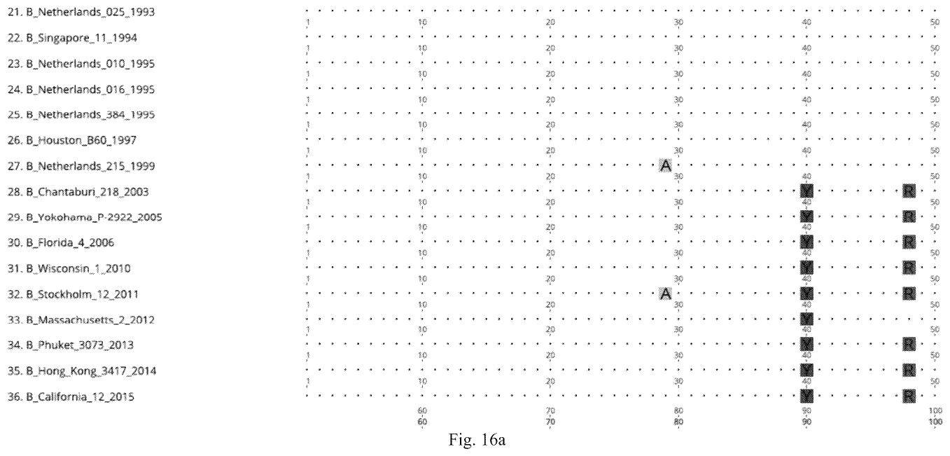

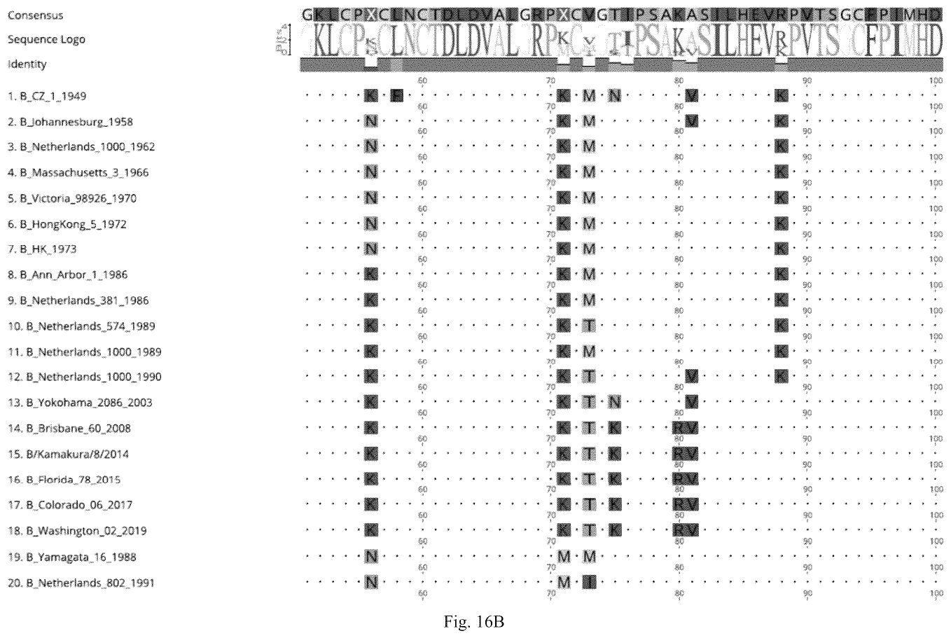

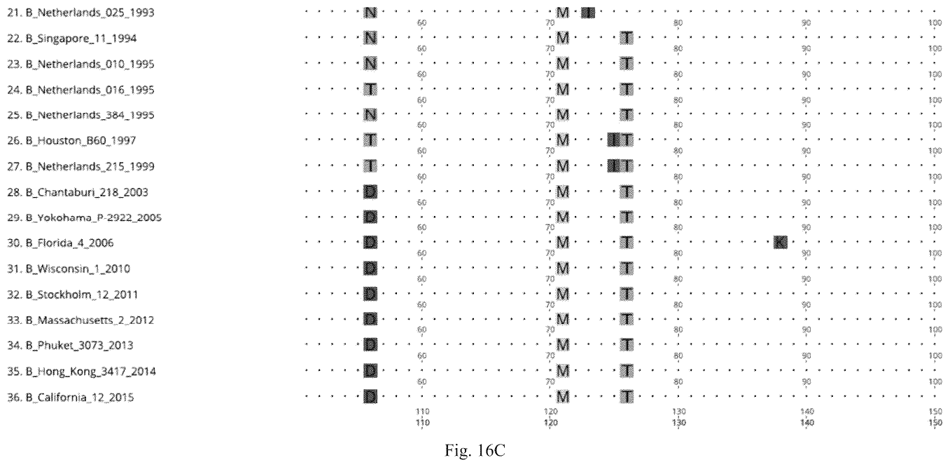

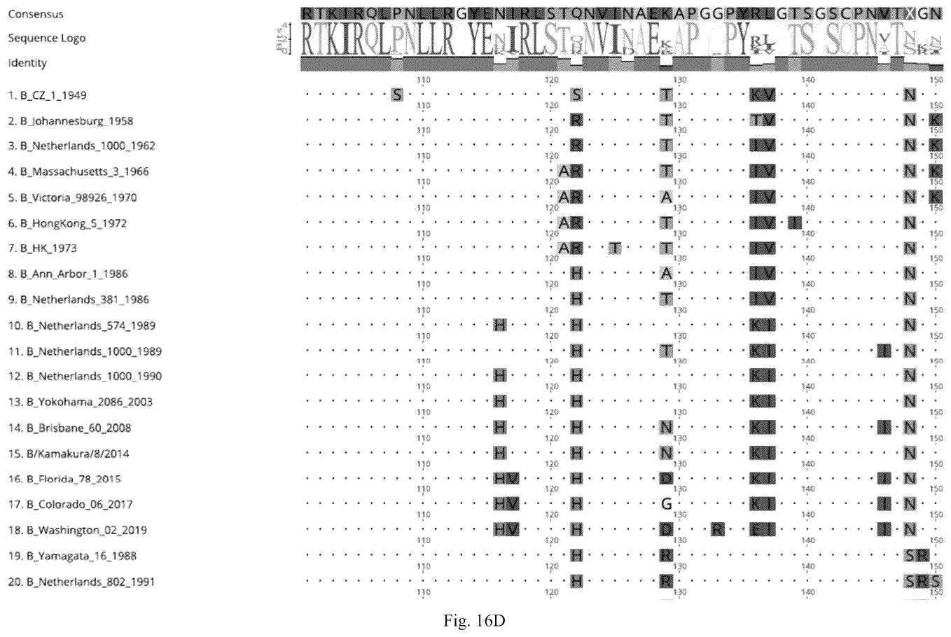

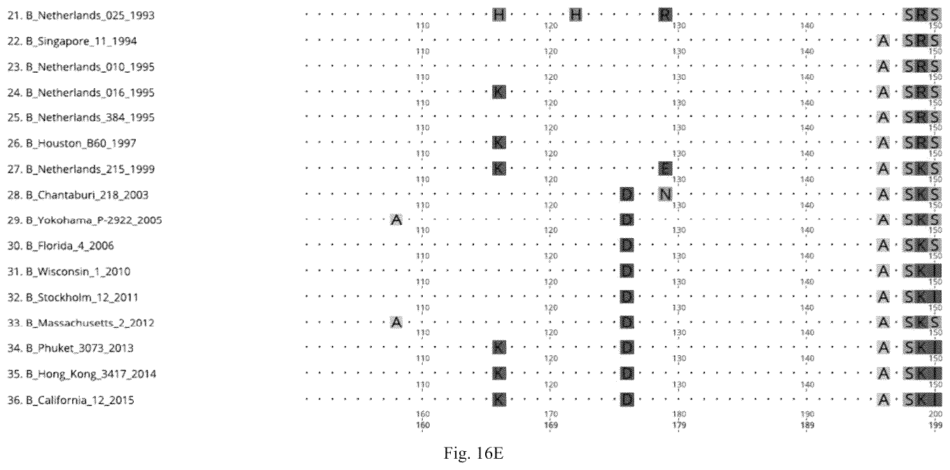

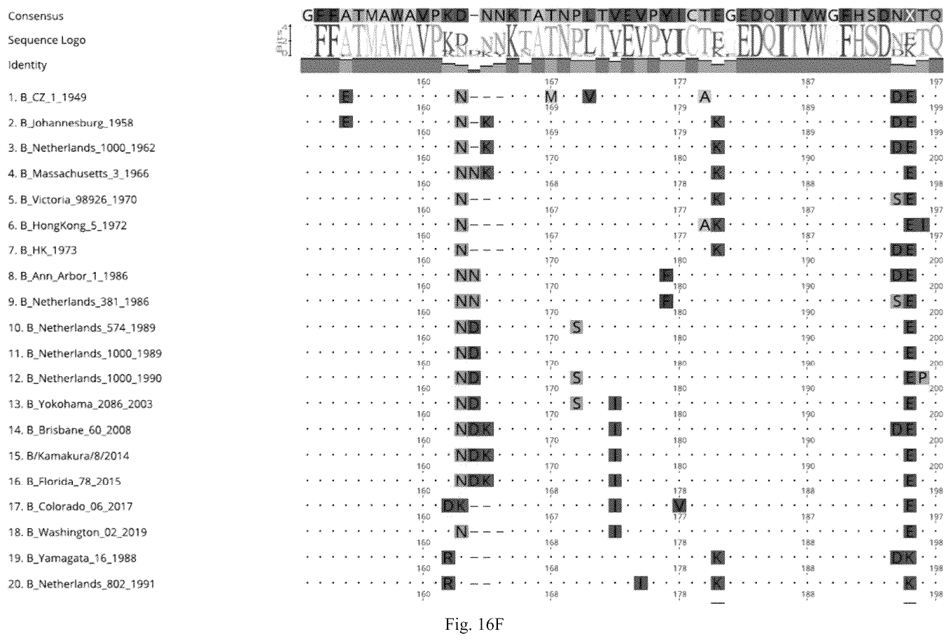

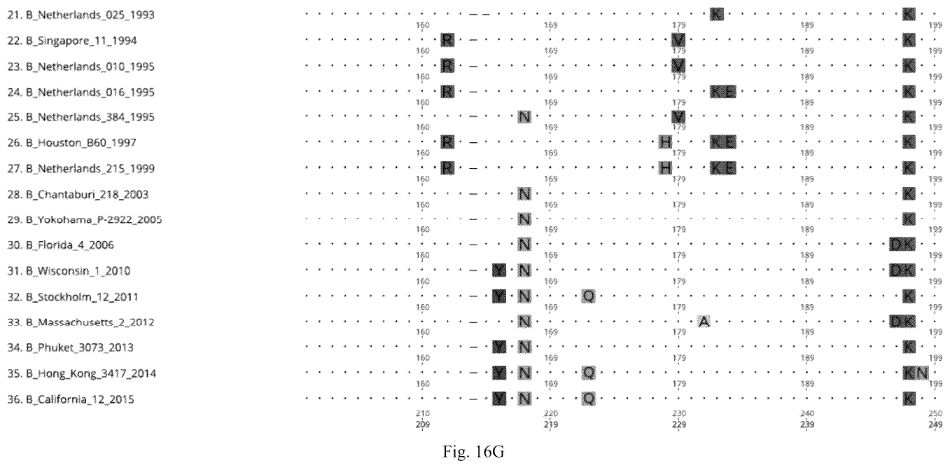

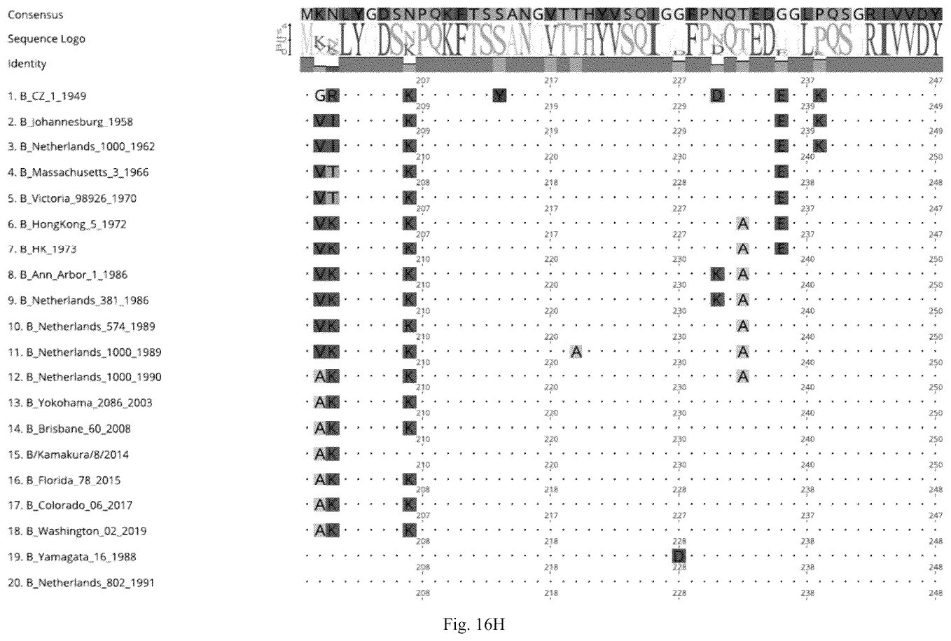

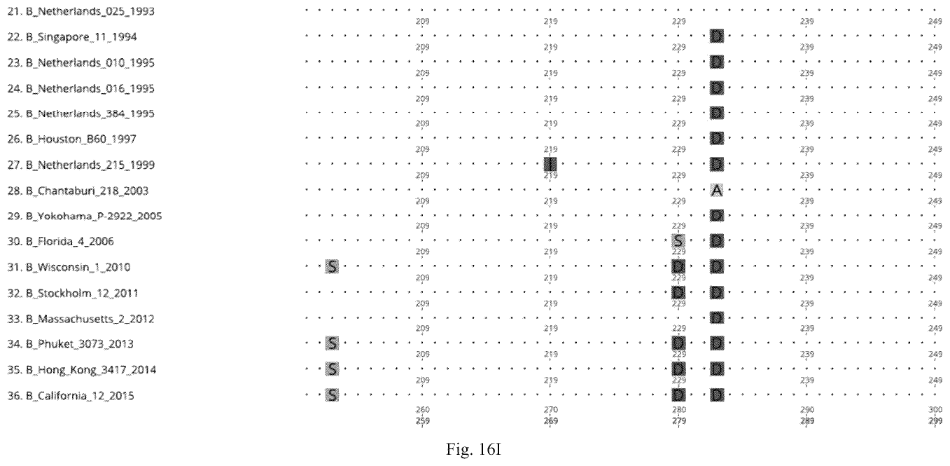

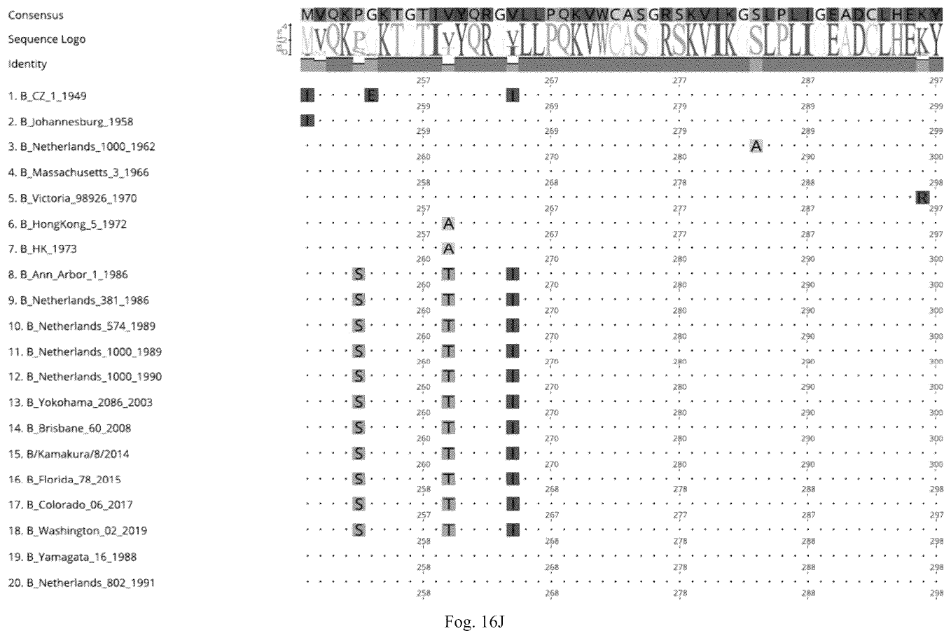

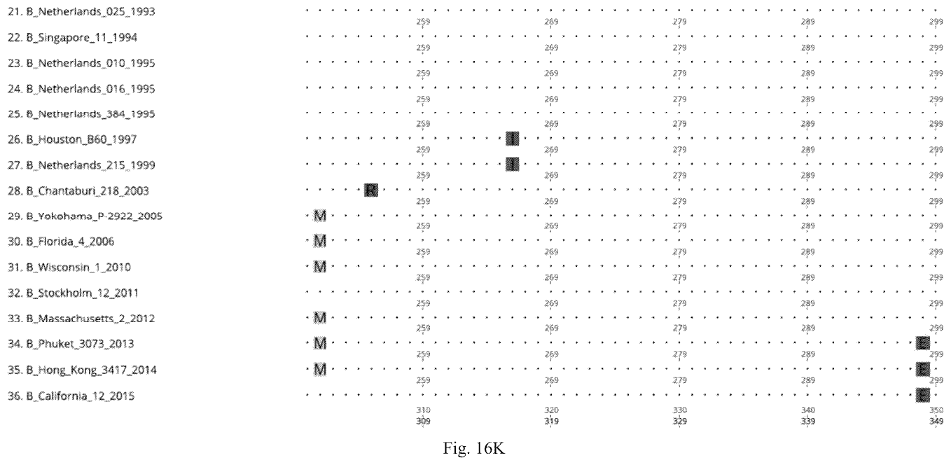

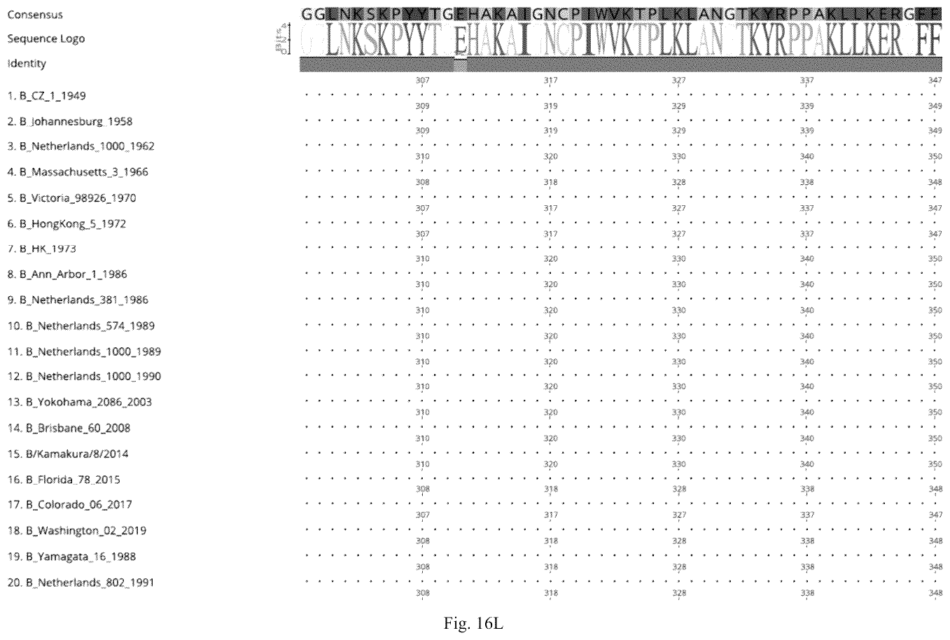

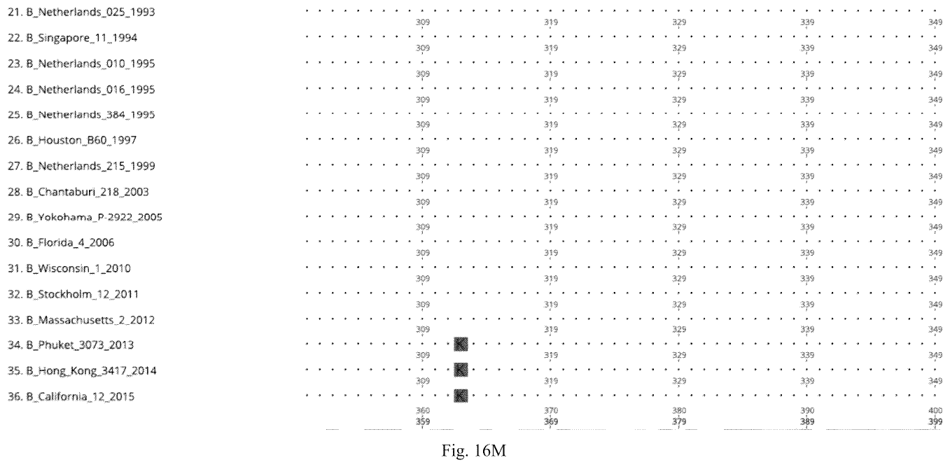

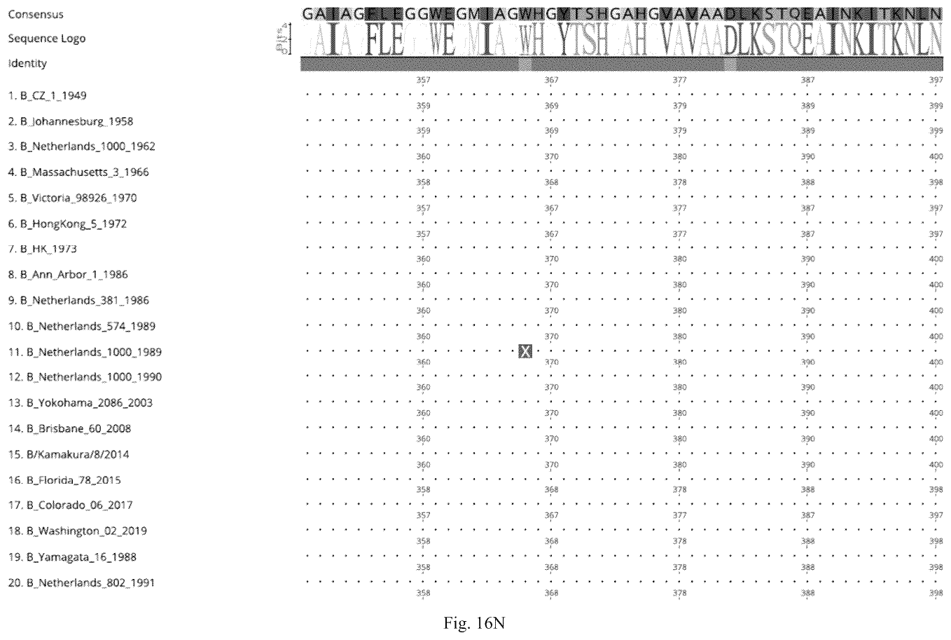

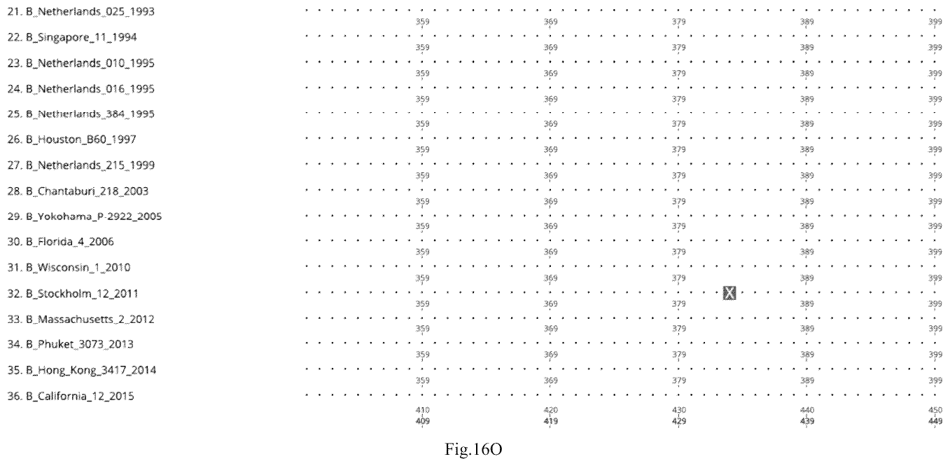

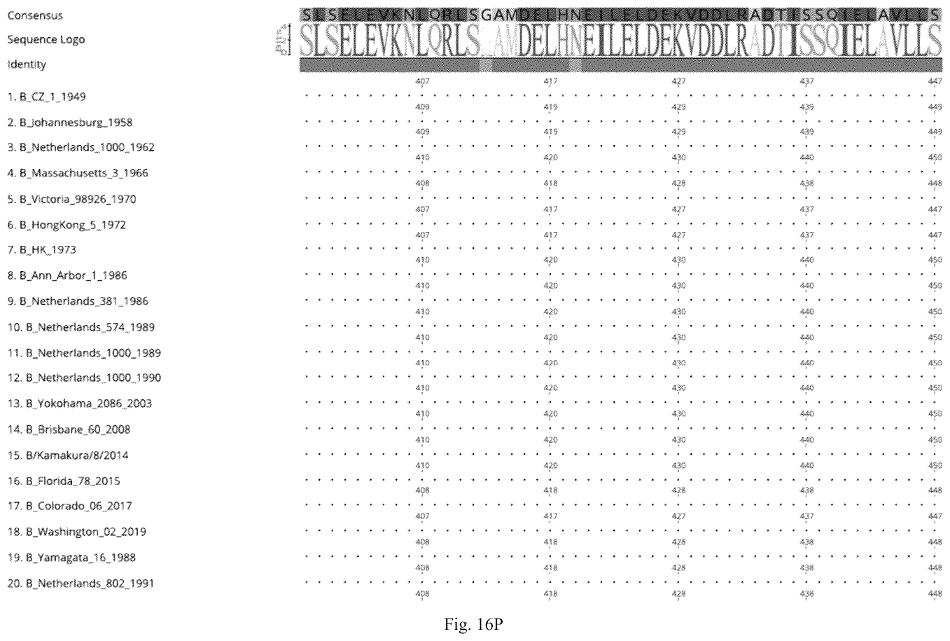

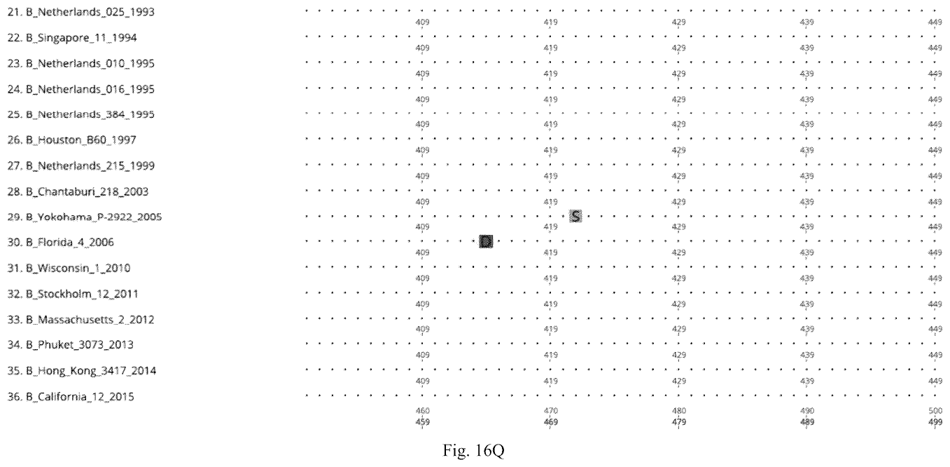

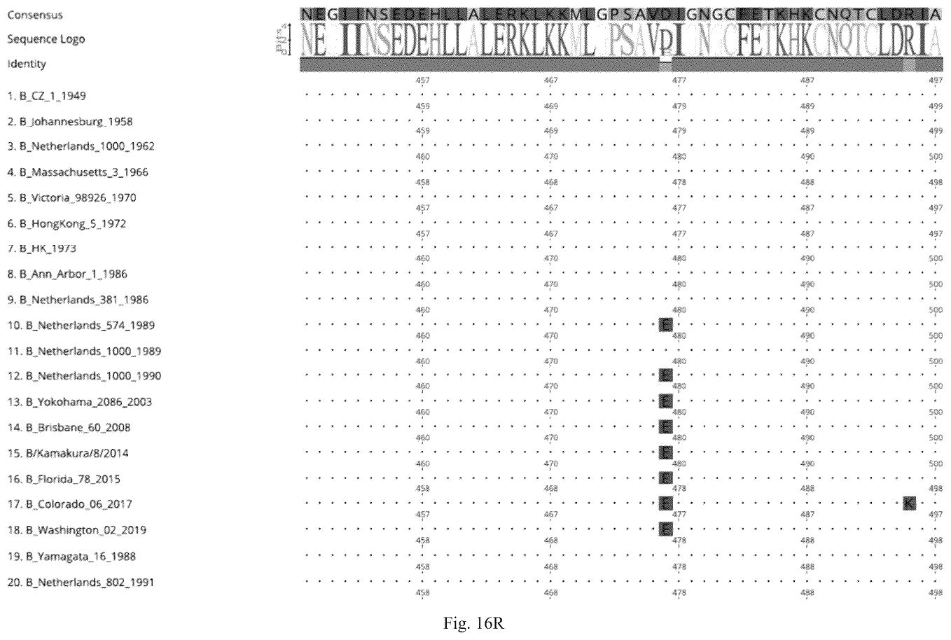

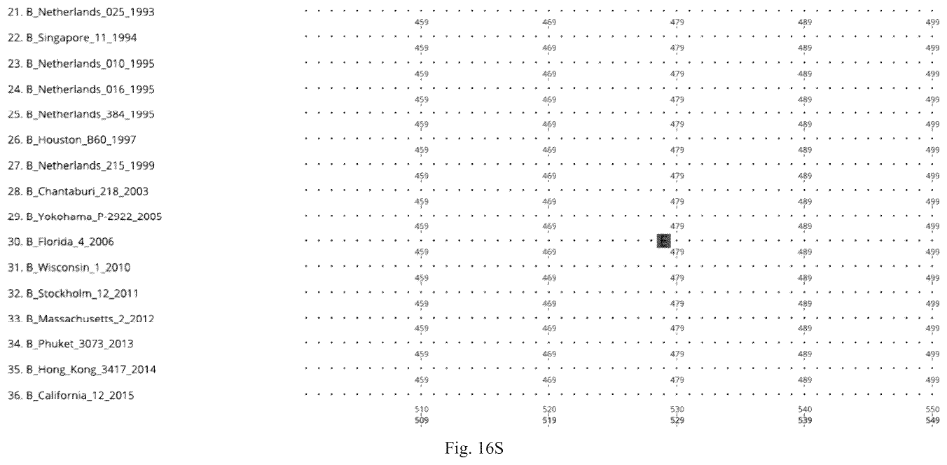

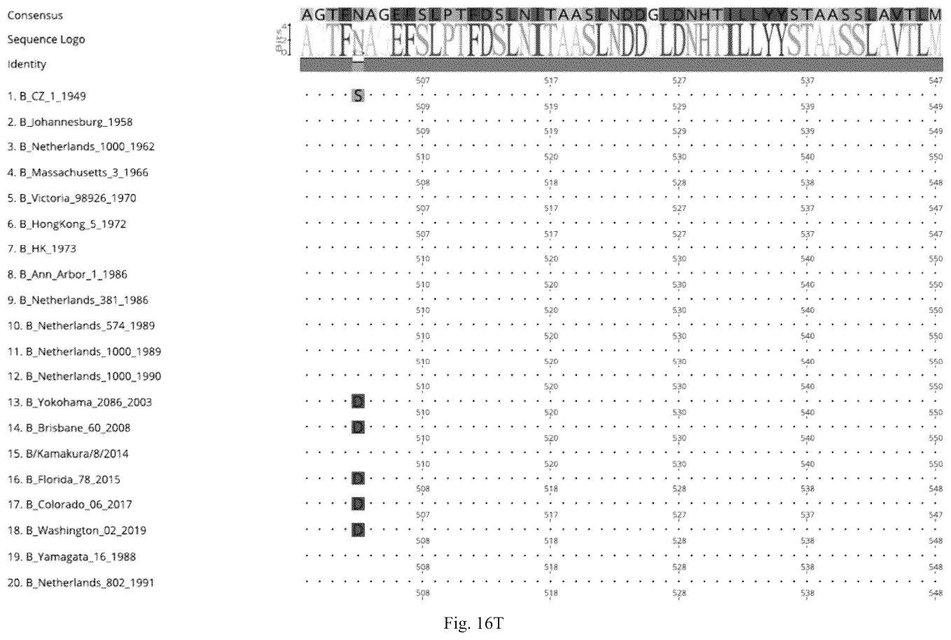

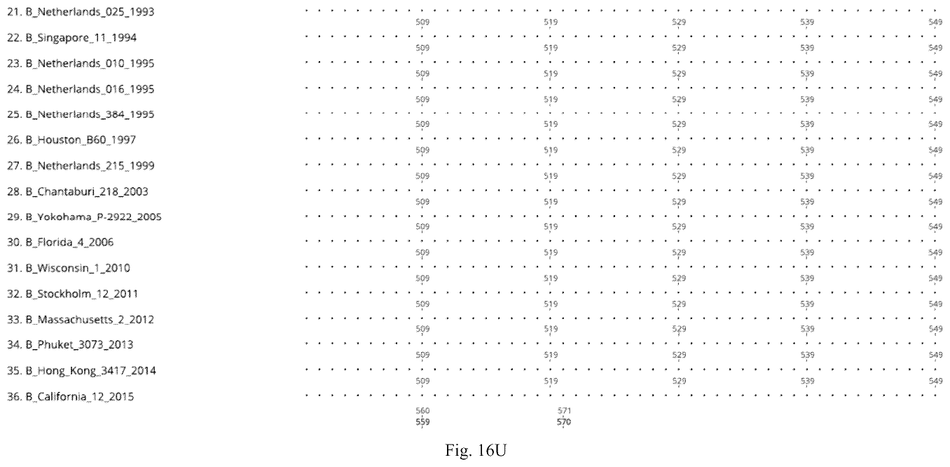

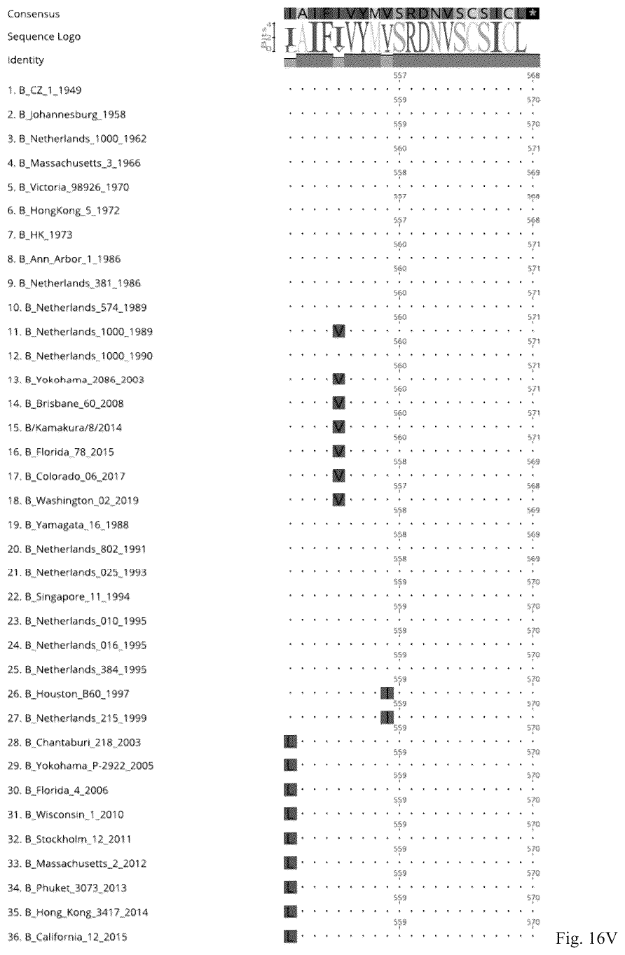

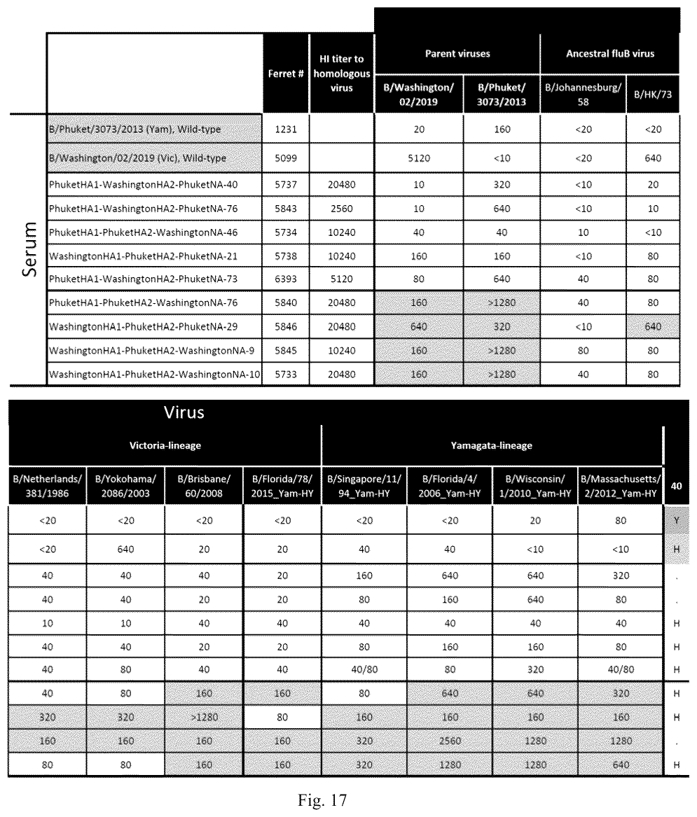

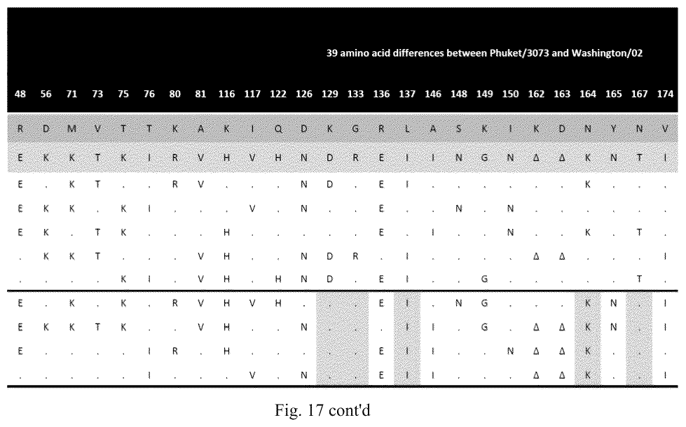

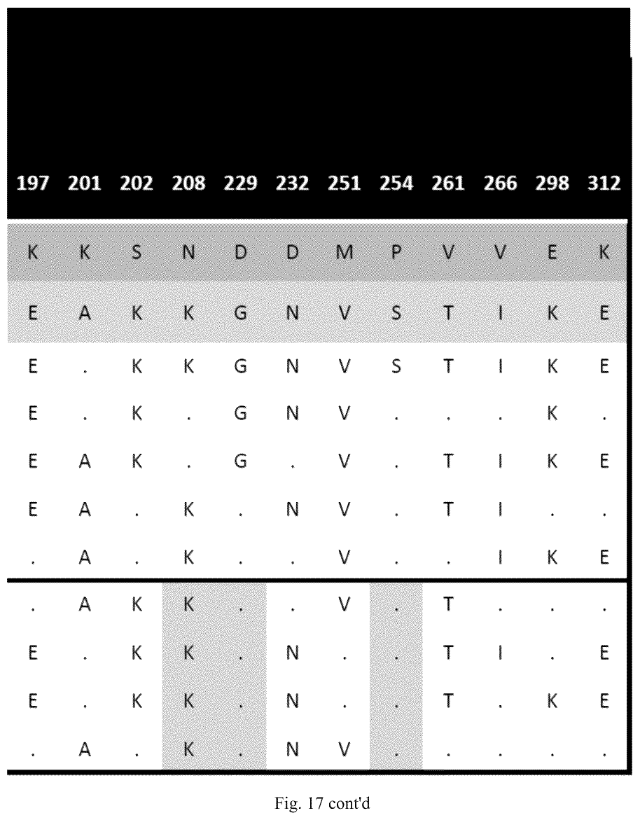

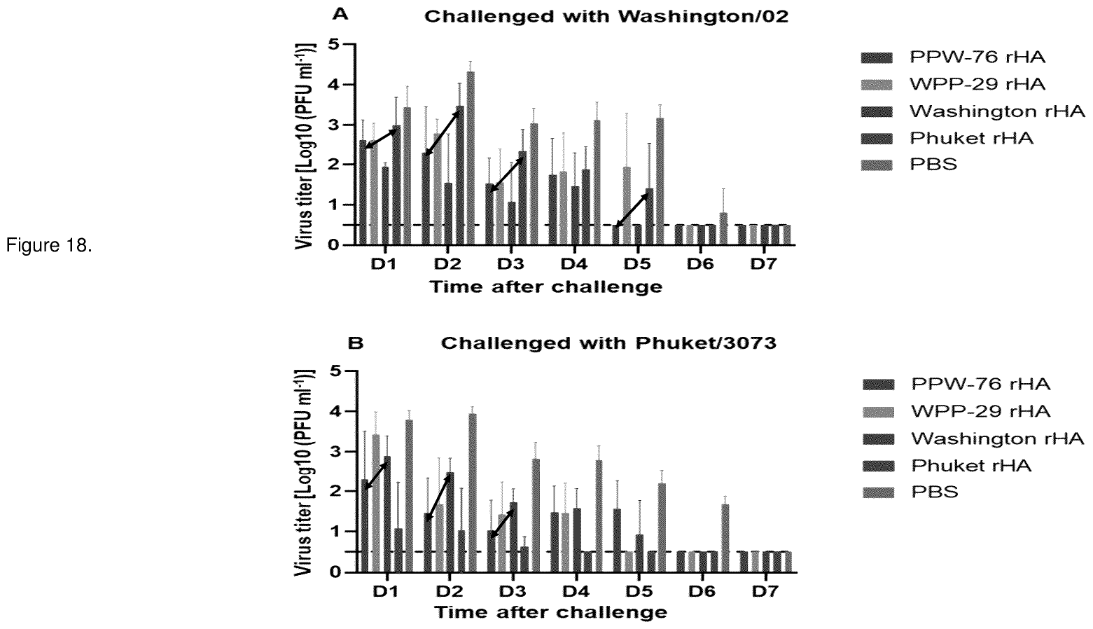

Virus libraries were generated based on the B/Phuket/3073/2013 and B/Washington/02/2019 HA proteins, which differ by 39 amino acids in the region targeted for mutagenesis. These libraries encode only the parental amino acids; i.e., in contrast to the B/Phuket/3073/2013-B/Florida/78/2015 libraries, no additional amino acids are encoded. Virus libraries were generated encoding B/Phuket/3073/2013-like amino acids in the context of B/Washington/02/2019 HA, and vice versa. For individual viruses isolated from plaque assays, the HI titers against sera to Phuket and Washington viruses were determined and the HA genes were sequenced. Selected mutants were further characterized in HI assays with a panel of ferret sera to various Yamagata- and Victoria-lineage viruses. Moreover, selected mutants were used to immunize ferrets, and the ferret sera were tested in HI assays to a panel of Yamagata- and Victoria-lineage viruses. We have now identified four mutants that stimulate highly cross-reactive ferret sera. The reactivity of the ferret sera is shown in FIG. 14 and FIG. 17. FIG. 17 also lists the amino acid sequences of the respective viruses at the targeted amino acid positions. The mutant viruses that elicit cross-protective ferret sera have amino acids in common at eight amino acid positions (highlighted in yellow in FIG. 17). These residues may be important for the broad reactivity of the sera elicited by these mutants.

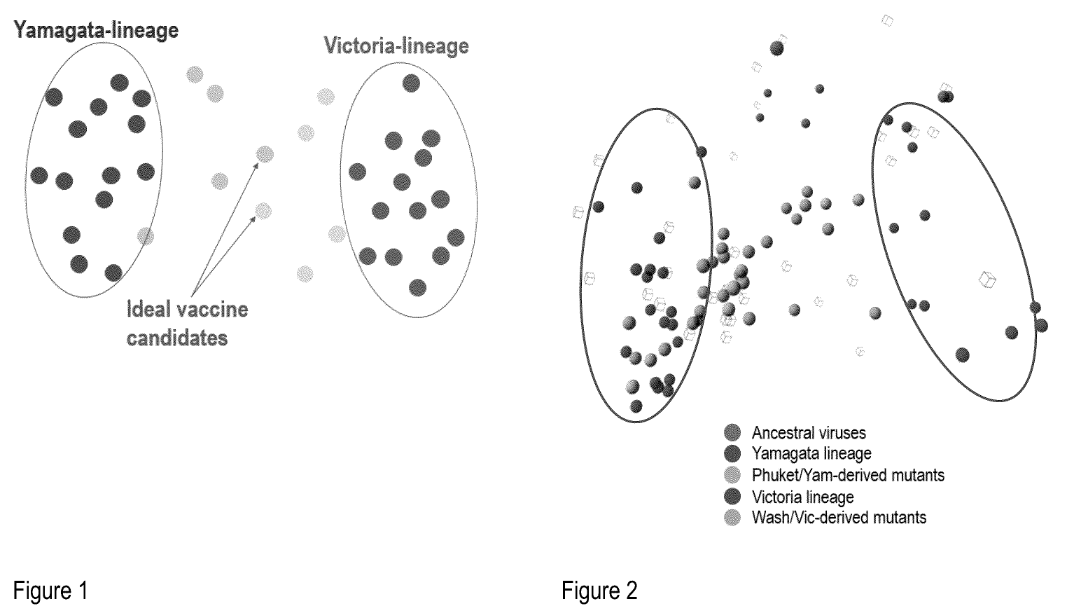

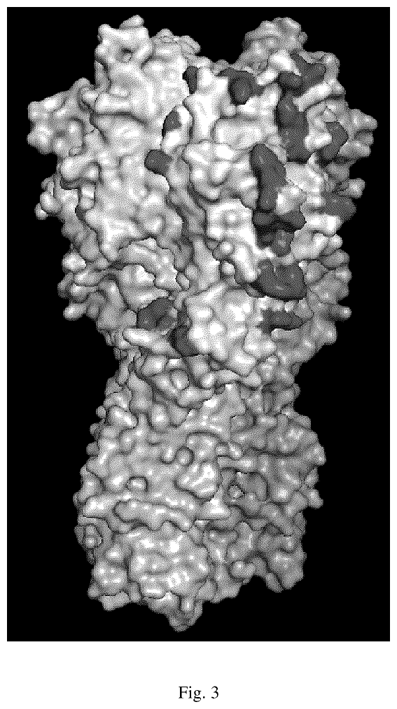

FIG. 1 shows a hypothetical antigenic map of IBVs and IBV mutants with viruses of the Yamagata- and Victoria-lineages shown in dark blue and brown, respectively. Hypothetical mutants are shown in light blue and gold, respectively. A vaccine candidate would be located in-between the two lineages. In contrast to the hypothetical map in FIG. 1, actual antigenic maps show both the location of the viruses (shown in circles in FIG. 2) and of the sera (shown in squares in FIG. 2).

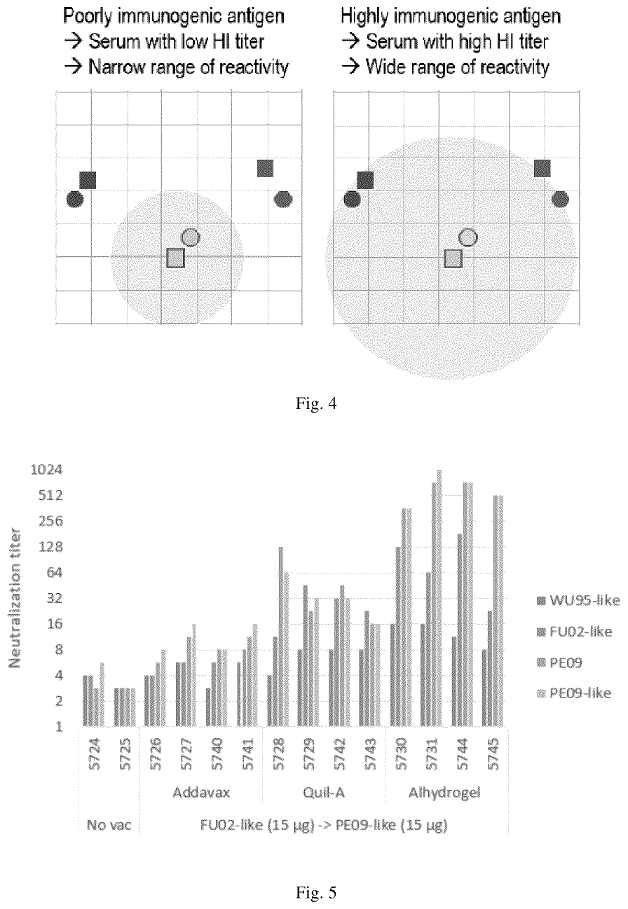

A poorly immunogenic antigen elicits serum with a low HI titer against the homologous antigen: this serum will have a limited ‘range’, it only reacts with antigens within a close range on the antigenic map (FIG. 4, left panel). However, highly immunogenic antigens elicit sera with high antibody titers to the homologous virus; such a serum is expected to react with a greater range of antigens (FIG. 4, right panel). A poorly immunogenic antigen as shown in the left panel would not provide protection against viruses of the two IBV lineages (shown in dark blue and brown in FIG. 4). This approach, which aims to bridge the antigenic differences between the two IBV lineages, may use a highly immunogenic antigen as shown in the right panel. If so, immune-stimulatory approaches including adjuvants may be employed

Immune-stimulatory effect of Alhydrogel. Recently, several adjuvants were tested for their immune-stimulatory effect in animals vaccinated with recombinant IAV HA protein. In one experiment, ferrets were sequentially vaccinated with recombinant HA representing the Fujian 2002 (FU02) and Perth 2009 (PE09) antigenic clusters, respectively. The recombinant HAs were adjuvanted with Addavax (a MF59-like oil-in water emulsion), Quil-A (a saponin), or Alhydrogel. Three weeks after the second immunization, sera were collected to assess the virus neutralization titers against viruses from the Wuhan 1995 (WU95; a cluster that circulated before the FU02 and PE09 clusters), FU02, or PE09 antigenic clusters. Alhydrogel elicited much higher neutralizing serum antibody titers than the other two adjuvants tested (FIG. 5). Similar data was obtained with Syrian hamsters immunized with adjuvanted IAV.

### To Develop Broadly Reactive Influenza B Candidate Vaccine Viruses

The goal is to develop broadly protective vaccines to IBVs. Preliminary studies identified IBV HA mutants that reacted with ferret sera raised against the other IBV lineage.

Generation of an antigenic map for IBV HAs. One key to building robust and high-quality antigenic maps is having antigens and antisera that are spread across the particular region of interest. Antigens are better coordinated in antigenic space when the antisera (used to measure the antigenic properties of the antigens) are spread out in the space of interest. As a rule of thumb, this is achieved with genetically diverse antigens. However, the location of the antigens and antisera cannot be predicted from their sequences. This means that antigenic cartography is usually an iterative process whereby a first antigenic map is generated and assessed for its robustness, followed by generating and testing additional antigens and antisera to fill potential ‘holes’ in the map.

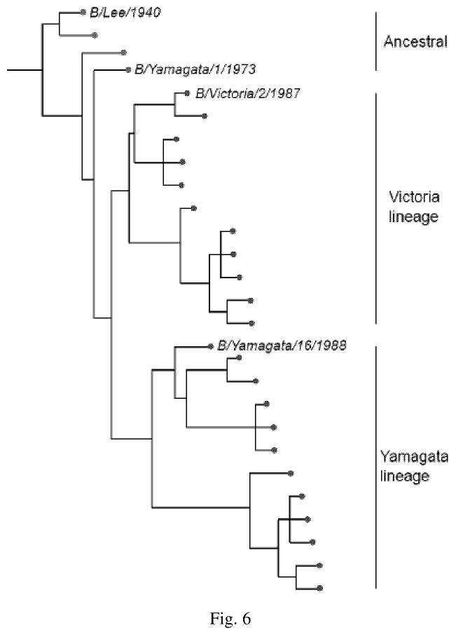

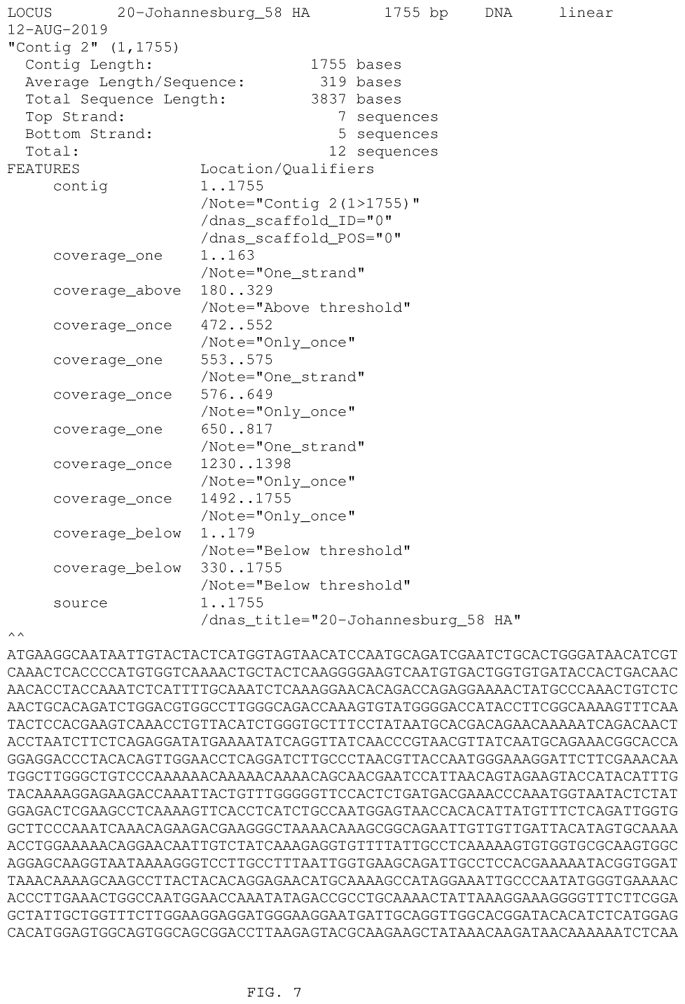

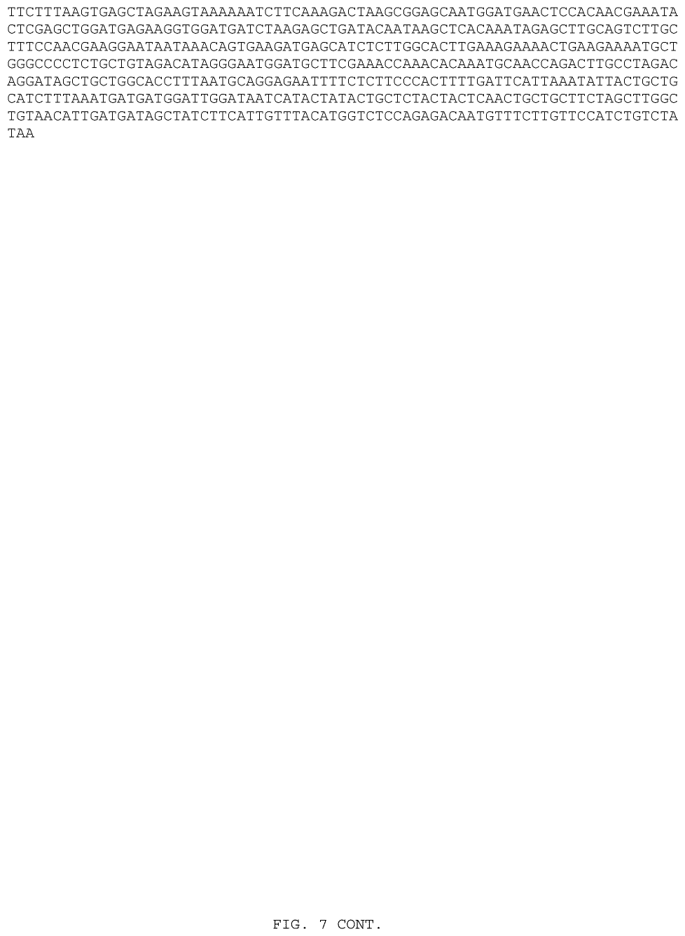

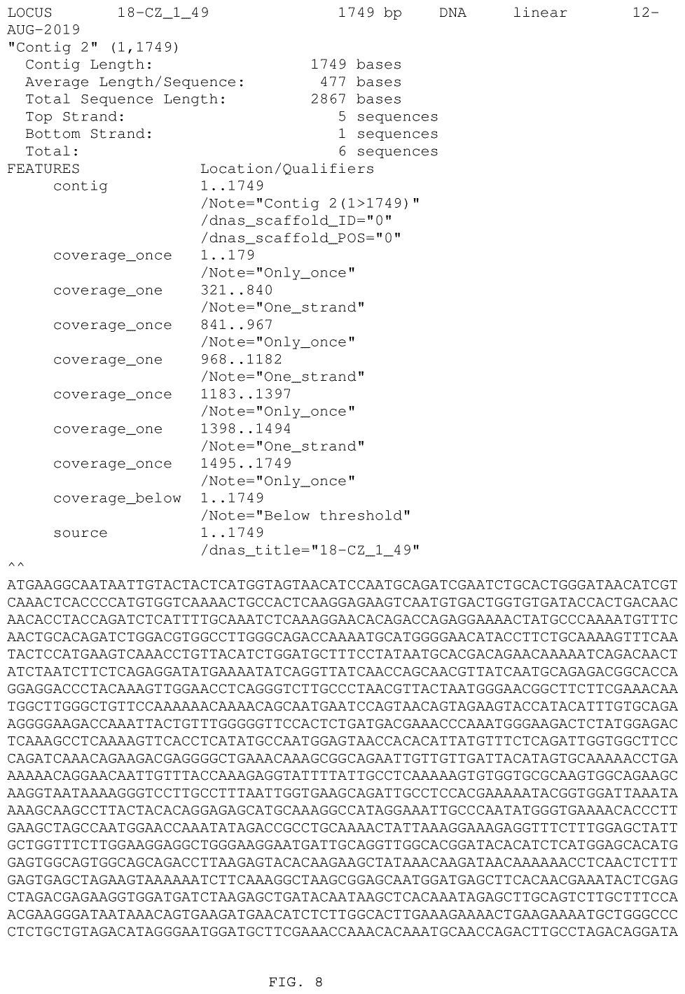

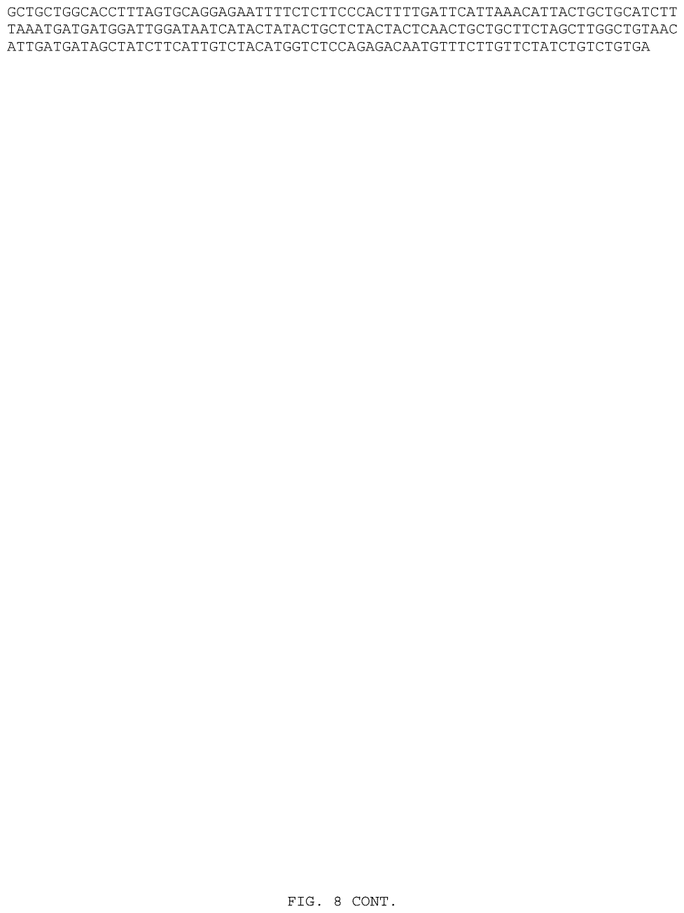

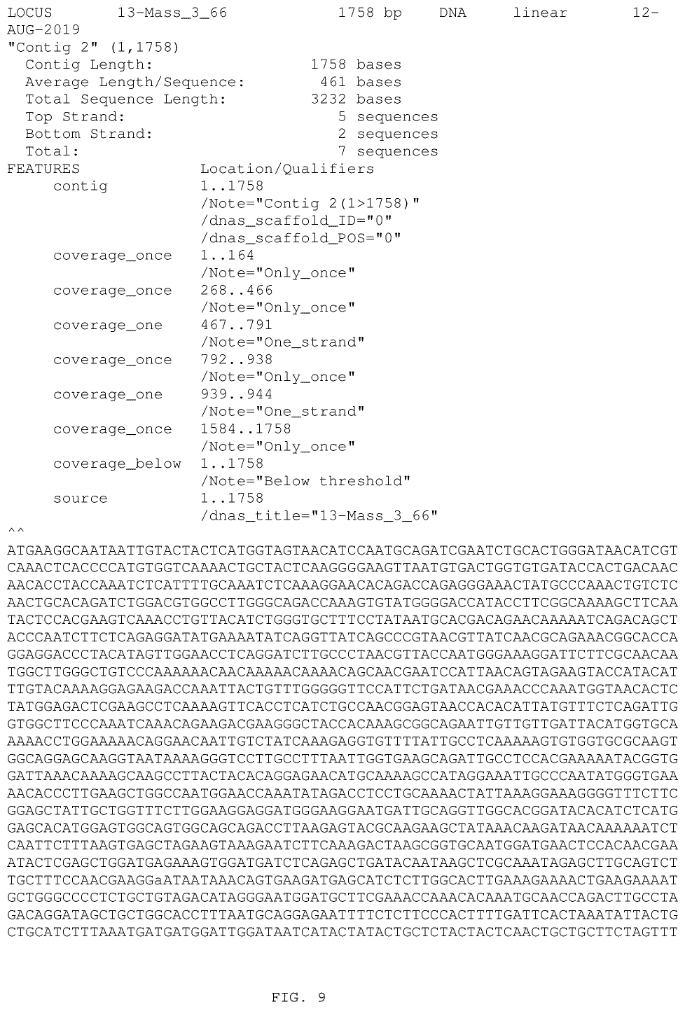

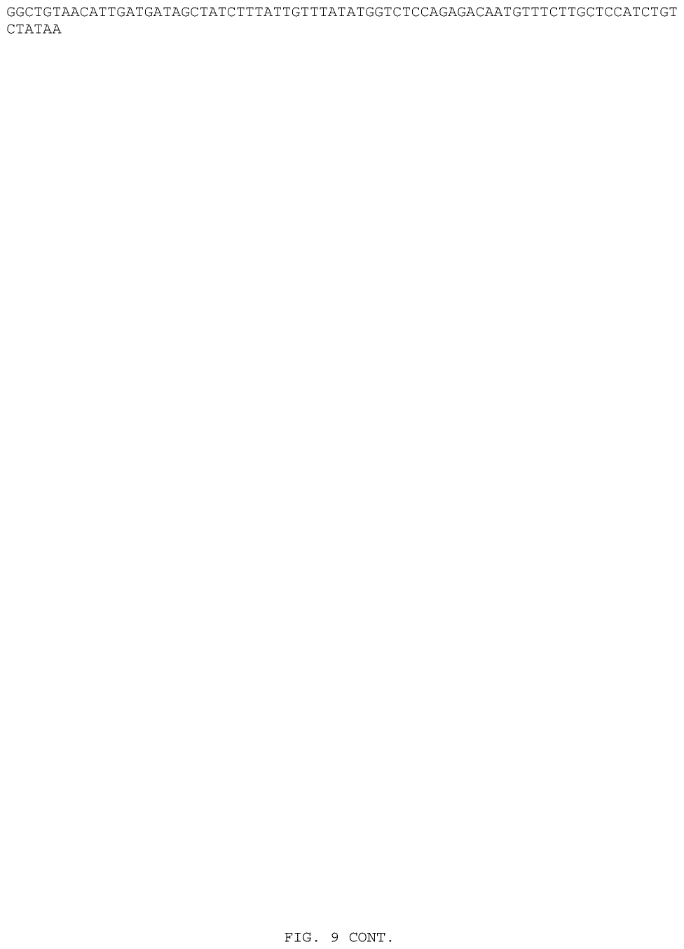

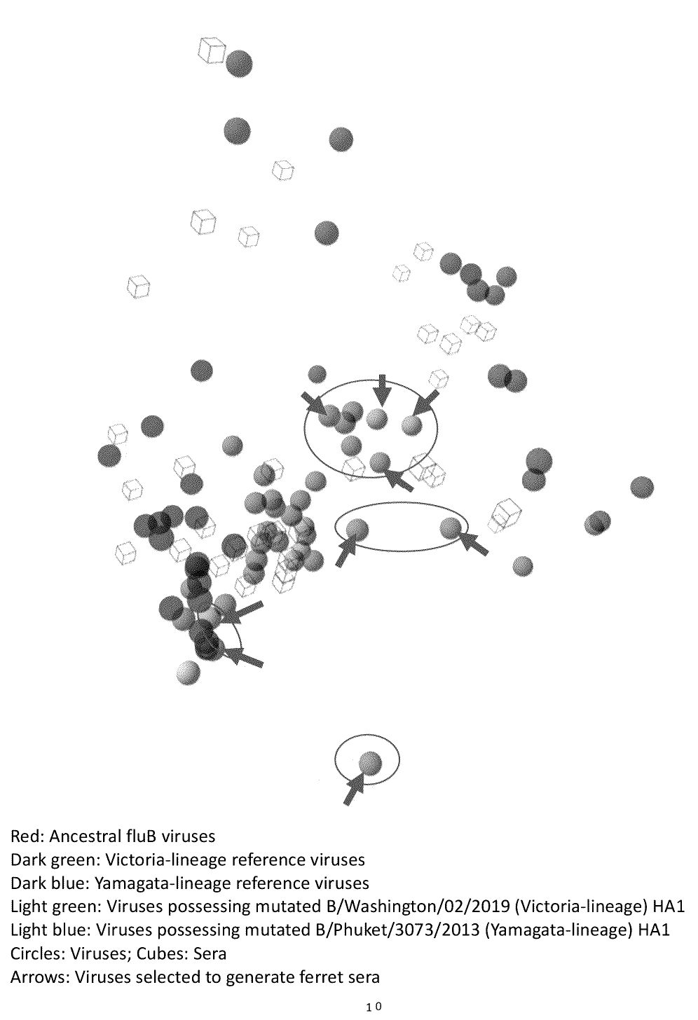

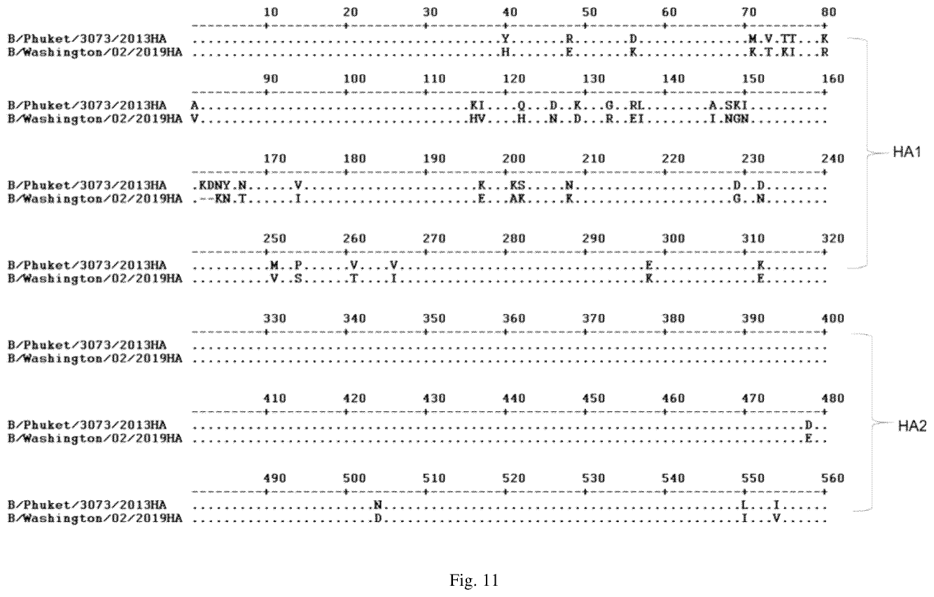

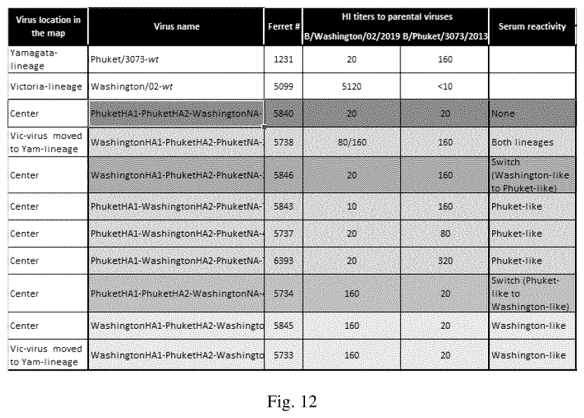

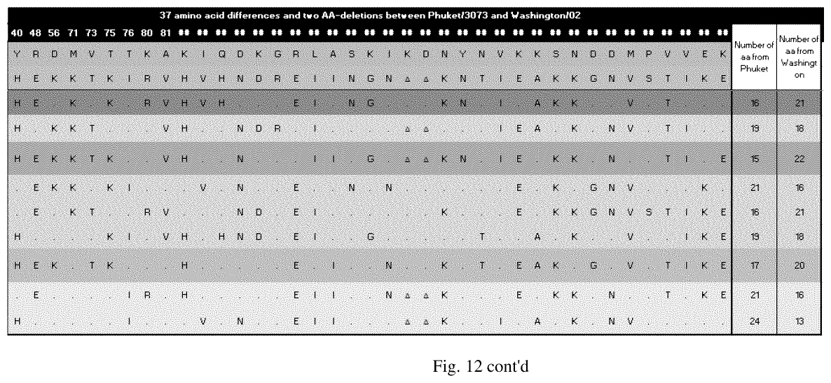

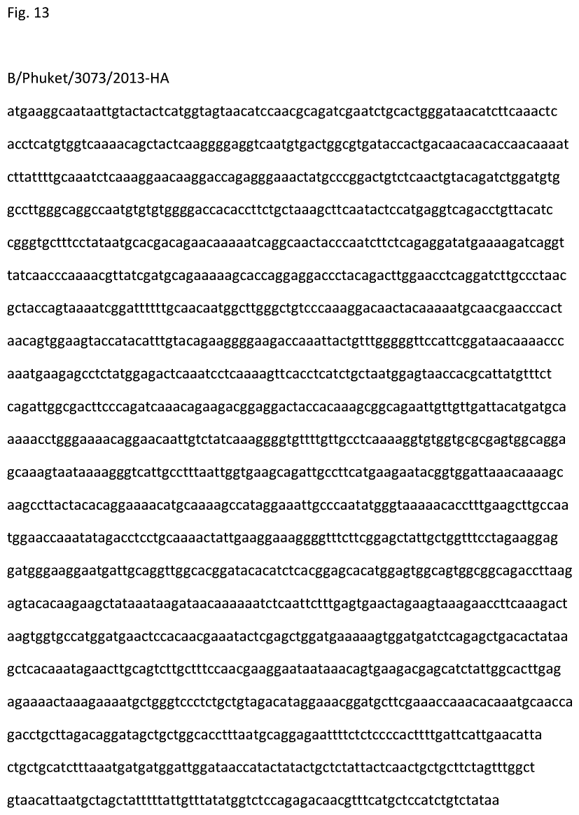

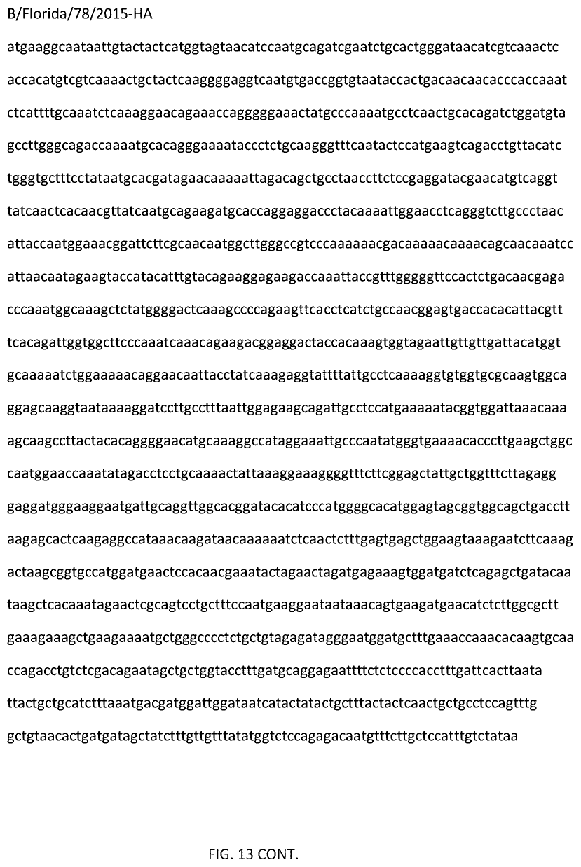

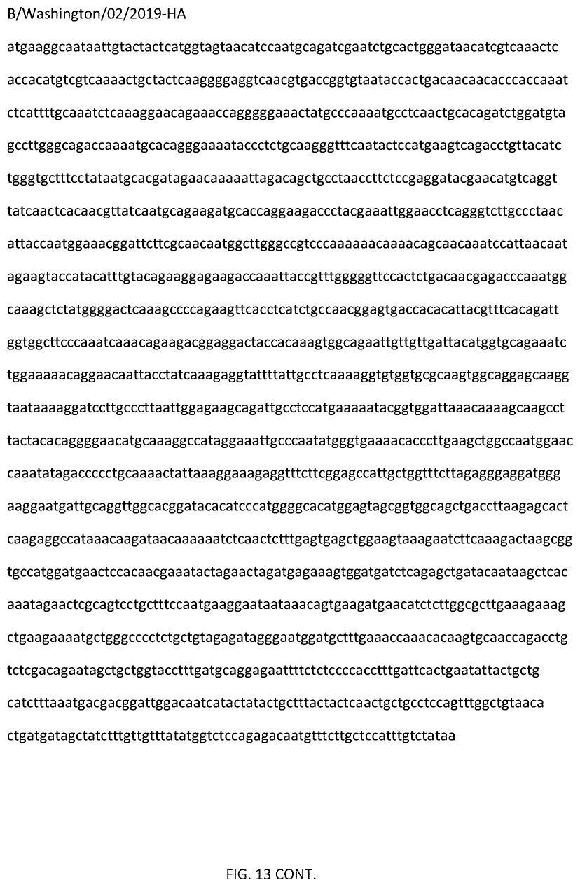

To this end, all available IBV HA sequences were downloaded from GISAID (>52,000) and a phylogenetic tree of the >8,000 unique amino acid sequences was generated by using RaxML. Using this tree, all known IBV HA sequences were classified as either ‘Ancestral’, ‘Victoria’ or ‘Yamagata’ (FIG. 6). In parallel, more than 130 IBVs were obtained. Roughly 40 of these viruses were isolated before the separation of IBVs into the Victoria and Yamagata lineages. The remaining about 90 span all decades since the 1980's, are derived from both lineages, and many of them have been exclusively passaged in cultured cells, ruling out the possibility of egg-adapting mutations that may alter their antigenic properties. Based on the phylogenetic tree, several viruses isolated before the separation of the lineages, and several viruses from each lineage spanning several decades and including major sublineages (these numbers were chosen based on experience with H1N1, H3N2, and H5 antigenic maps), were selected. The selected viruses were derived from a repository, or the HA (and NA) genes were synthesized using commercial services, and IBVs were generated using an established reverse genetics protocol. In addition, ferret sera were generated against several of the IBV HA mutants that gained reactivity with sera raised against a virus from the other lineage.

Over 25 ferret antisera suitable for the development of an antigenic map were generated. For the selected wild-type and mutant viruses, ferrets were immunized intranasally with 10⁶ pfu of live virus. Three weeks later, serum samples were collected and tested by hemagglutination assays for serum antibody titers to the homologous virus. Depending on the HI titers, ferrets may be boosted with adjuvanted, beta-propiolactone-inactivated virus. Three weeks later, sera are collected and ferrets are euthanized.

Next, HI assays are performed to measure the HI titers of all viruses (i.e., wild-type viruses representing both IBV lineages and mutant viruses) against all ferret sera. Based on these titers, the antigenic distances among the viruses can be calculated using the equation Dᵢⱼ=bⱼ−log₂, where Dᵢⱼ is the target distance between virus i and serum j, Hᵢⱼ is the titer of virus i against serum j, and bⱼ is the log₂ value of the highest titer against serum j. The error is calculated based on how well the distance between an antigen and antiserum in an antigenic map reflects the titer measured between them (in an ideal map, the antigen and its homologous antiserum should be in the same position). The positions of all antigens and antisera are ranked using a gradient descent algorithm to produce a final configuration of antigens and antisera with the lowest error.

Existing software, and web platforms (e.g., https://acmacs-web.antigenic-cartography.org), make generating initial antigenic maps relatively straightforward. However, additional analyses may be required to assess the quality of the maps and identify potentially poorly coordinated regions. These include: map distance versus target distance plots (in which points should be scattered with low variation around the line x=y); computing error lines on antisera and antigens, which indicate how far points are away from their ideal locations; and plotting constant force loci (or ‘blobs’), which indicate the region a point may move to if the error is increased by a fixed amount.

The goal is to generate viruses with high reactivity to both IBV lineages, which are antigenically distinct. Such mutants may not fit well in an antigenic map, e.g., by pulling together viruses that should ideally be positioned far apart in antigenic space. For example, maps made with and without mutant viruses are superimposed, to see how the mutants distort the map, and map errors specific to particular mutants are investigated by showing error lines.

Determining appropriate map dimensionality is a step in antigenic cartography. More dimensions may be better able to accommodate more data set variation. Maps are generated in different dimensions and the root mean square error (i.e., the difference between the ideal map location based on HI titers and the actual map location) is calculated for each data point.

At the end a robust IBV antigenic map is established. The antigenic map allows for prioritizing IBV HA mutants for further testing. Specifically, more central IBV HA mutants (located in-between both lineages; FIG. 2) are prioritized over less central mutants. The top IBV HA mutants are then further investigated.

Assess the Immunogenicity of Influenza B Candidate Vaccine Viruses

The immunogenicity of an antigen, i.e., its ability to induce immune responses, underlies the protective efficacy of a vaccine (see FIG. 4). Preliminary studies showed that the immunogenicity of IAV HAs can be increased considerably through the addition of Alhydrogel or by presenting HAs on nanoparticles in combination with adjuvant. The immunogenicity of IBV recombinant HA mutants adjuvanted with Alhydrogel (because recombinant, recombinant HA is already used as an influenza vaccine) are tested, or of nanoparticles and adjuvanted with Alhydrogel. These studies are conducted with recombinant HA to eliminate the contribution of other viral proteins to immune responses.

### Immunogenicity of IBV HA Mutants in Ferrets

Generation of IBV recombinant HA mutants. To generate secreted, mutant IBV HAs for vaccination studies in ferrets, the five selected IBV recombinant HA mutants are expressed in the mammalian Expi293F cell line (Thermo Fisher Scientific) by transfecting cells with expression plasmids encoding the respective recombinant HA. In addition, four secreted control HAs encoding wild-type Florida/Vic, Washington/Vic, Phuket/Yam, and B/Yamagata/1/73 HA (the latter represents an ancestral IBV and was used for the development of our high-yield IBV backbone, are expressed. Specifically, the HA ectodomains followed by a ‘foldon’ motif to ensure trimerization, and followed by a hexa-histidine-tag (His-tag). At positions equivalent to those described for IAV HA, cysteine residues are introduced to enable the formation of stabilizing disulfide bonds (a strategy commonly used for HA expression). Cells are incubated for 4-5 days to allow for HA expression and secretion into the culture supernatant. IBV recombinant HA proteins are purified from the cell culture supernatant by affinity chromatography with TALON metal affinity resin, using our established protocols. The purified proteins are analyzed by SDS-PAGE and Coomassie blue staining to confirm that the purity is >95%.

Assessment of immunogenicity in ferrets. The IBV recombinant HAs or nanoparticles are mixed at a 1:1 volume ratio with Alhydrogel as recommended by the manufacturer. Six-to-eight-month-old ferrets of both sexes (obtained from Triple F Farm), three animals per group, are immunized intramuscularly with 15 μg of each of the selected IBV HA mutants and the HAs of the four control viruses (Phuket/Yam, Florida/Vic, Washington/Vic, B/Yamagata/1/73). Three weeks later, serum samples are collected and HI assays performed alongside reference viruses representing both IBV lineages and ancestral viruses. The resulting HI data is integrated into the antigenic map to assess the antigenic location of the mutant viruses relative to viruses of the Victoria- and Yamagata-lineages. Selected IBV HA mutants likely elicit sera that react with viruses from both IBV lineages; the addition of Alhydrogel, or the addition of Alhydrogel combined with the multivalent presentation of IBV HA mutants on nanoparticles may elicit high antibodies titers that provide a large range of reactivity to confer protection against viruses of both lineages.

Ferrets are not thoroughly inbred, and their MHC locus is not particularly well-characterized, so each ferret's individual response is characterized rather than relying on pre-determined epitope multimers. Overlapping peptides corresponding to the vaccine antigens (17mers, overlapping by 12) are synthesized and used in intracellular cytokine assays to measure peptide specific responses. Responses are detected using a combination of IFN-γ and TNF production, in combination with co-staining with CD4 and CD8 antibodies. All of these antibodies are available and the ferret ICS assay has been validated.

PBMCs matching the time points taken above for serology are analyzed for cellular responses. Additionally, a subset of ferrets immunized with the leading candidates based on serology will be euthanized and sampled for spleen and draining lymph nodes. Ferrets are selected that mounted relatively poorer responses either in magnitude or antigenic breadth. A correlational analysis is performed to associate the specificity of the T cell responses (as defined as conserved vs. variable regions) with the specificity of the antibody responses defined above (defined as breadth of antigenic reactivity). Additionally, it is determined if there are associations between the magnitude and phenotype of T cell responses (defined for CD4 T cells as differentiation state (T effector vs. T follicular helper) and by polyfunctional cytokine production with the magnitude of the antibody responses. A combination of non-parametric correlation (Spearman's) and linear and logistic regression modeling is used to identify the most predictive cellular correlates of potent antibody responses. Both CD4 and CD8 T cell responses are likely elicited, and CD4 responses in particular are correlated with the magnitude of the antibody response.

Whole-Genome-Fragment-Phage-Display-Libraries to Identify Conserved Epitopes that Elicit Broadly Reactive Antibodies

One goal is to identify antigens that elicit antibodies directed at conserved epitopes (in addition to the already know conserved epitopes in the HA stem region) that possess broadly neutralizing activity. To determine whether the IBV HA mutants elicit antibodies that react with such epitopes, “whole-genome-fragment-phage-display-libraries” (GFPDLs) are generated and screened, a technique that maps the immunogenic epitopes of influenza, Ebola, RSV, and SARS-CoV-2 viral antigens. Briefly, phage libraries are generated that express short (50-300 bp) and long (300-1000 bp) sequences of the IBV HA mutants to ensure an unbiased representation of all possible linear and conformation-dependent epitopes across the IBV HA. The IBV-HA-GFPDLs contain large numbers of phages for an unbiased representation of all possible epitopes across the IBV HA. Affinity selection is carried out by incubating the IBV-HA-GFPDLs with selected ferret sera and protein A/G resin. The bound phages are eluted and amplified. Their IBV HA inserts are sequenced, and the IBV HA sequences aligned to the full-length IBV HA sequences to identify the regions in the IBV HAs that are immunogenic. As an example, some broadly reactive sera may target the vestigial esterase domain, which is relatively conserved and immunogenic. Thus, antigens eliciting broadly reactive antibodies targeting the vestigial esterase domain may be attractive candidates for broadly protective IBV vaccines.

Assess the Protective Efficacy of Influenza B Candidate Vaccine Viruses

After establishing the immunogenicity of IBV HA mutants, top candidates are tested for their protective efficacy against IBVs representing both circulating lineages and ancestral viruses.

To test the protective efficacy of the top IBV HA mutants, groups of six animals of both sexes are immunized with the selected antigen, using the strategy that elicited the broadest antibody repertoire (e.g., recombinant HA adjuvanted with Alhydrogel, or HA presented on nanoparticles adjuvanted with Alhydrogel). Control animals are vaccinated with wild-type Florida/Vic or Phuket/Yam recombinant HA adjuvanted with Alhydrogel, or HA adjuvanted and presented on nanoparticles with HA. Three weeks after immunization, serum samples are collected to test the antibody titers by performing HI assays with homologous and heterologous viruses. If robust antibody titers are detected, e.g., a titer of 80 or more against viruses from both lineages, the animals are challenged by intranasal infection with 10⁶ pfu of IBVs possessing the HA and NA of Florida/Vic, Washington/Vic, Phuket/Yam, or B/Yamagata/1/73 in the genetic background of the high-yield IBV backbone. Three animals each are euthanized on days 3 and 6, respectively, to determine virus titers in the respiratory organs, and pathological and immunohistochemistry studies are performed. Additionally, cellular responses (CD4 and CD8) against the vaccine antigens, and against the challenge viruses, are assessed using whole virus ICS assays. These analyses are performed in the spleen, lung tissue, bronchoalveolar lavage, and mediastinal lymph node. The cellular response characterization to individual proteins may be refined using peptide pools corresponding to the major influenza proteins. These assays reveal whether vaccination boosts recall cellular responses and biases the recall response towards epitopes conserved from the vaccine. A similar analytical approach is taken as discussed above, with diverse features of the cellular response (magnitude, differentiation state, effector function, breadth) associated with features of the antibody response and protective efficacy (including viral titer reduction). Linear and logistic regression modeling is used to determine cellular correlates of protection.

The wild-type HA antigens (encoding Florida/Vic, Washington/Vic, and Phuket/Yam HAs) likely provide protection against challenge with the homologous virus, but no or limited protection against a virus of the other IBV lineage. In contrast, IBV HA vaccine candidates provide protection against all challenge viruses, although protection may not be sterilizing (i.e., the vaccine candidates may not prevent the replication of the heterologous challenge viruses), but are expected to reduce the virus titers compared to those in animals vaccinated with the wild-type antigen. Such a finding demonstrates the feasibility of our conceptually new approach for the development of broadly protective IBV vaccines.

### Example 11

Different strategies were tested to obtain ‘in between’ influenza B viruses. The most effective strategy was to create libraries where the influenza virus HA substitutions at residues that varied between the Yamagata and Victoria lineages, e.g., from 1 up to 38 substitutions. For example, the HA1 sequences of Florida (Vic) and Phuket (Yam) viruses were compared and 38 amino acid differences in HA1 were noted. Libraries were prepared encoding each of the parental amino acids at each of the 38 positions. See below for an example.

| | Amino Acid Position | | | |
|---|---|---|---|---|
| | 40 | 48 | 56 | 71 |
| Florida (Vic) | H | E | K | K |
| Phuket (Yam) | Y | R | D | M |
| Library | H, Y | E, R, K*, G* | K, D, N*, E* | K, M |

*There is no codon encoding only the two parental amino acids; thus, additional amino acids are encoded at this position

Libraries were also generated that represent the amino acid differences between Washington (Vic) and Phuket (Yam) HA. These libraries encode only the two parental amino acids at the targeted positions (no additional, unwanted amino acids are encoded).

TABLE 1
HI titers of wild-type and mutant Phuket/Yam and Florida/Vic
viruses against Phuket/Yam and Florida/Vic ferret Sera

| | | Ferret serum | |
|---|---|---|---|
| | | Anti-Phuket/ | Anti-Florida/ |
| | Virus Name | Yam | Vic |
| Wild-type | B/Phuket/3073/2013 | 320 | 80 |
| Wild-type | B/Florida/78/2015 | 20 | 5120 |
| Phuket/Yam HA | Plunket/Yam Mutant #1 | 320 | 320 |
| backbone | Plunket/Yam Mutant #2 | 80 | 320 |
| | Plunket/Yam Mutant #3 | 80 | 320 |
| | Plunket/Yam Mutant #4 | 160 | 320 |
| | Plunket/Yam Mutant #5 | 80 | 320 |
| | Plunket/Yam Mutant #6 | 160 | 320 |
| | Plunket/Yam Mutant #7 | 160 | 640 |
| | Plunket/Yam Mutant #8 | 80 | 640 |
| Florida/Vic HA | Florida/Vic Mutant #1 | 160 | 2560 |
| backbone | Florida/Vic Mutant #2 | 80 | 2560 |
| | Florida/Vic Mutant #3 | 160 | 2560 |
| | Florida/Vic Mutant #4 | 320 | 5120 |
| | Florida/Vic Mutant #5 | 80 | 1280 |
| | Florida/Vic Mutant #6 | 160 | 5120 |
| | Florida/Vic Mutant #7 | 160 | 5120 |
| | Florida/Vic Mutant #8 | 160 | 1280 |
| | Florida/Vic Mutant #9 | 80 | 1280 |
| | Florida/Vic Mutant #10 | 160 | 1280 |
| | Florida/Vic Mutant #11 | 80 | 1280 |

TABLE 2
Preboost data

| | | | HI data | | |
|---|---|---|---|---|---|
| | | | | B/Washington/02/ | B/Phuket/3073/ |
| Map | | | Homologous | 2019 (Victoria- | 2013 (Yamagata- |
| location | Antigen | Ferret# | virus | lineage) | lineage) |
| Center | PhuketHA1- | 5840 | 1280 | 20 | 20 |
| | PhuketHA2- | | | | |
| | WashingtonNA- | | | | |
| | 76 | | | | |
| Vic- | WashingtonHA1- | 5738 | 640 | 80/160 | 160 |
| virus | PhuketHA2- | | | | |
| moved | PhuketNA-21 | | | | |
| to Yam- | | | | | |
| lineage | | | | | |
| Center | WashingtonHA1- | 5846 | 640 | 20 | 160 |
| | PhuketHA2- | | | | |
| | PhuketNA-29 | | | | |
| Center | PhuketHA1- | 5843 | 320 | 10 | 160 |
| | WashingtonHA2- | | | | |
| | PhuketNA-76 | | | | |
| Center | PhuketHA1- | 5737 | 640 | 20 | 80 |
| | WashingtonHA2- | | | | |
| | PhuketNA-40 | | | | |
| Center | PhuketHA1- | 6393 | 2560 | 20 | 320 |
| | WashingtonHA2- | | | | |
| | PhuketNA-73 | | | | |
| Center | PhuketHA1- | 5734 | 640 | 160 | 20 |
| | PhuketHA2- | | | | |
| | WashingtonNA- | | | | |
| | 46 | | | | |
| Center | WashingtonHA1- | 5845 | 640 | 160 | 20 |
| | PhuketHA2- | | | | |
| | WashingtonNA-9 | | | | |
| Vic- | WashingtonHA1- | 5733 | 640 | 160 | 20 |
| virus | PhuketHA2- | | | | |
| moved | WashingtonNA- | | | | |
| to Yam- | 10 | | | | |
| lineage | | | | | |

### Example III

Library, 1, 2, 3, and 4; Mutated HA1, Washington (Vic);
No. of picked and sequenced plaques, 89 plaques, 20 plaques, 47 plaques, and 7 plaques;
No. of well sequenced plaques, 86 plaques, 18 plaques, 39 plaques, and 6 plaques;
No. of unique genotype of plaques, 15 plaques, 7 plaques, 3 plaques, and 4 plaques
Library, 1, 2, 3, and 4; Mutated HA1, Phuket (Yam); HA2, Phuket (Yam), and Washington (Vic); NA, Phuket (Yam), Washington (Vic), Phuket (Yam), and Washington (Vic);
No. of picked and sequenced plaques, 96 plaques, 96 plaques, 96 plaques, and 96 plaques;
No. of well sequenced plaques, 89 plaques, 88 plaques, 89 plaques, and 85 plaques.

### Example IV

HA1 AA sequences of B/Phuket/3073/2013 (Yam) and B/Washington/02/2019 (Vic) were compared. The two viruses differ at 37 amino acid (AA) positions in HA1. Compared to Phuket, the Washington strain has 2 amino acid deletions.

Libraries were prepared that encoded the Phuket or Washington AA at each of the 37 positions.

Washington virus libraries resulted in titers that were low. Virus plaques were picked without antigenic selection.
Library, 1, 2, 3, 4, and Total; Mutated HA1 and Washington (Vic); HA2, Phuket (Yam), and Washington (Vic): NA, Phuket (Yam), Washington (Vic), Phuket (Yam), and Washington (Vic)
No. of picked and sequenced plaques, 89 plaques, 20 plaques, 47 plaques, 7 plaques, and 163 plaques;
No. of well sequenced plaques, 86 plaques, 18 plaques, 39 plaques, 6 plaques, and 149 plaques;
No. of unique genotype of plaques, 15 plaques, 7 plaques, 3 plaques, 4 plaques, and 29 plaques.

Library, 1, 2, 3, 4, and Total; Mutated HA1 and Phuket (Yam); HA2, Phuket (Yam), and Washington (Vic); NA, Phuket (Yam), Washington (Vic), Phuket (Yam), and Washington (Vic);
No. of picked and sequenced plaques, 96 plaques, 96 plaques, 96 plaques, 96 plaques, and 384 plaques;
No. of well sequenced plaques, 89 plaques, 88 plaques, 89 plaques, 85 plaques, and 351 plaques;
No. of unique genotype of plaques, 43 plaques, 60 plaques, 48 plaques, 37 plaques, and 188 plaques.

Anti-Phuket (1231) and anti-Washington (F5099) ferret sera was used to screen the 188 Phuket-based and 29 Washington-based mutants.

If the HI data of a mutant: >=40 against both of anti-Washington anti-Phuket sera, the virus may be an “in between” virus

### Flu B Mutants

TABLE 3

| | | | | | | 39 different amino acids (including two | | | | | | | | | | | | | | |
|---|---|---|---|---|---|---|---|---|---|---|---|---|---|---|---|---|---|---|---|---|
| | | | | | | a.a. deletion in Washington/02 HA1) | | | | | | | | | | | | | | |
| | | | | | | between Phuket/3073 and Washington/02 | | | | | | | | | | | | | | |
| | | | Interesting | | | | | | | | | | | | | | | | | |
| | | | candidates* | | | 1 | 2 | 3 | 4 | 5 | 6 | 7 | 8 | 9 | 10 | 11 | 12 | 13 | 14 | 15 |
| | | | Anti- | Anti- | | 40 | 4 | 5 | 7 | 7 | 7 | 7 | 8 | 8 | 11 | 11 | 12 | 6 | 12 | 13 |
| | | | Phuket | Wash | Map | | 8 | 6 | 1 | 3 | 5 | 6 | 0 | 1 | 6 | 7 | 2 | 12 | 9 | 3 |
| Wild | | B/Phuket/3073/20-13- | 160 | 20 | | Y | R | D | M | V | T | T | K | A | K | I | Q | D | K | G |
| type | | HYYam | | | | | | | | | | | | | | | | | | |
| | | B/Washington/02/201 | <20 | 10240 | | H | E | K | K | T | K | I | R | V | H | V | H | N | D | R |
| | | 9-HYYam | | | | | | | | | | | | | | | | | | |
| Control | | PhuketHA1- | 160 | 40 | | Y | R | D | M | V | T | T | K | A | K | I | Q | D | K | G |
| | | WashingtonHA2- | | | | | | | | | | | | | | | | | | |
| | | WashingtonNA | | | | | | | | | | | | | | | | | | |
| | | PhuketHA1- | 160 | 20 | | Y | R | D | M | V | T | T | K | A | K | I | Q | D | K | G |
| | | WashingtonHA2- | | | | | | | | | | | | | | | | | | |
| | | PhuketNA | | | | | | | | | | | | | | | | | | |
| | | WashingtonHA1- | 20 | 10240 | | H | E | K | M | T | K | I | R | V | H | V | H | N | D | R |
| | | PhuketHA2- | | | | | | | | | | | | | | | | | | |
| | | WashingtonNA | | | | | | | | | | | | | | | | | | |
| | | WashingtonHA1- | 20 | 10240 | | H | E | K | K | T | K | I | R | V | H | V | H | N | D | R |
| | | PhuketHA2-PhuketNA | | | | | | | | | | | | | | | | | | |
| Mutant | 1 | PhuketHA1- | 320 | 2560 | | H | E | . | . | T | . | I | R | . | H | . | . | . | . | R |
| | | WashingtonHA2- | | | | | | | | | | | | | | | | | | |
| | | WashingtonNA-29 | | | | | | | | | | | | | | | | | | |
| | 2 | PhuketHA1- | 80 | 1280 | | H | . | K | . | . | . | I | R | V | H | . | H | N | D | . |
| | | PhuketHA2- | | | | | | | | | | | | | | | | | | |
| | | WashingtonNA-60 | | | | | | | | | | | | | | | | | | |
| | 3 | PhuketHA1- | 160 | 640 | | . | . | . | . | . | K | . | . | V | H | V | . | . | . | . |
| | | PhuketHA2- | | | | | | | | | | | | | | | | | | |
| | | WashingtonNA-32 | | | | | | | | | | | | | | | | | | |
| | 4 | PhuketHA1- | 160 | 640 | | . | . | . | . | T | K | I | R | . | H | . | H | N | . | . |
| | | PhuketHA2- | | | | | | | | | | | | | | | | | | |
| | | WashingtonNA-7 | | | | | | | | | | | | | | | | | | |
| | 5 | PhuketHA1- | 320 | 320 | | . | . | . | . | . | . | . | R | V | . | V | H | . | . | . |
| | | WashingtonHA2- | | | | | | | | | | | | | | | | | | |
| | | WashingtonNA-11 | | | | | | | | | | | | | | | | | | |
| | 6 | PhuketHA1- | 160 | 320 | | H | . | K | . | . | K | I | R | V | . | . | H | N | . | . |
| | | PhuketHA2- | | | | | | | | | | | | | | | | | | |
| | | PhuketNA- | | | | | | | | | | | | | | | | | | |
| | | 32 | | | | | | | | | | | | | | | | | | |
| | 7 | PhuketHA1- | 160 | 320 | | . | E | . | . | . | . | . | R | V | H | . | H | . | . | . |
| | | WashingtonHA2- | | | | | | | | | | | | | | | | | | |
| | | PhuketNA-32 | | | | | | | | | | | | | | | | | | |
| | 8 | PhuketHA1- | 80 | 320 | | H | E | K | . | T | K | . | . | . | H | . | . | . | . | . |
| | | PhuketHA2- | | | | | | | | | | | | | | | | | | |
| | | WashingtonNA-46 | | | | | | | | | | | | | | | | | | |
| | 9 | PhuketHA1- | 80 | 320 | | H | E | . | K | . | K | . | R | V | H | V | H | . | . | . |
| | | PhuketHA2- | | | | | | | | | | | | | | | | | | |
| | | WashingtonNA-76 | | | | | | | | | | | | | | | | | | |
| | 10 | PhuketHA1- | 160 | 160 | | H | E | . | . | . | K | . | . | V | H | V | . | N | D | . |
| | | PhuketHA2- | | | | | | | | | | | | | | | | | | |
| | | PhuketNA- | | | | | | | | | | | | | | | | | | |
| | | 51 | | | | | | | | | | | | | | | | | | |
| | 11 | PhuketHA1- | 160 | 160 | | H | . | . | . | T | . | . | R | . | . | . | H | N | D | . |
| | | PhuketHA2- | | | | | | | | | | | | | | | | | | |
| | | WashingtonNA-33 | | | | | | | | | | | | | | | | | | |
| | 12 | PhuketHA1- | 160 | 160 | | H | . | . | . | . | K | I | . | V | H | . | H | N | D | . |
| | | WashingtonHA2- | | | | | | | | | | | | | | | | | | |
| | | PhuketNA-73 | | | | | | | | | | | | | | | | | | |
| | 13 | PhuketHA1- | 160 | 160 | | . | E | K | K | . | K | I | . | . | . | V | . | N | . | . |
| | | WashingtonHA2- | | | | | | | | | | | | | | | | | | |
| | | PhuketNA-76 | | | | | | | | | | | | | | | | | | |
| | 14 | PhuketHA1- | 160 | 160 | | H | . | K | . | T | K | I | R | . | H | . | . | N | D | . |
| | | WashingtonHA2- | | | | | | | | | | | | | | | | | | |
| | | WashingtonNA-18 | | | | | | | | | | | | | | | | | | |
| | 15 | PhuketHA1- | 160 | 80 | | H | . | K | . | T | . | I | . | V | . | . | . | N | D | . |
| | | PhuketHA2- | | | | | | | | | | | | | | | | | | |
| | | PhuketNA-93 | | | | | | | | | | | | | | | | | | |
| | 16 | PhuketHA1- | 160 | 80 | | . | E | K | K | T | K | . | . | V | H | . | H | . | D | . |
| | | PhuketHA2- | | | | | | | | | | | | | | | | | | |
| | | WashingtonNA-25 | | | | | | | | | | | | | | | | | | |
| | 17 | PhuketHA1- | 160 | 80 | | . | E | . | K | T | . | . | R | V | . | . | . | N | D | . |
| | | WashingtonHA2- | | | | | | | | | | | | | | | | | | |
| | | PhuketNA-40 | | | | | | | | | | | | | | | | | | |
| | 18 | PhuketHA1- | 160 | 80 | | H | E | . | K | T | K | . | . | V | . | V | . | N | D | . |
| | | WashingtonHA2- | | | | | | | | | | | | | | | | | | |
| | | WashingtonNA-4 | | | | | | | | | | | | | | | | | | |
| | 19 | PhuketHA1- | 160 | 80 | | H | E | . | . | . | . | . | . | V | H | . | H | N | D | R |
| | | WashingtonHA2- | | | | | | | | | | | | | | | | | | |
| | | WashingtonNA-92 | | | | | | | | | | | | | | | | | | |
| | 20 | PhuketHA1- | 80 | 80 | | . | E | . | K | . | . | I | . | V | H | . | . | . | . | . |
| | | PhuketHA2- | | | | | | | | | | | | | | | | | | |
| | | WashingtonNA-49 | | | | | | | | | | | | | | | | | | |
| | 21 | PhuketHA1- | 80 | 80 | | H | E | K | . | T | K | . | . | V | H | . | H | N | . | . |
| | | WashingtonHA2- | | | | | | | | | | | | | | | | | | |
| | | PhuketNA-13 | | | | | | | | | | | | | | | | | | |
| | 22 | PhuketHA1- | 80 | 80 | | . | E | K | . | . | . | I | R | . | H | V | . | N | D | R |
| | | WashingtonHA2- | | | | | | | | | | | | | | | | | | |
| | | PhuketNA-12 | | | | | | | | | | | | | | | | | | |
| | 23 | PhuketHA1- | 80 | 80 | | H | . | K | K | T | . | . | R | V | . | . | H | . | D | . |
| | | WashingtonHA2- | | | | | | | | | | | | | | | | | | |
| | | PhuketNA-34 | | | | | | | | | | | | | | | | | | |
| | 24 | PhuketHA1- | 80 | 80 | | . | E | K | . | T | K | . | R | . | H | . | . | N | . | . |
| | | WashingtonHA2- | | | | | | | | | | | | | | | | | | |
| | | PhuketNA-64 | | | | | | | | | | | | | | | | | | |
| | 25 | PhuketHA1- | 80 | 80 | | H | . | K | . | . | . | I | R | V | H | V | . | N | D | R |
| | | WashingtonHA2- | | | | | | | | | | | | | | | | | | |
| | | PhuketNA-7 | | | | | | | | | | | | | | | | | | |
| | 26 | WashingtonHA1- | 160 | 10240 | | . | E | K | K | T | . | . | . | . | H | V | . | . | D | . |
| | | PhuketHA2- | | | | | | | | | | | | | | | | | | |
| | | PhuketNA- | | | | | | | | | | | | | | | | | | |
| | | 87 | | | | | | | | | | | | | | | | | | |
| | 27 | WashingtonHA1- | 80 | 20480 | | H | . | K | . | T | . | . | . | V | H | V | . | . | D | R |
| | | PhuketHA2- | | | | | | | | | | | | | | | | | | |
| | | PhuketNA- | | | | | | | | | | | | | | | | | | |
| | | 5 | | | | | | | | | | | | | | | | | | |
| | 28 | WashingtonHA1- | 80 | 2560 | | H | E | K | K | T | K | . | . | V | H | . | . | N | . | . |
| | | PhuketHA2- | | | | | | | | | | | | | | | | | | |
| | | PhuketNA- | | | | | | | | | | | | | | | | | | |
| | | 29 | | | | | | | | | | | | | | | | | | |
| | 29 | WashingtonHA1- | 80 | 1280 | | H | . | K | K | T | . | . | . | V | H | . | . | N | D | R |
| | | PhuketHA2- | | | | | | | | | | | | | | | | | | |
| | | PhuketNA- | | | | | | | | | | | | | | | | | | |
| | | 21 | | | | | | | | | | | | | | | | | | |
| | 30 | WashingtonHAI- | 320 | 640 | | H | . | . | . | . | . | I | . | . | . | V | . | N | . | . |
| | | PhuketHA2- | | | | | | | | | | | | | | | | | | |
| | | WashingtonNA-10 | | | | | | | | | | | | | | | | | | |
| | 31 | WashingtonHA1- | 80 | 5120 | | . | E | K | K | T | K | I | R | . | . | . | . | . | D | . |
| | | PhuketHA2- | | | | | | | | | | | | | | | | | | |
| | | WashingtonNA-7 | | | | | | | | | | | | | | | | | | |
| | 32 | WashingtonHA1- | 80 | 160 | | . | E | . | . | . | . | I | R | . | H | . | . | . | . | . |
| | | PhuketHA2- | | | | | | | | | | | | | | | | | | |
| | | WashingtonNA-9 | | | | | | | | | | | | | | | | | | |
| | 33 | WashingtonHA1- | 160 | 5120 | | H | . | . | . | . | . | I | R | . | . | V | H | . | D | . |
| | | WashingtonHA2- | | | | | | | | | | | | | | | | | | |
| | | WashingtonNA-7 | | | | | | | | | | | | | | | | | | |
| | | | Interesting | | | | | | | | | | | | | | | | | |
| | | | candidates* | | | 16 | 17 | 18 | 19 | 20 | 21 | 22 | 23 | 24 | 25 | 26 | 27 | 28 | 29 | |
| | | | Anti- | Anti- | | 136 | 13 | 14 | 14 | 14 | 15 | 16 | 16 | 16 | 16 | 16 | 17 | 19 | 20 | |
| | | | Phuket | Wash | Map | | 7 | 6 | 8 | 9 | 0 | 2 | 3 | 4 | 5 | 7 | 4 | 7 | 1 | |
| Wild | | B/Phuket/3073/20- | 160 | 20 | | R | L | A | S | K | I | K | D | N | Y | N | V | K | K | |
| type | | 13-HYYam | | | | | | | | | | | | | | | | | | |
| | | B/Washington/02/20 | <20 | 10240 | | E | I | I | N | G | N | Δ | Δ | K | N | T | I | E | A | |
| | | 19-HYYam | | | | | | | | | | | | | | | | | | |
| Control | | PhuketHA1- | 160 | 40 | | R | L | A | S | K | 1 | K | D | N | Y | N | V | K | K | |
| | | WashingtonHA2- | | | | | | | | | | | | | | | | | | |
| | | WashingtonNA | | | | | | | | | | | | | | | | | | |
| | | PhuketHA1- | 160 | 20 | | R | L | A | S | K | 1 | K | D | N | Y | N | V | K | K | |
| | | WashingtonHA2- | | | | | | | | | | | | | | | | | | |
| | | PhuketNA | | | | | | | | | | | | | | | | | | |
| | | WashingtonHA1- | 20 | 10240 | | F | I | I | N | G | N | Δ | Δ | K | N | T | I | E | A | |
| | | PhuketHA2- | | | | | | | | | | | | | | | | | | |
| | | WashingtonNA | | | | | | | | | | | | | | | | | | |
| | | WashingtonHA1- | 20 | 10240 | | F | I | I | N | G | N | Δ | Δ | K | N | T | I | E | A | |
| | | PhuketHA2- | | | | | | | | | | | | | | | | | | |
| | | PhuketNA | | | | | | | | | | | | | | | | | | |
| Mutant | 1 | PhuketHA1- | 320 | 2560 | | E | . | . | . | . | . | K | D | K | N | T | . | . | A | |
| | | WashingtonHA2- | | | | | | | | | | | | | | | | | | |
| | | WashingtonNA-29 | | | | | | | | | | | | | | | | | | |
| | 2 | PhuketHA1- | 80 | 1280 | | E | I | I | . | . | N | K | D | K | N | . | . | E | . | |
| | | PhuketHA2- | | | | | | | | | | | | | | | | | | |
| | | WashingtonNA-60 | | | | | | | | | | | | | | | | | | |
| | 3 | PhuketHA1- | 160 | 640 | | E | . | . | N | . | N | K | D | K | N | T | I | E | . | |
| | | PhuketHA2- | | | | | | | | | | | | | | | | | | |
| | | WashingtonNA-32 | | | | | | | | | | | | | | | | | | |
| | 4 | PhuketHA1- | 160 | 640 | | E | . | . | N | . | . | K | D | K | N | . | I | E | A | |
| | | PhuketHA2- | | | | | | | | | | | | | | | | | | |
| | | WashingtonNA-7 | | | | | | | | | | | | | | | | | | |
| | 5 | PhuketHA1- | 320 | 320 | | E | I | . | . | . | . | K | D | K | N | . | I | . | A | |
| | | WashingtonHA2- | | | | | | | | | | | | | | | | | | |
| | | WashingtonNA-11 | | | | | | | | | | | | | | | | | | |
| | 6 | PhuketHA1- | 160 | 320 | | E | . | I | N | . | . | K | D | . | . | T | I | E | A | |
| | | PhuketHA2- | | | | | | | | | | | | | | | | | | |
| | | PhuketNA-32 | | | | | | | | | | | | | | | | | | |
| | 7 | PhuketHA1- | 160 | 320 | | E | I | . | N | . | . | K | D | K | N | . | I | E | . | |
| | | WashingtonHA2- | | | | | | | | | | | | | | | | | | |
| | | PhuketNA-32 | | | | | | | | | | | | | | | | | | |
| | 8 | PhuketHA1- | 80 | 320 | | E | . | I | . | . | N | K | D | K | . | T | . | E | A | |
| | | PhuketHA2- | | | | | | | | | | | | | | | | | | |
| | | WashingtonNA-46 | | | | | | | | | | | | | | | | | | |
| | 9 | PhuketHA1- | 80 | 320 | | E | I | . | N | G | . | K | D | K | N | . | I | . | A | |
| | | PhuketHA2- | | | | | | | | | | | | | | | | | | |
| | | WashingtonNA-76 | | | | | | | | | | | | | | | | | | |
| | 10 | PhuketHA1- | 160 | 160 | | E | . | I | N | . | . | K | D | K | . | . | I | | A | |
| | | PhuketHA2- | | | | | | | | | | | | | | | | | | |
| | | PhuketNA-51 | | | | | | | | | | | | | | | | | | |
| | 11 | PhuketHA1- | 160 | 160 | | E | . | . | | . | . | K | D | K | . | . | I | E | A | |
| | | PhuketHA2- | | | | | | | | | | | | | | | | | | |
| | | WashingtonNA-33 | | | | | | | | | | | | | | | | | | |
| | 12 | PhuketHA1- | 160 | 160 | | E | I | . | | G | . | K | D | . | . | T | . | . | A | |
| | | WashingtonHA2- | | | | | | | | | | | | | | | | | | |
| | | PhuketNA-73 | | | | | | | | | | | | | | | | | | |
| | 13 | PhuketHA1- | 160 | 160 | | E | . | . | N | . | N | K | D | | . | . | . | E | . | |
| | | WashingtonHA2- | | | | | | | | | | | | | | | | | | |
| | | PhuketNA-76 | | | | | | | | | | | | | | | | | | |
| | 14 | PhuketHA1- | 160 | 160 | | E | I | . | . | . | N | K | D | . | . | . | I | E | A | |
| | | WashingtonHA2- | | | | | | | | | | | | | | | | | | |
| | | WashingtonNA-18 | | | | | | | | | | | | | | | | | | |
| | 15 | PhuketHA1- | 160 | 80 | | E | . | I | . | . | . | K | D | K | N | . | . | E | . | |
| | | PhuketHA2- | | | | | | | | | | | | | | | | | | |
| | | PhuketNA-93 | | | | | | | | | | | | | | | | | | |
| | 16 | PhuketHA1- | 160 | 80 | | E | . | . | . | . | . | K | D | . | . | T | I | . | A | |
| | | PhuketHA2- | | | | | | | | | | | | | | | | | | |
| | | WashingtonNA-25 | | | | | | | | | | | | | | | | | | |
| | 17 | PhuketHA1- | 160 | 80 | | E | I | . | . | . | . | K | D | K | . | . | . | E | . | |
| | | WashingtonHA2- | | | | | | | | | | | | | | | | | | |
| | | PhuketNA-40 | | | | | | | | | | | | | | | | | | |
| | 18 | PhuketHA1- | 160 | 80 | | E | . | . | . | . | N | K | D | . | . | T | I | E | A | |
| | | WashingtonHA2- | | | | | | | | | | | | | | | | | | |
| | | WashingtonNA-4 | | | | | | | | | | | | | | | | | | |
| | 19 | PhuketHA1- | 160 | 80 | | E | . | . | . | . | . | K | D | K | . | T | . | . | A | |
| | | WashingtonHA2- | | | | | | | | | | | | | | | | | | |
| | | WashingtonNA-92 | | | | | | | | | | | | | | | | | | |
| | 20 | PhuketHA1- | 80 | 80 | | E | . | I | . | G | N | K | D | . | N | . | I | E | A | |
| | | PhuketHA2- | | | | | | | | | | | | | | | | | | |
| | | WashingtonNA-49 | | | | | | | | | | | | | | | | | | |
| | 21 | PhuketHA1- | 80 | 80 | | E | . | I | . | . | . | K | D | . | N | . | . | E | . | |
| | | WashingtonHA2- | | | | | | | | | | | | | | | | | | |
| | | PhuketNA-13 | | | | | | | | | | | | | | | | | | |
| | 22 | PhuketHA1- | 80 | 80 | | E | I | . | . | . | . | K | D | . | N | T | I | E | A | |
| | | WashingtonHA2- | | | | | | | | | | | | | | | | | | |
| | | PhuketNA-12 | | | | | | | | | | | | | | | | | | |
| | 23 | PhuketHA1- | 80 | 80 | | . | I | I | . | . | . | K | D | . | N | . | I | E | . | |
| | | WashingtonHA2- | | | | | | | | | | | | | | | | | | |
| | | PhuketNA-34 | | | | | | | | | | | | | | | | | | |
| | 24 | PhuketHA1- | 80 | 80 | | E | . | . | N | . | N | K | D | K | . | . | I | . | A | |
| | | WashingtonHA2- | | | | | | | | | | | | | | | | | | |
| | | PhuketNA-64 | | | | | | | | | | | | | | | | | | |
| | 25 | PhuketHA1- | 80 | 80 | | E | . | I | . | . | N | K | D | . | N | . | . | E | . | |
| | | WashingtonHA2- | | | | | | | | | | | | | | | | | | |
| | | PhuketNA-7 | | | | | | | | | | | | | | | | | | |
| | 26 | WashingtonHA1- | 160 | 10240 | | E | I | . | . | . | . | Δ | Δ | K | . | T | I | . | A | |
| | | PhuketHA2- | | | | | | | | | | | | | | | | | | |
| | | PhuketNA-87 | | | | | | | | | | | | | | | | | | |
| | 27 | WashingtonHA1- | 80 | 20480 | | E | . | . | N | . | N | Δ | Δ | K | N | T | . | E | A | |
| | | PhuketHA2- | | | | | | | | | | | | | | | | | | |
| | | PhuketNA-5 | | | | | | | | | | | | | | | | | | |
| | 28 | WashingtonHA1- | 80 | 2560 | | . | I | I | . | G | . | Δ | Δ | K | N | T | I | E | . | |
| | | PhuketHA2- | | | | | | | | | | | | | | | | | | |
| | | PhuketNA-29 | | | | | | | | | | | | | | | | | | |
| | 29 | WashingtonHA1- | 80 | 1280 | | . | I | . | . | . | . | Δ | Δ | K | N | T | I | E | A | |
| | | PhuketHA2- | | | | | | | | | | | | | | | | | | |
| | | PhuketNA-21 | | | | | | | | | | | | | | | | | | |
| | 30 | WashingtonHA1- | 320 | 640 | | E | I | I | . | . | . | Δ | Δ | K | . | . | I | . | A | |
| | | PhuketHA2- | | | | | | | | | | | | | | | | | | |
| | | WashingtonNA-10 | | | | | | | | | | | | | | | | | | |
| | 31 | WashingtonHA1- | 80 | 5120 | | . | I | . | . | . | . | Δ | Δ | . | N | T | . | E | A | |
| | | PhuketHA2- | | | | | | | | | | | | | | | | | | |
| | | WashingtonNA-7 | | | | | | | | | | | | | | | | | | |
| | 32 | WashingtonHA1- | 80 | 160 | | E | I | I | . | . | N | Δ | Δ | K | . | . | . | E | . | |
| | | PhuketHA2- | | | | | | | | | | | | | | | | | | |
| | | WashingtonNA-9 | | | | | | | | | | | | | | | | | | |
| | 33 | WashingtonHA1- | 160 | 5120 | | . | I | I | . | . | . | Δ | Δ | . | . | T | I | . | A | |
| | | WashingtonHA2- | | | | | | | | | | | | | | | | | | |
| | | WashingtonNA-7 | | | | | | | | | | | | | | | | | | |

Interesting

| | | | candidates* | | | | | | | | | | | | | | |
|---|---|---|---|---|---|---|---|---|---|---|---|---|---|---|---|---|---|
| | | | Anti- | Anti- | | 30 | 31 | 32 | 33 | 34 | 35 | 36 | 37 | 38 | 39 | Unexpected mutant | |
| | | | Phuket | Wash | Map | 202 | 208 | 229 | 232 | 251 | 254 | 261 | 266 | 298 | 312 | 157 | 206 |
| Wild | | B/Phuket/307320/13- | 160 | 20 | | S | N | D | D | M | P | V | V | E | K | A | D |
| type | | HYYam | | | | | | | | | | | | | | | |
| | | B/Washington/02/2019- | <20 | 10240 | | K | K | G | N | V | S | T | I | K | E | . | . |
| | | HYYam | | | | | | | | | | | | | | | |
| Control | | PhuketHA1- | 160 | 40 | | S | N | D | D | M | P | V | V | E | K | A | D |
| | | WashingtonHA2- | | | | | | | | | | | | | | | |
| | | WashingtonNA | | | | | | | | | | | | | | | |
| | | PhuketHA1- | 160 | 20 | | S | N | D | D | M | P | V | V | E | K | A | D |
| | | WashingtonHA2- | | | | | | | | | | | | | | | |
| | | PhuketNA | | | | | | | | | | | | | | | |
| | | WashingtonHA1- | 20 | 10240 | | K | K | G | N | V | S | T | I | K | E | . | . |
| | | PhuketHA2- | | | | | | | | | | | | | | | |
| | | WashingtonNA | | | | | | | | | | | | | | | |
| | | WashingtonHA1- | 20 | 10240 | | K | K | G | N | V | S | T | I | K | E | . | . |
| | | PhuketHA2-PhuketNA | | | | | | | | | | | | | | | |
| | 1 | PhuketHA1- | 320 | 2560 | | K | . | . | . | V | S | . | I | . | E | . | . |
| | | WashingtonHA2- | | | | | | | | | | | | | | | |
| | | WashingtonNA-29 | | | | | | | | | | | | | | | |
| | 2 | PhuketHA1-PhuketHA2- | 80 | 1280 | | K | . | . | N | V | 5 | T | . | . | E | . | . |
| | | WashingtonNA-60 | | | | | | | | | | | | | | | |
| | 3 | PhuketHA1-PhuketHA2- | 160 | 640 | | . | K | . | N | V | S | . | I | . | E | . | Y |
| | | WashingtonNA-32 | | | | | | | | | | | | | | | |
| | 4 | PhuketHA1-PhuketHA2- | 160 | 640 | | K | . | . | N | V | S | . | . | K | | . | . |
| | | WashingtonNA-7 | | | | | | | | | | | | | | | |
| | 5 | PhuketHA1- | 320 | 320 | | K | K | . | N | V | S | T | I | . | . | . | . |
| | | WashingtonHA2- | | | | | | | | | | | | | | | |
| | | WashingtonNA-11 | | | | | | | | | | | | | | | |
| | 6 | PhuketHA1-PhuketHA2- | 160 | 320 | | . | . | G | . | V | S | . | . | . | . | . | . |
| | | PhuketNA-32 | | | | | | | | | | | | | | | |
| | | | 160 | 320 | | K | . | G | N | V | S | . | . | K | E | . | . |
| | 8 | PhuketHA1-PhuketHA2- | 80 | 320 | | K | . | G | . | V | . | T | I | K | E | . | . |
| | | WashingtonNA-46 | | | | | | | | | | | | | | | |
| | 9 | PhuketHA1-PhuketHA2- | 80 | 320 | | K | K | . | . | V | | T | . | . | . | . | . |
| | | WashingtonNA-76 | | | | | | | | | | | | | | | |
| | 10 | PhuketHA1-PhuketHA2- | 160 | 160 | | K | . | G | . | V | . | T | . | . | E | . | . |
| | | PhuketNA-51 | | | | | | | | | | | | | | | |
| | 11 | PhuketHA1-PhuketHA2- | 160 | 160 | | K | . | . | . | . | S | . | I | . | E | . | . |
| | | WashingtonNA-33 | | | | | | | | | | | | | | | |
| | 12 | PhuketHA1- | 160 | 160 | | . | K | . | . | V | . | . | I | K | E | . | . |
| | | WashingtonHA2- | | | | | | | | | | | | | | | |
| | | PhuketNA-73 | | | | | | | | | | | | | | | |
| | 13 | PhuketHA1- | 160 | 160 | | K | . | G | N | V | . | . | . | K | . | . | . |
| | | WashingtonHA2- | | | | | | | | | | | | | | | |
| | | PhuketNA-76 | | | | | | | | | | | | | | | |
| | 14 | PhuketHA1- | 160 | 160 | | K | . | G | N | . | . | . | . | . | . | . | . |
| | | WashingtonHA2- | | | | | | | | | | | | | | | |
| | | PhuketNA-18 | | | | | | | | | | | | | | | |
| | 15 | PhuketHA1- | 160 | 80 | . | . | . | . | N | V | . | T | I | K | . | . | . |
| | | PhuketHA2- | | | | | | | | | | | | | | | |
| | | WashingtonNA-93 | | | | | | | | | | | | | | | |
| | 16 | PhuketHA1-PhuketHA2- | 160 | 80 | | K | K | . | . | V | S | T | . | . | . | . | . |
| | | WashingtonNA-25 | | | | | | | | | | | | | | | |
| | 17 | PhuketHA1- | 160 | 80 | | K | K | G | N | V | S | T | I | K | E | . | . |
| | | WashingtonHA2- | | | | | | | | | | | | | | | |
| | | PhuketNA-40 | | | | | | | | | | | | | | | |
| | 18 | PhuketHA1- | 160 | 80 | | K | K | . | . | V | S | . | I | K | . | . | . |
| | | WashingtonHA2- | | | | | | | | | | | | | | | |
| | | WashingtonNA-4 | | | | | | | | | | | | | | | |
| | 19 | PhuketHA1- | 160 | 80 | | . | . | . | . | . | . | T | I | K | E | T | . |
| | | WashingtonHA2- | | | | | | | | | | | | | | | |
| | | WashingtonNA-92 | | | | | | | | | | | | | | | |
| | 20 | PhuketHA1- | 80 | 80 | | K | . | . | . | V | . | . | . | . | . | . | . |
| | | PhuketHA2- | | | | | | | | | | | | | | | |
| | | WashingtonNA-49 | | | | | | | | | | | | | | | |
| | 21 | PhuketHA1- | 80 | 80 | | K | . | G | N | . | S | . | i | K | E | . | . |
| | | WashingtonHA2- | | | | | | | | | | | | | | | |
| | | PhuketNA-13 | | | | | | | | | | | | | | | |
| | 22 | PhuketHA1- | 80 | 80 | | K | K | G | . | . | . | . | . | . | . | . | . |
| | | WashingtonHA2- | | | | | | | | | | | | | | | |
| | | PhuketNA-12 | | | | | | | | | | | | | | | |
| | 23 | PhuketHA1- | 80 | 80 | | . | . | . | N | . | . | T | . | . | E | . | . |
| | | WashingtonHA2- | | | | | | | | | | | | | | | |
| | | PhuketNA-34 | | | | | | | | | | | | | | | |
| | 24 | PhuketHA1- | 80 | 80 | | . | K | G | . | . | . | T | . | . | . | . | . |
| | | WashingtonHA2- | | | | | | | | | | | | | | | |
| | | PhuketNA-64 | | | | | | | | | | | | | | | |
| | 25 | PhuketHA1- | 80 | 80 | | . | K | . | . | V | S | . | . | K | E | . | . |
| | | WashingtonHA2- | | | | | | | | | | | | | | | |
| | | PhuketNA-7 | | | | | | | | | | | | | | | |
| | 26 | WashingtonHA1- | 160 | 10240 | | K | K | . | N | V | . | T | I | K | E | . | . |
| | | PhuketHA2-PhuketNA- | | | | | | | | | | | | | | | |
| | | 87 | | | | | | | | | | | | | | | |
| | 27 | WashingtonHA1- | 80 | 20480 | | . | . | . | N | . | . | T | I | K | E | . | . |
| | | PhuketHA2-PhuketNA-5 | | | | | | | | | | | | | | | |
| | 28 | WashingtonHA1- | 80 | 2560 | | K | K | . | N | . | . | T | I | . | E | . | . |
| | | PhuketHA2-PhuketNA- | | | | | | | | | | | | | | | |
| | | 29 | | | | | | | | | | | | | | | |
| | 29 | WashingtonHA1- | 80 | 1280 | | . | K | . | N | V | . | T | I | . | . | . | . |
| | | PhuketHA2-PhuketNA- | | | | | | | | | | | | | | | |
| | | 21 | | | | | | | | | | | | | | | |
| | 30 | WashingtonHA1- | 320 | 640 | | . | K | . | . | V | . | . | . | . | . | . | . |
| | | PhuketHA2- | | | | | | | | | | | | | | | |
| | | WashingtonNA-10 | | | | | | | | | | | | | | | |
| | 31 | WashingtonHA1- | 80 | 5120 | | K | K | . | N | V | . | . | I | . | . | . | . |
| | | PhuketHA2- | | | | | | | | | | | | | | | |
| | | WashingtonNA-7 | | | | | | | | | | | | | | | |
| | 32 | WashingtonHA1- | 80 | 160 | | K | K | . | N | . | . | T | . | K | E | . | . |
| | | PhuketHA2- | | | | | | | | | | | | | | | |
| | | WashingtonNA-9 | | | | | | | | | | | | | | | |
| | 33 | WashingtonHA1- | 160 | 5120 | | K | K | | N | V | . | | | K | | . | . |
| | | WashingtonHA2- | | | | | | | | | | | | | | | |
| | | WashingtonNA-7 | | | | | | | | | | | | | | | |

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to prepare a broadly reactive influenza B virus hemagglutinin (HA), comprising:
introducing random mutations at a plurality of nucleotides in an isolated parental influenza virus nucleic acid molecule encoding an influenza B virus hemagglutinin (HA), thereby providing a library of influenza virus nucleic acid molecules encoding a mutant influenza B virus hemagglutinin, wherein the plurality of mutations in the influenza B virus hemagglutinin results in a plurality of amino acid substitutions or a deletion of at least one amino acid, at an amino acid position including positions 29, 34, 38, 40, 48, 56, 58, 71, 73, 75, 76, 80, 81, 89, 108, 116, 117, 121, 122, 125, 126, 129, 133, 136, 137, 139, 146, 148, 149, 150, 154, 157, 162, 163, 164, 165, 166, 167, 168, 170, 172, 173, 174, 175, 177, 179, 180, 182, 183, 184, 197, 198, 199, 201, 202, 203, 206, 208, 209, 216, 220, 222, 229, 230, 232, 233, 235, 238, 251, 253, 254, 255, 256, 261, 262, 266, 267, 286, 298, 299, 312, 332, 368, 384, 415, 422, 479, 498, 505, 551, 555, or 559, or any combination thereof (Yamagata HA numbering);
introducing the library into cells so as to provide a library of cells that express the mutant hemagglutinins; and
identifying a mutant hemagglutinin encoded by the library that is recognized by anti-Yamagata lineage specific sera and anti-Victoria lineage specific sera, or provides for protection after immunization and challenge with a Yamagata lineage virus and a Victoria lineage virus, as a result of one or more substitutions and/or deletions at the one or more of the positions.

2. The method of claim 1 wherein the parental influenza virus nucleic acid molecule encodes a Yamagata lineage hemagglutinin as defined by phylogenetic analysis of human influenza B viruses or wherein the parental influenza virus nucleic acid molecule encodes a Victoria lineage hemagglutinin as defined by phylogenetic analysis of human influenza B viruses.

3. (canceled)

4. The method of claim 1 wherein the mutant hemagglutinin has at up to 5, 10 or 15 substitutions relative to the parent hemagglutinin, wherein the mutant hemagglutinin has at up to 15, 20, 21, 22, 23, 24 or 25 substitutions relative to the parent hemagglutinin or wherein the mutant hemagglutinin has at least one amino acid deletion relative to the parent hemagglutinin, or any combination thereof.

5-8. (canceled)

9. The method of claim 1 wherein the mutant hemagglutinin has an amino acid at position 40 that is Y or H; position 48 that is R or E; position 56 that is D or K; position 71 that is M or K; position 73 that is V or T; position 75 that is T or K; position 76 that is T or I; position 80 that is K or R; position 81 that is A or V; position 116 that is K or H; position 117 that is I or V; position 122 that is Q or H; position 126 that is D or N; position 129 that is K or D; position 133 that is G or R; position 136 that is R or E; position 137 that is L or I; position 146 that is A or 1; position 148 that is S or N; position 149 that is K or G; position 150 that is I or N; position 162 that is K or a deletion; position 163 that is D or a deletion; position 164 that is N or K; position 165 that is Y or N; position 167 that is N or T; position 174 that is V or I; position 197 that is K or E; position 201 that is K or A; position 202 that is S or K; position 208 that is N or K; position 229 that is D or G; position 232 that is D or N; position 251 that is M or V; position 254 that is P or S; position 261 that is V or T; position 266 that is V or I; position 298 that is E or K; or position 312 that is K or E, or a combination thereof, or wherein the mutant hemagglutinin has an amino acid at position 40 that is H; position 48 that is E; position 56 that is D; position 71 that is K; position 73 that is V; position 75 that is K; position 76 that is T; position 80 that is K or R; position 81 that is V; position 116 that is H; position 117 that is I or V; position 122 that is Q or H; position 126 that is D; position 129 that is K; position 133 that is G; position 136 that is R or E; position 137 that is I; position 146 that is A; position 149 that is K or G; position 148 that is S or N; position 150 that is I; position 162 that is K; position 163 that is D or as deletion; position 164 that is K; position 165 that is N; position 167 that is N; position 174 that is V or I; position 197 that is K or E; position 201 that is K or A; position 202 that is K; position 208 that is K; position 229 that is D; position 232 that is D or N; position 251 that is M or V; position 254 that is P; position 261 that is T; position 266 that is V or I; position 298 that is E; or position 312 that is K or E, or a combination thereof, wherein the mutant hemagglutinin has an amino acid at position 40 that is H; position 48 that is E; position 71 that is K; position 75 that is K; position 76 that is T; position 80 that is K or R; position 81 that is V; position 116 that is H; position 117 that is I or V; position 122 that is Q or H; position 129 that is K; position 133 that is G; position 136 that is R or E; position 137 that is I; position 149 that is K or G; position 148 that is S or N; position 162 that is K; position 163 that is D; position 164 that is K; position 165 that is N; position 167 that is N; position 174 that is V or I; position 201 that is K or A; position 202 that is K; position 208 that is K; position 229 that is D; position 251 that is M or V; position 254 that is P; position 261 that is T; position 298 that is E, or a combination thereof, or wherein the mutant hemagglutinin has an amino acid at position 40 that is H; position 48 that is E; position 71 that is K; position 75 that is K; position 76 that is T; position 80 that is K or R; position 81 that is V; position 116 that is H; position 117 that is I or V; position 122 that is Q or H; position 129 that is K; position 133 that is G; position 136 that is R or E; position 137 that is I; position 149 that is K or G; position 148 that is S or N; position 162 that is K; position 163 that is D; position 164 that is K; position 165 that is N; position 167 that is N; position 174 that is V or I; position 201 that is K or A; position 202 that is K; position 208 that is K; position 229 that is D; position 251 that is M or V; position 254 that is P; position 261 that is T; position 298 that is E, or a combination thereof, or wherein the mutant hemagglutinin has 14 to 24 substitutions selected from position 40 is Y; position 48 is R; position 56 is D; position 71 is M; position 73 is V; position 75 is T; position 76 is T; position 80 is K; position 81 is A; position 116 is K; position 117 is I; position 122 is Q; position 126 is D; position 129 is K; position 133 is G; position 136 is R; position 137 is L; position 146 is A; position 148 is S; position 149 is K; position 150 is I; position 162 is K; position 163 is D; position 164 is N; position 165 is; position 167 is N; position 174 is V; position 197 is K; position 201 is K; position 202 is S; position 208 is N; position 229 is D; position 232 is D; position 251 is M; position 254 is P; position 261 is V; position 266 is V; position 298 is E; or position 312 is K, or a combination thereof, or wherein a majority of the substitutions in the mutant hemagglutinin include when position 40 is H; position 48 is E; position 56 is K; position 71 is K; position 73 is T; position 75 is K; position 76 is I; position 80 is R; position 81 is V; position 116 is H; position 117 is V; position 122 is H; position 126 is N; position 129 is D; position 133 is R; position 136 is E; position 137 is I; position 146 is I; position 148 is N; position 149 is G; position 150 is N; position 162 is a deletion; position 163 is a deletion; position 164 is K; position 165 is N; position 167 is T; position 174 is I; position 197 is E; position 201 is A; position 202 is K; position 208 is K; position 229 is G; position 232 is N; position 251 is V; position 254 is S; position 261 is T; position 266 is 1; position 298 is K; or position 312 is E, or a combination thereof, or wherein the mutant hemagglutinin has 13 to 22 substitutions or one or more deletions selected from position 40 is H; position 48 is E; position 56 is K; position 71 is K; position 73 is T; position 75 is K; position 76 is I; position 80 is R; position 81 is V; position 116 is H; position 117 is V; position 122 is H; position 126 is N; position 129 is D; position 133 is R; position 136 is E; position 137 is I; position 146 is I; position 148 is N; position 149 is G; position 150 is N; position 162 is a deletion; position 163 is a deletion; position 164 is K; position 165 is N; position 167 is T; position 174 is I; position 197 is E; position 201 is A; position 202 is K; position 208 is K; position 229 is G; position 232 is N; position 251 is V; position 254 is S; position 261 is T; position 266 is 1; position 298 is K; or position 312 is E, or a combination thereof, or wherein the position is when position 40 is Y; position 48 is R; position 56 is D; position 71 is M; position 73 is V; position 75 is T; position 76 is T; position 80 is K; position 81 is A; position 116 is K; position 117 is I; position 122 is Q; position 126 is D; position 129 is K; position 133 is G; position 136 is R; position 137 is L; position 146 is A; position 148 is S; position 149 is K; position 150 is I; position 162 is K; position 163 is D; position 164 is N; position 165 is; position 167 is N; position 174 is V; position 197 is K; position 201 is K; position 202 is S; position 208 is N; position 229 is D; position 232 is D; position 251 is M; position 254 is P; position 261 is V; position 266 is V; position 298 is E; or position 312 is K, or any combination thereof.

10. The method of claim 1 wherein the mutant hemagglutinin has an amino acid at position 40 that is H; position 48 that is E; position 56 that is D; position 71 that is K; position 73 that is V; position 75 that is K; position 76 that is T; position 80 that is K or R; position 81 that is V; position 116 that is H; position 117 that is I or V; position 122 that is Q or H; position 126 that is D; position 129 that is K; position 133 that is G; position 136 that is R or E; position 137 that is I; position 146 that is A; position 149 that is K or G; position 148 that is S or N; position 150 that is I; position 162 that is K; position 163 that is D or as deletion; position 164 that is K; position 165 that is N; position 167 that is N; position 174 that is V or I; position 197 that is K or E; position 201 that is K or A; position 202 that is K; position 208 that is K; position 229 that is D; position 232 that is D or N; position 251 that is M or V; position 254 that is P; position 261 that is T; position 266 that is V or I; position 298 that is E; or position 312 that is K or E, or a combination thereof.

11. The method of claim 1 wherein the mutant hemagglutinin has an amino acid at position 40 that is H; position 48 that is E; position 71 that is K; position 75 that is K; position 76 that is T; position 80 that is K or R; position 81 that is V; position 116 that is H; position 117 that is I or V; position 122 that is Q or H; position 129 that is K; position 133 that is G; position 136 that is R or E; position 137 that is I; position 149 that is K or G; position 148 that is S or N; position 162 that is K; position 163 that is D; position 164 that is K; position 165 that is N; position 167 that is N; position 174 that is V or I; position 201 that is K or A; position 202 that is K; position 208 that is K; position 229 that is D; position 251 that is M or V; position 254 that is P; position 261 that is T; position 298 that is E, or a combination thereof.

12-15. (canceled)

16. The method of claim 1 wherein the substitutions are at position 40, 48, 116, 126, 136, 137, 164, 197, 202, 208, 232, 251 or 261, or any combination thereof, or wherein the substitutions are at position 56, 71, 73, 75, 76, 81, 146, 174, 201, 266, 298, 312, or the deletion is at position 162 or 163, or any combination thereof.

17. (canceled)

18. A method to prepare an influenza B virus hemagglutinin that is recognized by anti-Yamagata lineage specific sera and anti-Victoria lineage specific sera, comprising:
isolating or preparing nucleic acid having two or more mutations in a parental influenza B virus HA nucleic acid molecule at two or more codons for an amino acid at position 40, 48, 56, 71, 73, 75, 76, 80, 81, 116, 117, 122, 126, 129, 133, 136, 137, 146, 148, 149, 150, 157, 162, 163, 164, 165, 167, 174, 197, 201, 202, 206, 208, 229, 232, 251, 254, 261, 266, 298, or 312, or any combination thereof, thereby providing a mutated HA nucleic acid molecule.

19. The method of claim 18 wherein the parent hemagglutinin is a Yamagata lineage hemagglutinin or wherein the parent hemagglutinin is a Victoria lineage hemagglutinin.

20. (canceled)

21. The method of claim 18 wherein the mutant hemagglutinin has at up to 5, 10 or 15 substitutions relative to the parent hemagglutinin, wherein the mutant hemagglutinin has at up to 15, 20, 21, 22, 23, 24 or 25 substitutions relative to the parent hemagglutinin or wherein the mutant hemagglutinin has at least one amino acid deletion relative to the parent hemagglutinin.

22-24. (canceled)

25. The method of claim 18 wherein the mutant hemagglutinin has an amino acid at position 40 that is Y or H; position 48 that is R or E; position 56 that is D or K; position 71 that is M or K; position 73 that is V or T; position 75 that is T or K; position 76 that is T or I; position 80 that is K or R; position 81 that is A or V; position 116 that is K or H; position 117 that is I or V; position 122 that is Q or H; position 126 that is D or N; position 129 that is K or D; position 133 that is G or R; position 136 that is R or E; position 137 that is L or I; position 146 that is A or I; position 148 that is S or N; position 149 that is K or G; position 150 that is I or N; position 162 that is K or a deletion; position 163 that is D or a deletion; position 164 that is N or K; position 165 that is Y or N; position 167 that is N or T; position 174 that is V or I; position 197 that is K or E; position 201 that is K or A; position 202 that is S or K; position 208 that is N or K; position 229 that is D or G; position 232 that is D or N; position 251 that is M or V; position 254 that is P or S; position 261 that is V or T; position 266 that is V or I; position 298 that is E or K; or position 312 that is K or E, or a combination thereof, or wherein a majority of the substitutions in the mutant hemagglutinin are position 40 is Y; position 48 is R; position 56 is D; position 71 is M; position 73 is V; position 75 is T; position 76 is T; position 80 is K; position 81 is A; position 116 is K; position 117 is I; position 122 is Q; position 126 is D; position 129 is K; position 133 is G; position 136 is R; position 137 is L; position 146 is A; position 148 is S; position 149 is K; position 150 is I; position 162 is K; position 163 is D; position 164 is N; position 165 is; position 167 is N; position 174 is V; position 197 is K; position 201 is K; position 202 is S; position 208 is N; position 229 is D; position 232 is D; position 251 is M; position 254 is P; position 261 is V; position 266 is V; position 298 is E; or position 312 is K, or a combination thereof, or wherein the mutant hemagglutinin has 16 to 24 substitutions selected from position 40 is Y; position 48 is R; position 56 is D; position 71 is M; position 73 is V; position 75 is T; position 76 is T; position 80 is K; position 81 is A; position 116 is K; position 117 is I; position 122 is Q; position 126 is D; position 129 is K; position 133 is G; position 136 is R; position 137 is L; position 146 is A; position 148 is S; position 149 is K; position 150 is I; position 162 is K; position 163 is D; position 164 is N; position 165 is; position 167 is N; position 174 is V; position 197 is K; position 201 is K; position 202 is S; position 208 is N; position 229 is D; position 232 is D; position 251 is M; position 254 is P; position 261 is V; position 266 is V; position 298 is E; or position 312 is K, or a combination thereof, or wherein a majority of the substitutions in the mutant hemagglutinin are position 40 is H; position 48 is E; position 56 is K; position 71 is K; position 73 is T; position 75 is K; position 76 is I; position 80 is R; position 81 is V; position 116 is H; position 117 is V; position 122 is H; position 126 is N; position 129 is D; position 133 is R; position 136 is E; position 137 is I; position 146 is I; position 148 is N; position 149 is G; position 150 is N; position 162 is a deletion; position 163 is a deletion; position 164 is K; position 165 is N; position 167 is T; position 174 is I; position 197 is E; position 201 is A; position 202 is K; position 208 is K; position 229 is G; position 232 is N; position 251 is V; position 254 is S; position 261 is T; position 266 is I; position 298 is K; or position 312 is E, or a combination thereof, or wherein the mutant hemagglutinin has 13 to 22 substitutions or deletions selected from of position 40 is H; position 48 is E; position 56 is K; position 71 is K; position 73 is T; position 75 is K; position 76 is I; position 80 is R; position 81 is V; position 116 is H; position 117 is V; position 122 is H; position 126 is N; position 129 is D; position 133 is R; position 136 is E; position 137 is I; position 146 is I; position 148 is N; position 149 is G; position 150 is N; position 162 is a deletion; position 163 is a deletion; position 164 is K; position 165 is N; position 167 is T; position 174 is I; position 197 is E; position 201 is A; position 202 is K; position 208 is K; position 229 is G; position 232 is N; position 251 is V; position 254 is S; position 261 is T; position 266 is I; position 298 is K; or position 312 is E, or a combination thereof, or wherein the mutant hemagglutinin has an amino acid at position 40 that is H; position 48 that is E; position 56 that is D; position 71 that is K; position 73 that is V; position 75 that is K; position 76 that is T; position 80 that is K or R; position 81 that is V; position 116 that is H; position 117 that is I or V; position 122 that is Q or H; position 126 that is D; position 129 that is K; position 133 that is G; position 136 that is R or E; position 137 that is I; position 146 that is A; position 149 that is K or G; position 148 that is S or N; position 150 that is I; position 162 that is K; position 163 that is D or as deletion; position 164 that is K; position 165 that is N; position 167 that is N; position 174 that is V or I; position 197 that is K or E; position 201 that is K or A; position 202 that is K; position 208 that is K; position 229 that is D; position 232 that is D or N; position 251 that is M or V; position 254 that is P; position 261 that is T; position 266 that is V or I; position 298 that is E; or position 312 that is K or E, or a combination thereof, or wherein the mutant hemagglutinin has an amino acid at position 40 that is H; position 48 that is E; position 71 that is K; position 75 that is K; position 76 that is T; position 80 that is K or R; position 81 that is V; position 116 that is H; position 117 that is I or V; position 122 that is Q or H; position 129 that is K; position 133 that is G; position 136 that is R or E; position 137 that is I; position 149 that is K or G; position 148 that is S or N; position 162 that is K; position 163 that is D; position 164 that is K; position 165 that is N; position 167 that is N; position 174 that is V or I; position 201 that is K or A; position 202 that is K; position 208 that is K; position 229 that is D; position 251 that is M or V; position 254 that is P; position 261 that is T; position 298 that is E; or a combination thereof.

26-31. (canceled)

32. The method of claim 18 wherein the substitutions are at position 40, 48, 116, 126, 136, 137, 164, 197, 202, 208, 232, 251 or 261, or any combination thereof, or wherein the substitutions are at position 56, 71, 73, 75, 76, 81, 146, 174, 201, 266, 298, 312, or the deletion is at position 162 or 163, or any combination thereof.

33. (canceled)

34. A composition comprising a recombinant influenza B virus encoding a hemagglutinin comprising a plurality of mutations relative to a parent virus hemagglutinin, wherein the recombinant influenza B virus HA comprises one or more substitutions at positions 40, 48, 56, 71, 73, 75, 76, 80, 81, 116, 117, 122, 126, 129, 133, 136, 137, 146, 148, 149, 150, 157, 162, 165, 167, 174, 197, 201, 202, 206, 208, 229, 232, 251, 254, 261, 266, 298, or 312, or comprises one or more deletions at position 163 or 164, or any combination thereof, relative to a Yamagata lineage hemagglutinin or a Victoria lineage hemagglutinin.

35-36. (canceled)

37. The composition of claim 34 wherein the mutant hemagglutinin has at up to 5, 10 or 15 substitutions relative to the parent hemagglutinin, wherein the mutant hemagglutinin has at up to 15, 20, 21, 22, 23, 24 or 25 substitutions relative to the parent hemagglutinin or wherein the mutant hemagglutinin has at least one amino acid deletion relative to the parent hemagglutinin.

38-40. (canceled)

41. The composition of claim 34 wherein the mutant hemagglutinin has an amino acid at position 40 that is Y or H; position 48 that is R or E; position 56 that is D or K; position 71 that is M or K; position 73 that is V or T; position 75 that is T or K; position 76 that is T or I; position 80 that is K or R; position 81 that is A or V; position 116 that is K or H; position 117 that is I or V; position 122 that is Q or H; position 126 that is D or N; position 129 that is K or D; position 133 that is G or R; position 136 that is R or E; position 137 that is L or I; position 146 that is A or I; position 148 that is S or N; position 149 that is K or G; position 150 that is I or N; position 162 that is K or a deletion; position 163 that is D or a deletion; position 164 that is N or K; position 165 that is Y or N; position 167 that is N or T; position 174 that is V or 1; position 197 that is K or E; position 201 that is K or A; position 202 that is S or K; position 208 that is N or K; position 229 that is D or G; position 232 that is D or N; position 251 that is M or V; position 254 that is P or S; position 261 that is V or T; position 266 that is V or 1; position 298 that is E or K; or position 312 that is K or E, or a combination thereof, or wherein a majority of the substitutions in the mutant hemagglutinin are position 40 is Y; position 48 is R; position 56 is D; position 71 is M; position 73 is V; position 75 is T; position 76 is T; position 80 is K; position 81 is A; position 116 is K; position 117 is I; position 122 is Q; position 126 is D; position 129 is K; position 133 is G; position 136 is R; position 137 is L; position 146 is A; position 148 is S; position 149 is K; position 150 is I; position 162 is K; position 163 is D; position 164 is N; position 165 is; position 167 is N; position 174 is V; position 197 is K; position 201 is K; position 202 is S; position 208 is N; position 229 is D; position 232 is D; position 251 is M; position 254 is P; position 261 is V; position 266 is V; position 298 is E; or position 312 is K, or a combination thereof, or wherein the mutant hemagglutinin has 16 to 24 substitutions selected from position 40 is Y; position 48 is R; position 56 is D; position 71 is M; position 73 is V; position 75 is T; position 76 is T; position 80 is K; position 81 is A; position 116 is K; position 117 is I; position 122 is Q; position 126 is D; position 129 is K; position 133 is G; position 136 is R; position 137 is L; position 146 is A; position 148 is S; position 149 is K; position 150 is I; position 162 is K; position 163 is D; position 164 is N; position 165 is; position 167 is N; position 174 is V; position 197 is K; position 201 is K; position 202 is S; position 208 is N; position 229 is D; position 232 is D; position 251 is M; position 254 is P; position 261 is V; position 266 is V; position 298 is E; or position 312 is K, or a combination thereof, or wherein a majority of the substitutions in the mutant hemagglutinin are position 40 is H; position 48 is E; position 56 is K; position 71 is K; position 73 is T; position 75 is K; position 76 is I; position 80 is R; position 81 is V; position 116 is H; position 117 is V; position 122 is H; position 126 is N; position 129 is D; position 133 is R; position 136 is E; position 137 is I; position 146 is I; position 148 is N; position 149 is G; position 150 is N; position 162 is a deletion; position 163 is a deletion; position 164 is K; position 165 is N; position 167 is T; position 174 is I; position 197 is E; position 201 is A; position 202 is K; position 208 is K; position 229 is G; position 232 is N; position 251 is V; position 254 is S; position 261 is T; position 266 is I; position 298 is K; or position 312 is E, or a combination thereof, or wherein the mutant hemagglutinin has 13 to 22 substitutions or deletions selected from of position 40 is H; position 48 is E; position 56 is K; position 71 is K; position 73 is T; position 75 is K; position 76 is I; position 80 is R; position 81 is V; position 116 is H; position 117 is V; position 122 is H; position 126 is N; position 129 is D; position 133 is R; position 136 is E; position 137 is I; position 146 is I; position 148 is N; position 149 is G; position 150 is N; position 162 is a deletion; position 163 is a deletion; position 164 is K; position 165 is N; position 167 is T; position 174 is I; position 197 is E; position 201 is A; position 202 is K; position 208 is K; position 229 is G; position 232 is N; position 251 is V; position 254 is S; position 261 is T; position 266 is I; position 298 is K; or position 312 is E, or a combination thereof.

46-47. (canceled)

48. The composition of claim 34 which further comprises one or more other influenza viruses or one or more antigens thereof.

49. The composition of claim 48 wherein the one or more other influenza viruses comprise one or more influenza A viruses.

50. The composition of claim 34 wherein at least one of the positions include when position 40 is H; position 48 is E; position 56 is D or K; position 71 is K; position 73 is T or V; or position 75 is K, or any combination thereof, or, wherein at least two of the position include when position 76 is I or T; 80 is R; 81 is K or V; 116 is H; 117 is V or I; 122 is H or Q; or 126 is D or N, or wherein at least four of the positions include when position 129 is K or D; 133 is G or R; 136 is R or E; 137 is 1; 146 is I or A; 148 is N or S; 149 is G or K; 150 is N or I; 162 is a deletion or K; or 163 is deletion or D, or wherein at least five of the positions include when position 164 is K; 165 is N; 167 is N; 174 is I; 197 is E or K; 201 is A or K; 202 is K; 208 is K; 229 is D; 232 is D or N; 251 is V or M; 254 is P; 261 is T; 266 is I or I; 298 is E; or 312 is K, or wherein the mutant hemagglutinin has an amino acid at position 40 that is H; position 48 that is E; position 56 that is D; position 71 that is K; position 73 that is V; position 75 that is K; position 76 that is T; position 80 that is K or R; position 81 that is V; position 116 that is H; position 117 that is I or V; position 122 that is Q or H; position 126 that is D; position 129 that is K; position 133 that is G; position 136 that is R or E; position 137 that is I; position 146 that is A; position 149 that is K or G; position 148 that is S or N; position 150 that is I; position 162 that is K; position 163 that is D or as deletion; position 164 that is K; position 165 that is N; position 167 that is N; position 174 that is V or I; position 197 that is K or E; position 201 that is K or A; position 202 that is K; position 208 that is K; position 229 that is D; position 232 that is D or N; position 251 that is M or V; position 254 that is P; position 261 that is T; position 266 that is V or I; position 298 that is E; or position 312 that is K or E, or a combination thereof, or wherein the mutant hemagglutinin has an amino acid at position 40 that is H; position 48 that is E; position 71 that is K; position 75 that is K; position 76 that is T; position 80 that is K or R; position 81 that is V; position 16 that is H; position 117 that is I or V; position 122 that is Q or H; position 129 that is K; position 133 that is G; position 136 that is R or E; position 137 that is I; position 149 that is K or G; position 148 that is S or N; position 162 that is K; position 163 that is D; position 164 that is K; position 165 that is N; position 167 that is N; position 174 that is V or I; position 201 that is K or A; position 202 that is K; position 208 that is K; position 229 that is D; position 251 that is M or V; position 254 that is P; position 261 that is T; position 298 that is E, or a combination thereof.

51-55. (canceled)

56. The composition of claim 34 wherein the virus does not bind to sera specific for one of SEQ ID Nos. 1-3.

57-62. (canceled)

63. A method to immunize an animal, comprising: administering an effective amount of the composition of claim 34.

64-75. (canceled)


SEQUENCE LISTING (22 sequences)

SEQ ID NO: 1 - DNA, 1755 bp, Influenza b virus
  source 1..1755: mol_type = other DNA
atgaaggcaataattgtactactcatggtagtaacatccaatgcagatcgaatctgcactgggataacatcgtcaaactcaccccatgtggtcaaaactgctactcaaggggaagtcaatgtgactggtgtgataccactgacaacaacacctaccaaatctcattttgcaaatctcaaaggaacacagaccagaggaaaactatgcccaaactgtctcaactgcacagatctggacgtggccttgggcagaccaaagtgtatggggaccataccttcggcaaaagtttcaatactccacgaagtcaaacctgttacatctgggtgctttcctataatgcacgacagaacaaaaatcagacaactacctaatcttctcagaggatatgaaaatatcaggttatcaacccgtaacgttatcaatgcagaaacggcaccaggaggaccctacacagttggaacctcaggatcttgccctaacgttaccaatgggaaaggattcttcgaaacaatggcttgggctgtcccaaaaaacaaaaacaaaacagcaacgaatccattaacagtagaagtaccatacatttgtacaaaaggagaagaccaaattactgtttgggggttccactctgatgacgaaacccaaatggtaatactctatggagactcgaagcctcaaaagttcacctcatctgccaatggagtaaccacacattatgtttctcagattggtggcttcccaaatcaaacagaagacgaagggctaaaacaaagcggcagaattgttgttgattacatagtgcaaaaacctggaaaaacaggaacaattgtctatcaaagaggtgttttattgcctcaaaaagtgtggtgcgcaagtggcaggagcaaggtaataaaagggtccttgcctttaattggtgaagcagattgcctccacgaaaaatacggtggattaaacaaaagcaagccttactacacaggagaacatgcaaaagccataggaaattgcccaatatgggtgaaaacacccttgaaactggccaatggaaccaaatatagaccgcctgcaaaactattaaaggaaaggggtttcttcggagctattgctggtttcttggaaggaggatgggaaggaatgattgcaggttggcacggatacacatctcatggagcacatggagtggcagtggcagcggaccttaagagtacgcaagaagctataaacaagataacaaaaaatctcaattctttaagtgagctagaagtaaaaaatcttcaaagactaagcggagcaatggatgaactccacaacgaaatactcgagctggatgagaaggtggatgatctaagagctgatacaataagctcacaaatagagcttgcagtcttgctttccaacgaaggaataataaacagtgaagatgagcatctcttggcacttgaaagaaaactgaagaaaatgctgggcccctctgctgtagacatagggaatggatgcttcgaaaccaaacacaaatgcaaccagacttgcctagacaggatagctgctggcacctttaatgcaggagaattttctcttcccacttttgattcattaaatattactgctgcatctttaaatgatgatggattggataatcatactatactgctctactactcaactgctgcttctagcttggctgtaacattgatgatagctatcttcattgtttacatggtctccagagacaatgtttcttgttccatctgtctataa

SEQ ID NO: 2 - DNA, 1749 bp, Influenza b virus
  source 1..1749: mol_type = other DNA
atgaaggcaataattgtactactcatggtagtaacatccaatgcagatcgaatctgcactgggataacatcgtcaaactcaccccatgtggtcaaaactgccactcaaggagaagtcaatgtgactggtgtgataccactgacaacaacacctaccagatctcattttgcaaatctcaaaggaacacagaccagaggaaaactatgcccaaaatgtttcaactgcacagatctggacgtggccttgggcagaccaaaatgcatggggaacataccttctgcaaaagtttcaatactccatgaagtcaaacctgttacatctggatgctttcctataatgcacgacagaacaaaaatcagacaactatctaatcttctcagaggatatgaaaatatcaggttatcaaccagcaacgttatcaatgcagagacggcaccaggaggaccctacaaagttggaacctcagggtcttgccctaacgttactaatgggaacggcttcttcgaaacaatggcttgggctgttccaaaaaacaaaacagcaatgaatccagtaacagtagaagtaccatacatttgtgcagaaggggaagaccaaattactgtttgggggttccactctgatgacgaaacccaaatgggaagactctatggagactcaaagcctcaaaagttcacctcatatgccaatggagtaaccacacattatgtttctcagattggtggcttcccagatcaaacagaagacgaggggctgaaacaaagcggcagaattgttgttgattacatagtgcaaaaacctgaaaaaacaggaacaattgtttaccaaagaggtattttattgcctcaaaaagtgtggtgcgcaagtggcagaagcaaggtaataaaagggtccttgcctttaattggtgaagcagattgcctccacgaaaaatacggtggattaaataaaagcaagccttactacacaggagagcatgcaaaggccataggaaattgcccaatatgggtgaaaacacccttgaagctagccaatggaaccaaatatagaccgcctgcaaaactattaaaggaaagaggtttctttggagctattgctggtttcttggaaggaggctgggaaggaatgattgcaggttggcacggatacacatctcatggagcacatggagtggcagtggcagcagaccttaagagtacacaagaagctataaacaagataacaaaaaacctcaactctttgagtgagctagaagtaaaaaatcttcaaaggctaagcggagcaatggatgagcttcacaacgaaatactcgagctagacgagaaggtggatgatctaagagctgatacaataagctcacaaatagagcttgcagtcttgctttccaacgaagggataataaacagtgaagatgaacatctcttggcacttgaaagaaaactgaagaaaatgctgggcccctctgctgtagacatagggaatggatgcttcgaaaccaaacacaaatgcaaccagacttgcctagacaggatagctgctggcacctttagtgcaggagaattttctcttcccacttttgattcattaaacattactgctgcatctttaaatgatgatggattggataatcatactatactgctctactactcaactgctgcttctagcttggctgtaacattgatgatagctatcttcattgtctacatggtctccagagacaatgtttcttgttctatctgtctgtga

SEQ ID NO: 3 - DNA, 1758 bp, Influenza b virus
  source 1..1758: mol_type = other DNA
atgaaggcaataattgtactactcatggtagtaacatccaatgcagatcgaatctgcactgggataacatcgtcaaactcaccccatgtggtcaaaactgctactcaaggggaagttaatgtgactggtgtgataccactgacaacaacacctaccaaatctcattttgcaaatctcaaaggaacacagaccagagggaaactatgcccaaactgtctcaactgcacagatctggacgtggccttgggcagaccaaagtgtatggggaccataccttcggcaaaagcttcaatactccacgaagtcaaacctgttacatctgggtgctttcctataatgcacgacagaacaaaaatcagacagctacccaatcttctcagaggatatgaaaatatcaggttatcagcccgtaacgttatcaacgcagaaacggcaccaggaggaccctacatagttggaacctcaggatcttgccctaacgttaccaatgggaaaggattcttcgcaacaatggcttgggctgtcccaaaaaacaacaaaaacaaaacagcaacgaatccattaacagtagaagtaccatacatttgtacaaaaggagaagaccaaattactgtttgggggttccattctgataacgaaacccaaatggtaacactctatggagactcgaagcctcaaaagttcacctcatctgccaacggagtaaccacacattatgtttctcagattggtggcttcccaaatcaaacagaagacgaagggctaccacaaagcggcagaattgttgttgattacatggtgcaaaaacctggaaaaacaggaacaattgtctatcaaagaggtgttttattgcctcaaaaagtgtggtgcgcaagtggcaggagcaaggtaataaaagggtccttgcctttaattggtgaagcagattgcctccacgaaaaatacggtggattaaacaaaagcaagccttactacacaggagaacatgcaaaagccataggaaattgcccaatatgggtgaaaacacccttgaagctggccaatggaaccaaatatagacctcctgcaaaactattaaaggaaaggggtttcttcggagctattgctggtttcttggaaggaggatgggaaggaatgattgcaggttggcacggatacacatctcatggagcacatggagtggcagtggcagcagaccttaagagtacgcaagaagctataaacaagataacaaaaaatctcaattctttaagtgagctagaagtaaagaatcttcaaagactaagcggtgcaatggatgaactccacaacgaaatactcgagctggatgagaaagtggatgatctcagagctgatacaataagctcgcaaatagagcttgcagtcttgctttccaacgaaggaataataaacagtgaagatgagcatctcttggcacttgaaagaaaactgaagaaaatgctgggcccctctgctgtagacatagggaatggatgcttcgaaaccaaacacaaatgcaaccagacttgcctagacaggatagctgctggcacctttaatgcaggagaattttctcttcccacttttgattcactaaatattactgctgcatctttaaatgatgatggattggataatcatactatactgctctactactcaactgctgcttctagtttggctgtaacattgatgatagctatctttattgtttatatggtctccagagacaatgtttcttgctccatctgtctataa

SEQ ID NO: 4 - DNA, 1755 bp, Influenza b virus
  source 1..1755: mol_type = other DNA
atgaaggcaataattgtactactcatggtagtaacatccaacgcagatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcaggcaactacccaatcttctcagaggatatgaaaagatcaggttatcaacccaaaacgttatcgatgcagaaaaagcaccaggaggaccctacagacttggaacctcaggatcttgccctaacgctaccagtaaaatcggattttttgcaacaatggcttgggctgtcccaaaggacaactacaaaaatgcaacgaacccactaacagtggaagtaccatacatttgtacagaaggggaagaccaaattactgtttgggggttccattcggataacaaaacccaaatgaagagcctctatggagactcaaatcctcaaaagttcacctcatctgctaatggagtaaccacgcattatgtttctcagattggcgacttcccagatcaaacagaagacggaggactaccacaaagcggcagaattgttgttgattacatgatgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctgcaaaactattgaaggaaaggggtttcttcggagctattgctggtttcctagaaggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgatctcagagctgacactataagctcacaaatagaacttgcagtcttgctttccaacgaaggaataataaacagtgaagacgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgattcattgaacattactgctgcatctttaaatgatgatggattggataaccatactatactgctctattactcaactgctgcttctagtttggctgtaacattaatgctagctatttttattgtttatatggtctccagagacaacgtttcatgctccatctgtctataa

SEQ ID NO: 5 - DNA, 1758 bp, Influenza b virus
  source 1..1758: mol_type = other DNA
atgaaggcaataattgtactactcatggtagtaacatccaatgcagatcgaatctgcactgggataacatcgtcaaactcaccacatgtcgtcaaaactgctactcaaggggaggtcaatgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaggaccctacaaaattggaacctcagggtcttgccctaacattaccaatggaaacggattcttcgcaacaatggcttgggccgtcccaaaaaacgacaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggtagaattgttgttgattacatggtgcaaaaatctggaaaaacaggaacaattacctatcaaagaggtattttattgcctcaaaaggtgtggtgcgcaagtggcaggagcaaggtaataaaaggatccttgcctttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagacctcctgcaaaactattaaaggaaaggggtttcttcggagctattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcacttaatattactgctgcatctttaaatgacgatggattggataatcatactatactgctttactactcaactgctgcctccagtttggctgtaacactgatgatagctatctttgttgtttatatggtctccagagacaatgtttcttgctccatttgtctataa

SEQ ID NO: 6 - DNA, 1749 bp, Influenza b virus
  source 1..1749: mol_type = other DNA
atgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacaacgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaggtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataa

SEQ ID NO: 7 - protein, 569 aa, Influenza b virus
DRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 8 - protein, 567 aa, Influenza b virus
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 9 - protein, 320 aa, Influenza b virus
DRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNC

SEQ ID NO: 10 - protein, 318 aa, Influenza b virus
DRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNC

SEQ ID NO: 11 - protein, 50 aa, Influenza b virus
DRICTGITSSNSPHVVKTATQGEVNVYGVIPLTTTPTKSXFANLKGTXTR

SEQ ID NO: 12 - protein, 50 aa, Influenza b virus
GKLCPXCLNCTDLDVALGRPXCXGXXPSAXXSILHEVXPVTSGCFPIMHD

SEQ ID NO: 13 - protein, 50 aa, Influenza b virus
RTKIRQLXNLLRGYEXXRLSXXNVIXAEXAPGXPYXXGTSGSCPNXTXXX

SEQ ID NO: 14 - protein, 50 aa, Influenza b virus
GFFXTMAWAVPXXXXXKXATNXXTXEVPXXCXXXEDQITVWGFHSDXXXQ

SEQ ID NO: 15 - protein, 50 aa, Influenza b virus
KXXLYGDSXPQKFTSSANGXTTHYVSQIGXFPXQXEDXGLXQSGRIVVDY

SEQ ID NO: 16 - protein, 50 aa, Influenza b virus
XXQKXXKTGTIXYQRGXLLPQKVWCASGRSKVIKGSLPLIGEADCLHEXY

SEQ ID NO: 17 - protein, 50 aa, Influenza b virus
GGLNKSKPYYTGXHAKAIGNCPIWVKTPLKLANGTKYRPPAKLLKERGFF

SEQ ID NO: 18 - protein, 50 aa, Influenza b virus
GAIAGFLEGGWEGMIAGXHGYTSHGAHGVAVAADLKSTQEAINKITKNLN

SEQ ID NO: 19 - protein, 50 aa, Influenza b virus
SLSELEVKNLQRLSXAMDELHXEILELDEKVDDLRADTISSQIELAVLLS

SEQ ID NO: 20 - protein, 50 aa, Influenza b virus
NEGIINSEDEHLLALERKLKKMLGPSAVXIGNGCFETKHKCNQTCLDXIA

SEQ ID NO: 21 - protein, 50 aa, Influenza b virus
AGTFXAGEFSLPTFDSLNITAASLNDDGLDNHTILLYYSTAASSLAVTLM

SEQ ID NO: 22 - protein, 20 aa, Influenza b virus
XAIFXVYMXSRDNVSCSICL